(19) United States
(12) Patent Application Publication
(10) Pub. No.: US 2022/0392579 A1
(43) Pub. Date: Dec. 8, 2022

(54) CLASSIFIER MODELS TO PREDICT TISSUE OF ORIGIN FROM TARGETED TUMOR DNA SEQUENCING

(71) Applicant: MEMORIAL SLOAN KETTERING CANCER CENTER, New York, NY (US)

(72) Inventors: Michael F. Berger, New York, NY (US); Barry S. Taylor, New York, NY (US); Alexander Penson, New York, NY (US); Niedzica Camacho, New York, NY (US)

(21) Appl. No.: 17/776,498

(22) PCT Filed: Nov. 11, 2020
(86) PCT No.: PCT/US20/59977

§ 371 Date: May 12, 2022

Related U.S. Application Data
(60) Provisional application No. 62/934,848, filed on Nov. 13, 2019.
(60) Provisional application No. 63/104,323, filed on Oct. 22, 2020.

(51) Int. Cl. G16B 40/00; G16B 20/00

(52) U.S. Cl. CPC G16B 40/00; G16B 20/00; C12Q 2600/112; C12Q 2600/156

(57) ABSTRACT

Disclosed are systems and methods for using genomic features revealed by clinical targeted tumor sequencing to predict of tissue of origin. Using machine learning techniques, an algorithmic classifier is constructed and trained on a large cohort of prospectively sequenced tumors to predict cancer type and origin from DNA sequence data obtained at the point of care. Genome-directed reassessment of classifications may prompt tumor type reclassification resulting in altered cancer therapy. The clinical implementation of artificial intelligence to guide tumor type classifications at the point of care can complement standard histopathology and imaging to enable improved classification accuracy.

### CROSS-REFERENCE TO RELATED APPLICATIONS

This application is a U.S. National Stage Application under 35 U.S.C. § 371 of International Patent Application No. PCT/US2020/059977, filed on Nov. 11, 2020, which claims the benefit of and priority to U.S. Provisional Patent Application No. 62/934,848, filed Nov. 13, 2019, and U.S. Provisional Patent Application No. 63/104,323, filed Oct. 22, 2020, the contents of which are incorporated herein by reference in their entireties.

### STATEMENT OF GOVERNMENT SUPPORT

The invention was made with government support under P30-CA008748 and R01 CA204749, awarded by the National Cancer Institute. The government has certain rights to the invention.

### BACKGROUND

Identifying the site of origin for cancer is a central pillar of disease classification that has successfully directed clinical care for more than a century. Even in an era of precision oncology, in which treatment is increasingly informed by the presence or absence of mutant genes responsible for cancer growth and progression, tumor origin remains a critical determinant of tumor biology and therapeutic sensitivity.

### SUMMARY

The present disclosure examines the extent to which genomic features revealed by clinical targeted tumor sequencing permit accurate prediction of tissue of origin. Using machine learning techniques, an algorithmic classifier was constructed and trained on a large cohort of prospectively sequenced tumors to predict cancer type and origin from DNA sequence data obtained at the point of care. In some cases, genome-directed re-assessment of tumor type identification prompted tumor type reclassification resulting in altered therapy for cancer patients. The clinical implementation of artificial intelligence to guide tumor type classification at the point of care can complement standard histopathology and imaging to enable improved predictive accuracy.

Data derived from routine clinical DNA sequencing of tumors may complement approaches to enable improved predictive accuracy. Provided herein is a novel machine learning approach to predict tumor type from DNA sequence data obtained at the point of care, incorporating both discrete molecular alterations and inferred features such as mutational signatures. This algorithm may be trained on tumors representing 22 cancer types selected from a prospectively sequenced cohort of advanced cancer patients.

The correct tumor type was predicted for 74% of patients in the training set as well as an independent cohort of 10,000+ patients. Predictions were assigned probabilities that reflected empirical accuracy, with 43% of cases representing high-confidence predictions (>95% probability). Informative molecular features and feature categories varied widely by tumor type. Genomic analysis of both tumor tissue and plasma cell-free DNA enabled accurate predictions, demonstrating that this approach may be applied in diverse clinical settings including as an adjunct to cancer screening. Applying the method prospectively to patients under active care enabled genome-directed reassessment of tumor classification in challenging clinical scenarios and the selection of more appropriate treatments, which elicited clinical responses. These results indicate that the application of artificial intelligence to predict tissue of origin in oncology can act as a powerful companion to histologic review to provide integrated pathologic classifications, often with critical therapeutic implications.

Provided herein are systems and methods of predicting tissue of origin from targeted tumor DNA sequencing. A computing device may include a classifier model (e.g., a random forest classifier). The computing device may feed the classifier model with a training dataset to train the classifier model. The training dataset may include DNA tumor sequences obtained from a plurality of cancer subjects. Each sequence may include a feature and a category associated with the feature. The feature may correspond to a set of genes. The category may define a nature of alterations to the set of genes. The nature of alterations may include, for example: gene amplification (AMP), chromosome gain, homozygous deletion, hotspot, hotspot allele, chromosome loss, promoter, signature, structural variant (SV), truncation, and variant of unknown significance (VUS), among others.

In one aspect, various embodiments relate to a method for classifying tumor origin sites. The method may comprise sequencing genetic material in a tissue sample from a subject. The method may comprise generating a subject sample dataset comprising one or more subject genes and one or more subject gene alteration categories. The method may comprise applying a predictive model to the subject sample dataset to generate one or more cancer origin site classifications. The predictive model may be trained using a training dataset. The training dataset may be generated from sequence reads corresponding to genetic material from a cohort of study subjects with known cancers. The training dataset may comprise one or more genes, one or more gene alteration categories corresponding to the one or more genes, and/or one or more labels characterizing tumor origin sites for the known cancers of the study subjects in the cohort. The method may comprise storing an association between the subject and the one or more cancer origin site classifications. The association may be stored in one or more data structures.

In various embodiments, the predictive model may be a random forest classification model. A feature set for the predictive model may comprise one or more categories selected from a group consisting of mutations, indels, focal amplifications and deletions, broad copy number gains and losses, structural rearrangements, mutation signatures, mutation rate, and sex. Classifier scores for the predictive model may be calibrated using multinomial logistic regression to match empirically observed classification probabilities.

In various embodiments, the method may comprise training the predictive model. The predictive model or components thereof may be trained using supervised learning, unsupervised learning, and/or semi-supervised learning. The method may comprise generating the training dataset. Generating the training dataset may comprise acquiring, from a sequencing device, the sequence reads corresponding to the genetic material from the study subjects in the cohort, and using the sequence reads to generate the training dataset. The cohort may exclude certain study subjects, such as study subjects with rare cancers (e.g., cancers not among the top 30 most common cancer types). The training dataset may comprise gene alteration categories comprising one or more selected from a group consisting of gene amplification (AMP), chromosome gain, homozygous deletion, hotspot, allele, chromosome loss, promoter, signature, structural variant (SV), truncation, and variant of unknown significance (VUS). The one or more labels may indicate whether a set of genes in the training dataset is from a cancer subject in the cohort of study subjects.

In various embodiments, the predictive model may be configured to accept data on genes and gene alterations as inputs and to provide one or more cancer origin site classifications as output. The one or more cancer origin site classifications may identify at least one of an internal organ of the subject and/or a cancer type. The predictive model may be configured to generate a confidence score for each cancer origin site classification. Each confidence score may correspond with a likelihood of a cancer origin site for a tumor.

In another aspect, various embodiments relate to a system for classifying tumor origin sites. The system may comprise a computing device having one or more processors. The processors may be configured to acquire sequence reads corresponding to genetic material in a tissue sample from a subject. The sequence reads may be acquired from or via a sequencing device. The processors may be configured to generate a subject sample dataset comprising one or more subject genes and one or more subject gene alteration categories. The subject sample dataset may be generated using the sequence reads. The processors may be configured to apply a predictive model to the subject sample dataset to generate one or more cancer origin site classifications. The predictive model may be trained using a training dataset generated using sequence reads corresponding to genetic material from a cohort of study subjects with known cancers. The training dataset may comprise one or more genes, one or more gene alteration categories corresponding to the one or more genes, and/or one or more labels characterizing tumor origin sites for the known cancers of the study subjects in the cohort. The processors may be configured to store an association between the subject and the one or more cancer origin site classifications. The association may be stored in one or more data structures.

In various embodiments, the predictive model may be a random forest classification model. The processors may be configured to train the predictive model. The processors may be configured to train the predictive model by acquiring the sequence reads corresponding to the genetic material from the study subjects in the cohort. The processors may be configured to acquire the sequence reads from the sequencing device. The processors may be configured to generate the training dataset using the sequence reads corresponding to the genetic material from the study subjects in the cohort. The predictive model may be trained such that it is configured to accept data on genes and gene alterations as inputs and to provide one or more cancer origin site classifications as output. The predictive model may be configured to generate a confidence score for each cancer origin site classification. Each confidence score may correspond with a likelihood of a cancer origin site for a tumor.

In another aspect, various embodiments may relate to a system for determining sites of origin for cancer based on sequencing of genes. The system may comprise one or more processors. The processors may be configured to obtain a training dataset comprising a plurality of sample-derived genetic sequences corresponding to a plurality of cancer subjects. Each sample may define a set of genes and a category. The category of each sample may define at least one alteration to the set of genes and/or at least one genomic alteration in the sample. The processors may be configured to train a classification model configured to generate likelihoods for corresponding cancer origin sites. The classification model may be trained using the plurality of sample genetic sequences. The processors may be configured to acquire a genetic sequence corresponding to a subject. The genetic sequence may be acquired via a sequencer. The genetic sequence may include a set of genes and a category. The category of the genetic sequence may define a nature of alteration to the set of genes in the genetic sequence. The processors may be configured to apply the classification model to the genetic sequence to determine a set of likelihoods for a corresponding set of origin sites of cancers. Each likelihood may indicate a probability measure that the genetic sequence correlates with a presence of cancer at a corresponding origin site.

In various embodiments, the classification model may be trained as a random forest classification model. The processors may be configured to generate the training dataset using sequence reads from the sequencer.

### BRIEF DESCRIPTION OF THE DRAWINGS

The objects, aspects, features, and advantages of the disclosure will become more apparent and better understood by referring to the following description taken in conjunction with the accompanying drawing, in which:

FIGS. 1A-1E. Classifier performance across cancers. FIG. 1A-C: Schematic of random forest classifier. Molecular alterations from MSK-IMPACT sequencing of patients identified or known to have one of 22 tumor types were used to train the classifier. For a given combination of genomic features, the classifier returns a calibrated probability of each tumor type. FIG. 1D: Performance of the classifier across 22 cancer types. True (established) cancer types are displayed horizontally, and predicted cancer types are displayed vertically. The number of tumors for each cancer type in the cohort is shown at the top, and sensitivity and specificity of predictions are indicated at top and right. FIG. 1E: The fraction of samples (vertical axis) with the correct prediction made at or above a given probability (horizontal axis) within each cancer type. Dark hatched bars indicate the fraction of tumors correctly predicted with very high confidence at >95% probability; light hatched bars indicate the additional fraction predicted at >50% probability.

FIG. 2A depicts a block diagram of a system to determine sites of origin for cancer based on sequencing of genes in accordance with an illustrative embodiment.

FIG. 2B depicts example approaches for training and applying predictive models for determining sites of origin in accordance with illustrative embodiments.

FIGS. 3A-3D. Predictive power of molecular features and feature classes. FIG. 3A: Relative information content of different feature categories as shown by the Cohen's kappa metric as a measure of overall accuracy. Diamonds represent the accuracy of a classifier built for each individual feature category as indicated; Circles represent the accuracy upon incrementally adding feature categories (top to bottom). ‘Mutations’ encompass hotspots and non-hotspots. ‘CNA’=copy number alterations. FIG. 3B: Relative importance of different feature categories in different cancer types. Circle size represents the mean contribution of the features in each category to accurate predictions in each cancer type. FIG. 3C: Selected individual features for predicting breast cancer and non-small cell lung cancer in the study cohort, and their relative contribution. Informative features driving correct predictions in all tumor types are shown in FIGS. 1A-1C. ‘VUS’=variants of unknown significance. FIG. 3D: Different features contributing to tumor type predictions in BRAF V600E-mutant colorectal cancer, melanoma, and thyroid cancer, establishing the value of feature interactions to inform tumor type prediction in a cohort of patients that nevertheless share a common molecular alteration.

FIGS. 4A-4E. Most informative features for each tumor type. The 10 most informative individual features for predicting each of the 22 tumor types are shown. Different mutation classes, broad and focal copy number alterations, structural variants, and mutational signatures are indicated by pattern (see legend). Feature contribution may be due to its presence or absence.

FIG. 5. Calibration of probability scores. Cases were binned according to their re-calibrated probabilities of the associated cancer type predictions (x-axis), showing strong correlation with empirically observed accuracy of predictions.

FIG. 6. Number of correct and total predictions made within each probability range. Calibrated prediction probabilities from cross-validation were computed for the top prediction for each case in the training set. 43.5% of predictions in the training set have cross-validated probability>0.95, with an empirical accuracy of 96.6% (3273/3388).

FIGS. 7A and 7B. Classification performance for cancers of unknown primary. FIG. 7A: Tumor type prediction probabilities for 141 cancers of unknown primary. The fraction of samples (vertical axis) predicted at or above a given probability (horizontal axis) within each cancer type is shown in comparison to the training cohort (7,000 to 10,000 patients) and validation cohort (10,000 to 15,000 patients). FIG. 7B: Fraction of tumors predicted with probability of at least 95% or at least 50%. Of 19 cases predicted with probability of at least 95%, 11/19 (58%) are predicted as non-small cell lung cancer, all of whom are self-reported current or former smokers.

FIGS. 8A-8C. Prediction of colorectal cancer for a cancer of unknown primary. FIG. 8A: Haemotoxylin and Eosin stain of cytological specimen that was sequenced by MSK-IMPACT, a fine needle aspiration of the left neck supraclavicular lymph node. The molecular profile is shown at right. FIG. 8B: Based on the MSK-IMPACT results, colorectal cancer was predicted with high probability (96%). FIG. 8C: Relative contributions of individual features driving prediction of colorectal cancer.

FIGS. 9A-9D. Molecular re-classification changes therapeutic intervention. FIG. 9A: H&E and IHC stains for two lesions in a 67-year old female with a history of breast cancer: a presumed breast cancer metastasis to the lymph node (right) and the original primary breast cancer (left). Genomic profiles for each indicated tumor are shown below. FIG. 9B: Cancer type prediction probabilities (left) and the relative contributions of individual features (right), suggesting a revised classification of lung cancer. Mutations with contributions to classification at the gene-level and alteration type-level (hotspot, truncating) are indicated by two colors proportional to the relative importance of each feature category. FIG. 9C: H&E and IHC stains for two lesions in a 77-year-old female with presumed metastatic lobular breast cancer: a presumed breast cancer metastasis to the bladder (right) and the primary breast biopsy (left). Genomic profiles for each indicated tumor are shown below. PET scans at baseline and after 4 months of treatment with the immune checkpoint inhibitor nivolumab are also shown. FIG. 9D: Cancer type prediction probabilities (left) and the relative contributions of individual features (right) are displayed as described above, suggesting a revised classification of bladder cancer.

FIGS. 10A-1 to 10K provide predictions by a sample trained predictive model when the model is applied to different subjects in the training dataset according to various potential embodiments. In the tables, with respect to 66 study subjects: “Pred” identifies a prediction (e.g., a predicted tumor type); “Conf” refers to confidence scores corresponding to predictions (ranging from 0 to 1, with zero indicating minimum confidence, and one indicating maximum confidence); “Diff_Pred1Pred2” refers to a difference in the confidence scores of the first prediction “Pred1” and the second prediction (“Pred2”); In FIG. 10G-1 to 10K, “Var” refers to features that contributed to the prediction, and “Imp” refers to the corresponding feature importance in the final prediction.

FIG. 11 depicts a block diagram of a server system and a client computer system in accordance with an illustrative embodiment.

### DETAILED DESCRIPTION

For purposes of reading the description of the various embodiments below, the following descriptions of the sections of the specification and their respective contents may be helpful:

Section A describes systems and methods of predicting tissue of origin from targeted tumor DNA sequencing.

Section B describes a network environment and computing environment which may be useful for practicing embodiments described herein.

### Definitions

The definitions of certain terms as used in this specification are provided below. Unless defined otherwise, all technical and scientific terms used herein generally have the same meaning as commonly understood by one of ordinary skill in the art to which the present technology belongs.

As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value). As used herein, an “allele” refers to one of several alternative forms of a gene occupying a given locus on a chromosome.

As used herein, the terms “cancer,” “neoplasm,” and “tumor,” are used interchangeably and refer to cells that have undergone a malignant transformation that makes them pathological to the host organism or subject. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, a “chromosome” refers to a discrete threadlike structure of nucleic acids and proteins that carries genetic information in the form of genes. Chromosomes are visible as morphological entities only during cell division. In humans, each chromosome has two arms, the p (short) arm and the q (long) arm. The short and long chromosome arms are separated from each other only by a centromere, which is the point at which the chromosome is attached to the mitotic spindle during cell division. A chromosome contains roughly equal parts of protein and DNA. The chromosomal DNA contains an average of 150 million nucleotides or bases. The 3 billion base pairs in the human genome are organized into 24 chromosomes. All genes are arranged linearly along the chromosomes. Generally the nucleus of a human cell contains two sets of chromosomes: a maternal set and a paternal set. Each set has 23 single chromosomes: 22 autosomes and an X or a Y sex chromosome.

As used herein, “chromosome gain” refers to the duplication of a chromosome or a chromosomal segment (e.g., p (short) arm or q (long) arm) leading to an unbalanced chromosome complement, or any chromosome number that is not an exact multiple of the haploid number (which is 23 in humans).

As used herein, “chromosome loss” refers to the loss of a chromosome or a chromosomal segment (e.g., p (short) arm or q (long) arm) leading to an unbalanced chromosome complement, or any chromosome number that is not an exact multiple of the haploid number (which is 23 in humans).

As used herein, a “deletion” refers to a mutation (or a genetic alteration) in which part of a DNA sequence at a chromosome location is absent or lost compared to that observed in a reference genome. A deletion may occur within a gene or may encompass one or more genes. A “homozygous deletion” refers to the loss of both alleles of a gene within a genome. A homozygous deletion may comprise a partial or complete loss of each copy (maternal and paternal) of the gene sequence.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, “gene amplification” refers to an increase in the number of partial or complete copies of a single gene sequence or several gene sequences at a specific chromosome locus without a proportional increase in other genes. In some embodiments, gene amplifications can result from duplication of a DNA segment that contains a gene through errors in DNA replication and repair machinery. Gene amplification is common in cancer cells, and may cause an increase in the corresponding RNA and protein encoded by the amplified gene(s).

As used herein, “haploid” describes a cell that contains a single set of chromosomes, e.g., a copy of each autosome and one sex chromosome. In humans, gametes are haploid cells that contain 23 chromosomes, each of which represents one of a chromosome pair that exists in diploid cells. The number of chromosomes in a single set is represented as n, which is also called the haploid number (In humans, n=23).

As used herein, a “hotspot” refers to a site at which mutations or recombination events occur with a significantly higher frequency relative to the mutation or recombination rates of other sites within the genome of a subject. A “hotspot allele” refers to an allele in a hotspot region that occurs at a significantly higher frequency relative to other alleles at the same region. Examples of hotspot alleles are described in Chang M T, et al., Cancer Discov. 2018; 8(2):174-183.

As used herein, a “promoter” means a nucleic acid sequence capable of inducing transcription of a gene in a cell. A promoter is implicated in the recognition and binding of polymerase RNA and other proteins involved in transcription. Promoters may be constitutive, inducible, tissue-specific, ubiquitous, heterologous or endogenous.

As used herein, “signatures” refer to combinations of mutation types that are generated by different mutational processes. Signatures may be derived based on the analysis of whole genome sequences of thousands of tumors (See e.g., Alexandrov L B et al., Nature. 2013; 500(7463):415-421). Different signatures are identified based on the observed substitution classes (e.g., C>A, C>G) and the immediate flanking nucleotides (e.g., ACA>AAA, ACC>AAC). For example, for each tumor profile with a sufficient number of mutations, the observed mutations are compared to the known signatures and the dominant signature responsible for the observed profile is determined. In some embodiments, a signature contributes to the large majority of somatic mutations in the tumor class. If multiple mutational processes are operative, a jumbled composite signature is generated. Examples of methods for extracting mutational signatures from catalogues of somatic mutations are described in Alexandrov L B et al., Nature. 2013; 500(7463):415-421.

As used herein, “structural variants” or “SVs” include duplications, inversions, translocations or genomic imbalances (insertions and deletions). In some embodiments, SVs are about 500 bp to >1 kb in size. Commonly known structural variations include gene fusions as well as copy-number variants (whereby an abnormal number of copies of a specific genomic area are duplicated in a region of a chromosome).

As used herein, the terms “subject,” “individual,” or “patient” are used interchangeably and refer to an individual organism, a vertebrate, a mammal, or a human. In certain embodiments, the individual, patient or subject is a human.

As used herein, “truncation” refers to the premature termination of a polypeptide due to the presence of a termination codon in the sequence of its corresponding structural gene as a result of a nonsense mutation, a frameshift mutation, or a splice site mutation.

As used herein, “variant of unknown significance” or “VUS” refers to an allele, or variant form of a gene, which has been identified through genetic testing, but whose significance to the function or health of an organism is not known.

A. Systems and Methods of Predicting Tissue of Origin from Target Tumor DNA Sequencing

### Introduction

The clinical management of cancer is largely determined by its site of origin, histopathologic subtype, and stage. Even for patients with tumors harboring a therapeutically sensitizing mutation that can guide molecularly-targeted therapy, clinical responses are often influenced by tumor origin. For example, BRAF V600E mutations are observed in cancers arising from numerous tissue sites, and the likelihood of response to RAF inhibitors varies widely as a function of tumor type. While critical for guiding patient management, histology-based cancer identification remains challenging in many patients, especially in those initially presenting with metastatic poorly differentiated neoplasms where ambiguous or incorrect classification may adversely impact choice of therapy and outcome.

While cancer classification has benefited from thorough immunohistochemical evaluation coupled with high quality cross-sectional imaging, molecular alterations highly indicative of the tumor site of origin may further assist in classifications when such tools fail. Some genomic alterations and mutational signatures are strongly associated with specific individual tumor types such as APC loss-of-function mutations in colorectal cancers, TMPRSS2-ERG fusions in prostate cancers, and a UV-associated mutational signature of C>T substitutions in cutaneous melanomas. For other cancer types, combinations of genomic alterations may commonly co-occur, such as TP53 and CTNNB1 mutations in endometrial cancer. The absence of highly prevalent alterations in a given tumor type, such as KRAS mutations in pancreatic adenocarcinoma and recurrent gene fusions in certain sarcomas, can also provide evidence against that particular prediction or classification. Both common and rare genomic alterations across numerous different cancers may, therefore, guide the inference of tumor origin as an adjunct to existing classification approaches.

The feasibility of tumor type classification from genomic data including mutations, copy number alterations, gene expression, methylation, and nucleosome occupancy may be demonstrated. Moreover, such molecular re-assessment of classifications can lead to a change of therapy. Yet the systematic application of such approaches to prospectively generated clinical sequencing data from often sub-optimal FFPE biopsies and their accuracy when applied to the targeted cancer gene panels most commonly used in the clinic to facilitate treatment selection remain largely unexplored.

Here, a machine learning-based approach is established to infer the probabilities of each common solid tumor type classification based on a broad array of genomic alterations identified by targeted tumor sequencing. To ensure applicability for clinical care, the model may be trained on prospective genomic data from advanced cancer patients. Using a population-scale approach allowed us to account for the varying prevalence and co-occurrence of genomic features across all tumor types. The probabilistic genome-based tumor type prediction, when considered alongside traditional immunohistochemical and clinical evaluation, can enable improved predictive accuracy, with important therapeutic implications.

### Methods

### Subjects

The training dataset was derived from a clinical cohort. Patients with rare cancer types or low tumor content were excluded from analysis, resulting in a total training dataset of patients identified or known to have one of 22 cancer types (Table 1). In various embodiments, cancer types may be deemed rare if, for example, they are not among the 50, 40, 30, 25, 20, 15, or 10 most common cancer types. An additional patients subsequently tested by MSK-IMPACT comprised an independent test set. All patients undergoing MSK-IMPACT testing signed a clinical consent form or enrolled on an institutional IRB-approved research protocol (NCT01775072). Demographic characteristics of both cohorts are displayed in Table 2.

### Genomic Analysis

Tumor and matched normal DNAs were sequenced in a CLIA-compliant laboratory using MSK-IMPACT, an FDA-authorized clinical sequencing assay targeting up to 468 key cancer-associated genes. Genomic alterations including mutations, indels, copy number alterations, structural rearrangements, and selected mutation signatures were reported to patients and physicians to guide clinical care and aggregated in a HIPAA-compliant manner in the cBioPortal for Cancer Genomics for further analysis and visualization.

### Random Forest Classifier

As an example technique that may be used in various potential embodiments to predict tumor site of origin, a random forest classifier may be constructed using the training cohort of patients. Prediction accuracy was determined from five-fold cross validation of the training data as well as the independent test set. As many diverse alterations and mutation patterns are associated with different sites of origin, the feature set for classification was drawn from the following categories: mutations and indels (hotspots and gene-level), focal amplifications and deletions, broad copy number gains and losses, structural rearrangements, mutation signatures, mutation rate, and sex. Classifier scores were subsequently calibrated using multinomial logistic regression to match empirically observed classification probabilities.

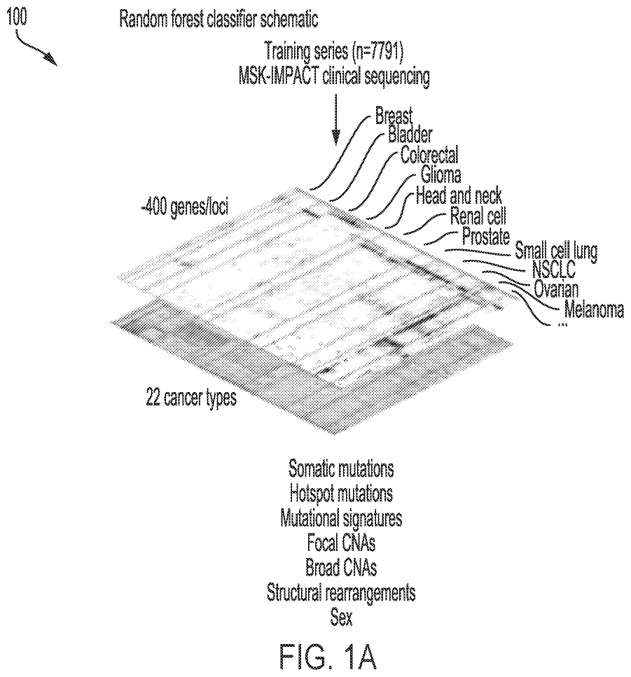

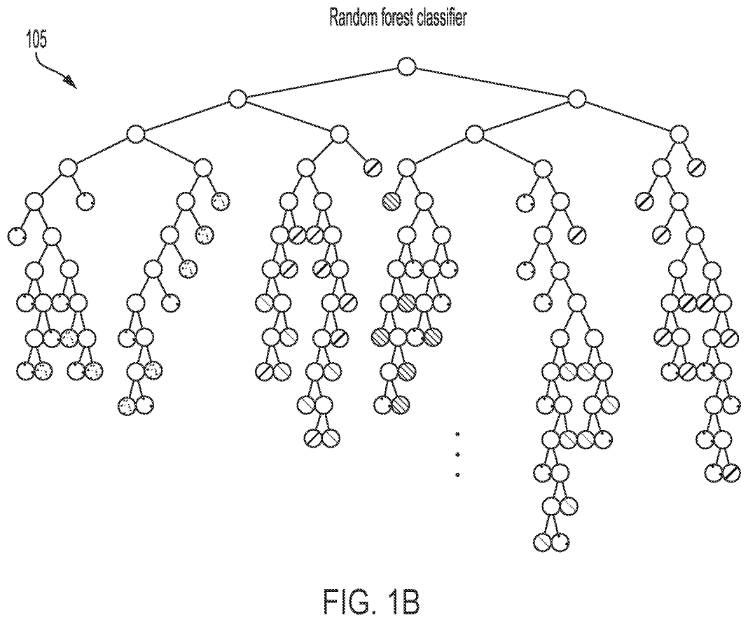

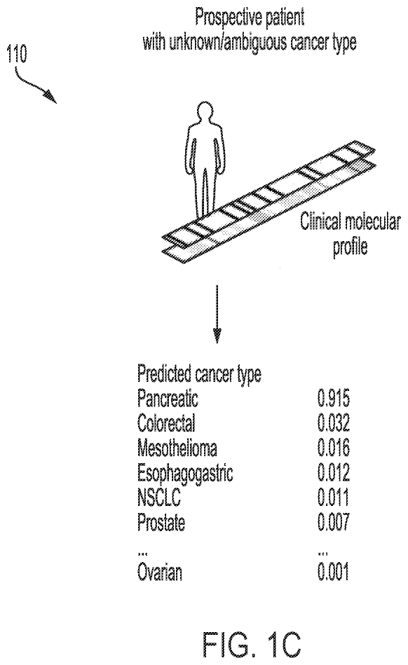

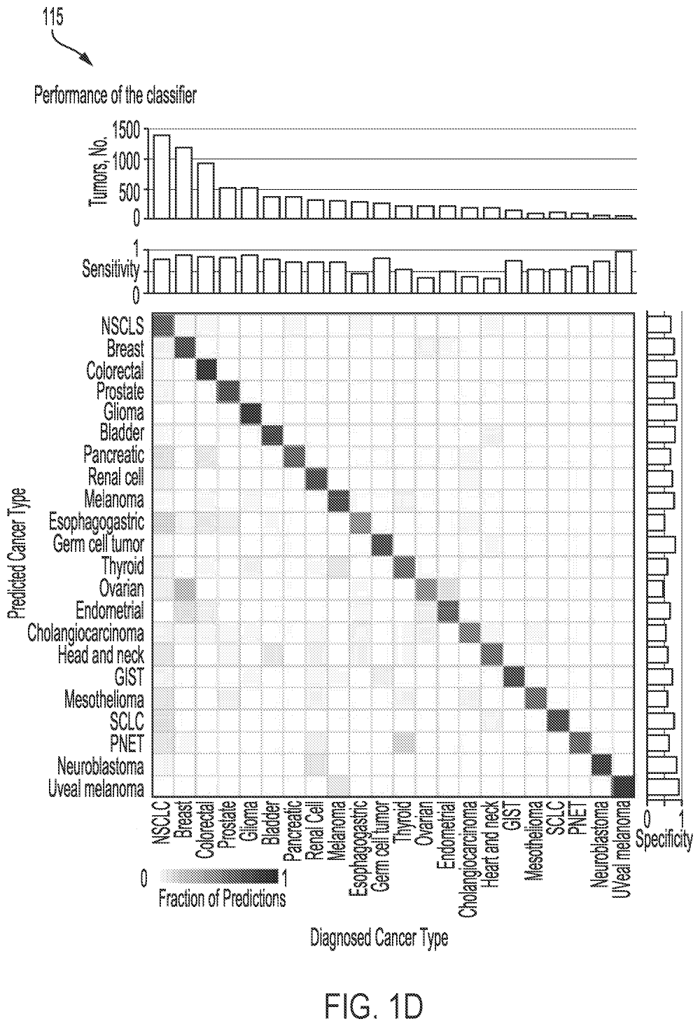

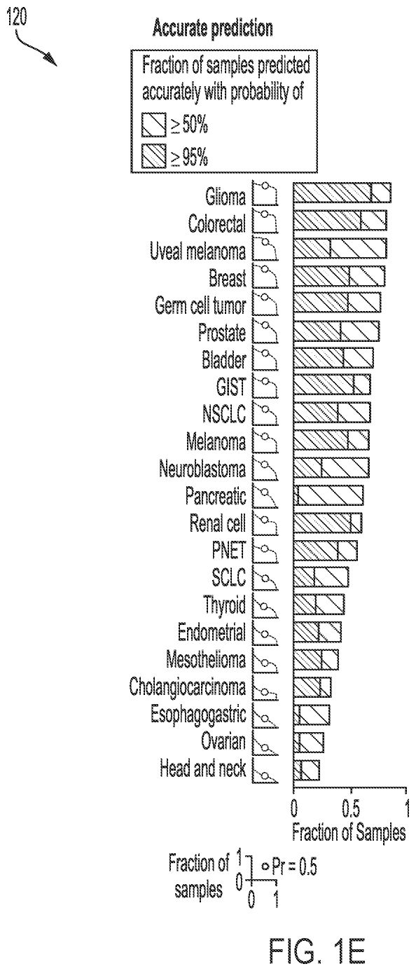

It is hypothesized that the information content from clinical targeted tumor genomic profiling would be sufficiently rich to predict the tumor site of origin with high accuracy. A machine learning-based classifier may be established to determine the ability of DNA genomic alterations (specifically, mutations and indels, focal and broad copy number alterations, structural rearrangements, and mutation signatures) to inform the classification of advanced cancer patients, as depicted in FIG. 1A. Results of the model are detailed herein below in conjunction with FIGS. 1B and 1C.

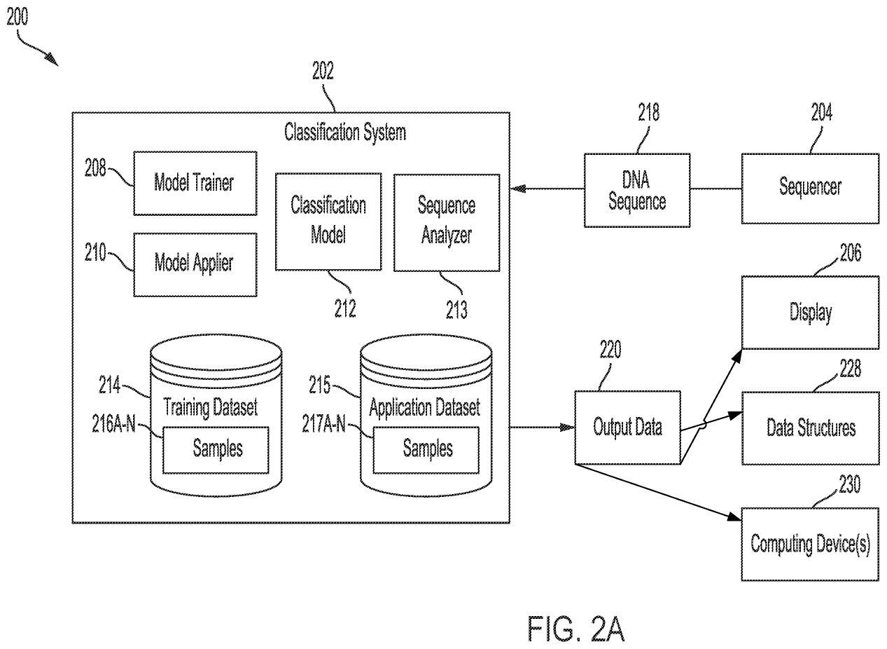

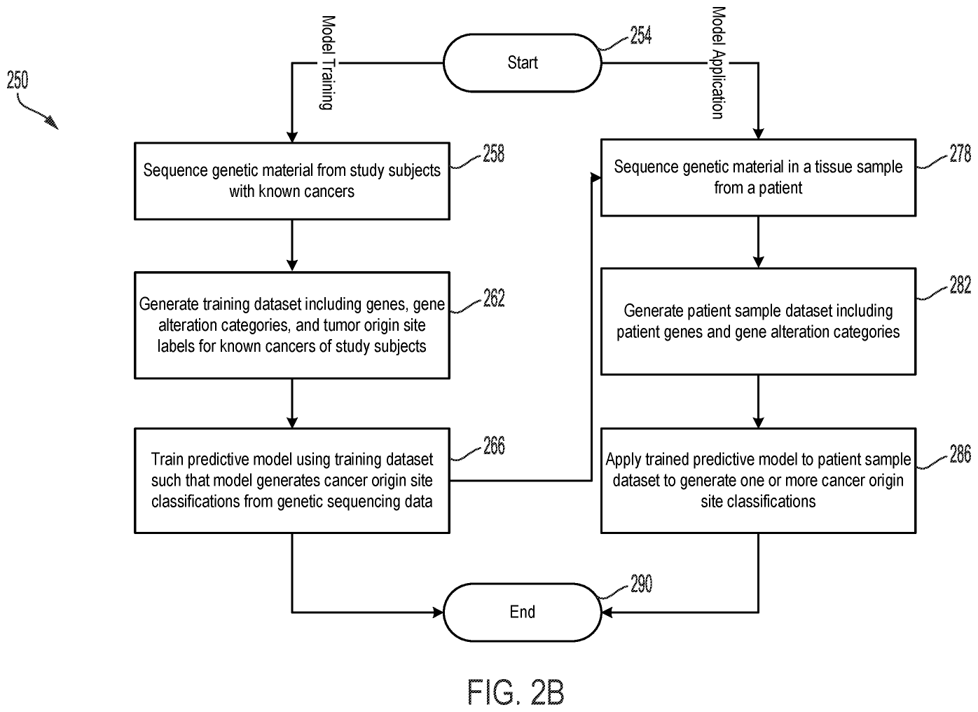

Referring now to FIG. 2A, depicted is a block diagram of a system 200 to determine sites of origin for cancer based on sequencing of genes in accordance with an illustrative embodiment. In overview, the system 200 can include at least one classification system 202 (e.g., a machine learning modeling platform comprising one or more computing devices), at least one sequencer 204, and at least one display 206, among others. The classification system 202 can include at least one model trainer 208, at least one model applier 210, at least one classification model 212 (e.g., a trained predictive model), at least one genetic sequence analyzer 213, at least one training dataset 214, and at least one application dataset 215, among others. The training dataset 214 can be derived from (e.g., by analysis of genetic sequences via sequence analyzer 213) a set of study subject genetic sequence samples 216A-N (training sample datasets). The application dataset 215 can include a set of patient genetic sequence samples 217A-N (patient sample datasets) derived from, for example, analysis (e.g., by analysis of genetic sequences via sequence analyzer 213) of sequences 218 from patients or other subjects. The classification system 202, sequencer 204, display 206, data structures 228, and computing devices 230 can be communicatively coupled to one another.

Each of the components in the system 200 listed above may be implemented using hardware (e.g., one or more processors coupled with memory) or a combination of hardware and software as detailed herein in Section B. Each of the components in the system 200 may implement or execute the functionalities detailed herein in Section A, such as those described in conjunction with FIG. 2A. For example, the classification model 212 may implement or may have the functionalities of the architecture discussed herein in conjunction with FIG. 2A.

The model trainer 208 executing on the classification system 202 may access the training dataset 214 to obtain, retrieve, or otherwise identify training sample datasets 216. The training dataset 214 may have been derived from DNA sequencing (e.g., DNA sequences 218 acquired via sequencer 204) and genetic analysis (e.g., using sequence analyzer 213) of tissue samples from a set of subjects with known cancers. Each DNA sequence sample 216 of the training dataset 214 may record, define, or otherwise include a set of genes, a category, and a label. In various embodiments, particular genes, categories, and labels may be identified and assigned by sequence analyzer analyzing DNA sequences 218. As an example, the set of genes may reference at least some of the genes or alleles described in Table 5. The category may define a nature of alterations to the set of genes of the DNA sequence sample 216. The nature of alterations may include, for example: a gene amplification (AMP), chromosome gain, homozygous deletion, hotspot, allele, chromosome loss, promoter, signature, structural variant (SV), truncation, and variant of unknown significance (VUS), among others. The label may indicate whether the set of genes of the DNA sequence sample 216 is from a cancer subject. In some embodiments, the DNA sequence sample 216 may include one or more traits of the cancer subject, such as sex, age, race and geographic location, among others. The training dataset 214 may be any form of data structure maintainable on the classification system 202, such as an array, a matrix, a table, a linked list, a tree, a heap, and a hash table, among others.

Using the training dataset 214, the model trainer 208 may train, develop, or otherwise establish the classification model 212. In some embodiments, the model trainer 208 may create or instantiate the classification model 212 in response to identifying the training dataset 214. The classification model 212 may be generated, established, and trained in accordance with any number of classification algorithms, such as a linear discriminant analysis, a support vector machine, a regression model (linear or logistic), a Naïve Bayesian classifier, and k-nearest neighbor classifier, among others. In some embodiments, the classification model 212 may be a random forest classifier and the training of the classification model 212 may be in accordance with a random forest algorithm. The classification model 212 may include a set of decision trees (e.g., a classification and regression tree (CART)) to output a likelihood of a presence of cancer at a site of origin given an input DNA sequence. The site of origin may correspond to a type of cancer, and may correspond with an organ in a subject from which the cancer originated, such as a prostate, bladder, breast, and lymph nodes, among others. The random forest classifier, for example, may be selected for its ability to better accommodate large numbers of potentially informative features, arbitrary combinations of features, and the imbalanced class representation of the cohort. The number of decision trees in the random forest classifier may correspond to the number of sites of origins.

To train the classification model 212, the model trainer 208 may perform a bootstrap aggregation process (sometimes referred to as bagging) using the training dataset 214. In performing the process, the model trainer 208 may select random subsets of the DNA sequence samples 216. Each selected DNA sequence sample 216 may include the set of genes, the category, and the label. The number of random subsets may be proportional to the number of sites of origins over the total number of DNA sequence samples 216 in the training dataset 214. In some embodiments, the model trainer 208 may construct or train one of the decision trees in the classification model 212 upon selection of the subsets. The construction of the tree may be in accordance with decision tree learning techniques, such as a classification and regression tree (CART). For example, the model trainer 208 may determine or generate a feature space using the variables in the selected random subset of DNA sequence samples 216. The model trainer 208 may divide the feature space based on where the DNA sequence samples 216 fall, and may construct the tree based on the division of the feature space. Subsequent to the construction, the model trainer 208 may determine a performance metric (e.g., Cohen's kappa) to assess the accuracy and confidence of the tree in the classification model 212.

Once the classification model 212 has been trained or otherwise established, the model applier 210 executing on the classification system 202 can retrieve, receive, or identify at least one patient sample dataset 217 in application dataset 215. The patient sample dataset 217 may comprise or have been derived through genetic analysis (e.g., by sequence analyzer 213) of DNA sequence 218 from the sequencer 204. The sequencer 204 may scan a biopsy sample taken from a subject and perform DNA sequencing to generate the DNA sequence 218, which may be analyzed, for example, by sequence analyzer 213 to identify genes, genetic alterations, etc. (e.g., through comparison of genetic sequences from sequencer 204 with known genetic sequences in a database). The patient or other subject may or may not have cancer. The DNA sequence 218 may include a set of genes and a category. The set of genes may correspond to a particular subset of a DNA sequencing from the tissue sample. The category may define the nature of alteration within the set of genes, such as a gene amplification (AMP), chromosome gain, homozygous deletion, hotspot, allele, chromosome loss, promoter, signature, structural variant (SV), truncation, and variant of unknown significance (VUS), among others. In some embodiments, the DNA sequence 218 may be accompanied by one or more traits, characteristics, or health history of the subject from whom the tissue sample is taken (such as age, gender, smoking history, etc.).

Genetic sequences from the sequencer 204 may be analyzed to generate a patient sample dataset 217, and the model applier 210 may apply the classification model 212 to the patient sample dataset 217. For example, where a random forest classifier is used, the model applier 210 may feed or provide the patient sample dataset 217 as an input to decision trees of the classification model 212. In applying the classification model 212, the model applier 210 may traverse each tree and nodes along at least one path within each decision tree of the classification model 212. By feeding the DNA sequence 218 to each decision tree of the classification model 212, the model applier 210 may generate or otherwise determine a likelihood of a presence of cancer for each site of origin. With the determination, the model applier 210 may send, transmit, or other provide output data 220, which in some embodiments may be provided to display 206 for presentation and/or may be transmitted or otherwise provided to other computing devices 230 or systems via a wired or wireless network communications interface or transceiver. In various embodiments, additionally or alternatively, one or more data structures 228 (which may be stored in classification system 202, in computing device(s) 230, and/or elsewhere) may be generated to comprise the output data 202, or if data structures 228 were previously generated, the output data 220 may be incorporated therein. Data structures 228 may comprise, for example, associations between patients and one or more cancer origin site classifications. The output data 220 may include the set of likelihoods outputted by the classification model 212.

In various embodiments, the training sample datasets 216 may include various other data that may be used to train a predictive model for classifications. For example, in addition to genetic sequence data, the predictive model may be trained using histopathological assessments or other histological data. In various embodiments, the predictive model may be trained by also incorporating other relevant data from the electronic medical records of study subjects.

FIG. 2B illustrates an example process 250 for training a model (e.g., via model trainer 208 of system 202) and/or applying a model (e.g., via model applier 210 of classification system 202) according to various potential embodiments. Process 250 may begin (at 254) by proceeding to model training if there is no trained model, if an existing model is to be further trained, or if training of a new model is to be initiated. At 258, genetic material in samples from study subjects with known cancers may be sequenced (e.g., via sequencer 204) to obtain genetic sequences 218). Genetic sequences may be analyzed (e.g., via sequence analyzer 213) to generate a training dataset at 262. The training dataset may identify genes, gene alterations, and tumor site labels corresponding to known cancers of study subjects.

Using the training dataset, a predictive model (e.g., classification model 212) may be trained at 266. The predictive model may be trained using one or more suitable machine learning techniques, including supervised, unsupervised, or semi-supervised learning techniques. In some embodiments, the predictive model may comprise one or more artificial neural networks. The predictive model may be trained such that it is configured to accept genetic sequencing data (e.g., genes and gene alterations) as input, and generate cancer origin site classifications as outputs. In certain embodiments, process 250 may end (290) after step 266.

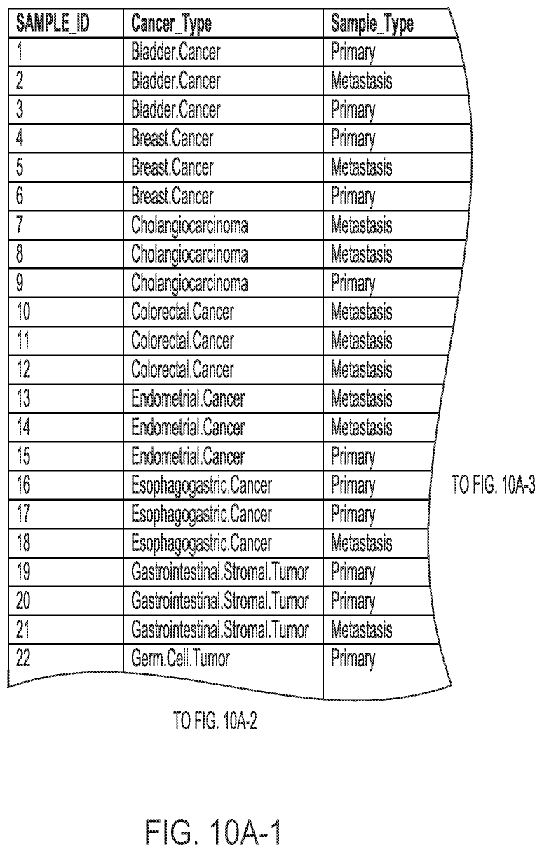

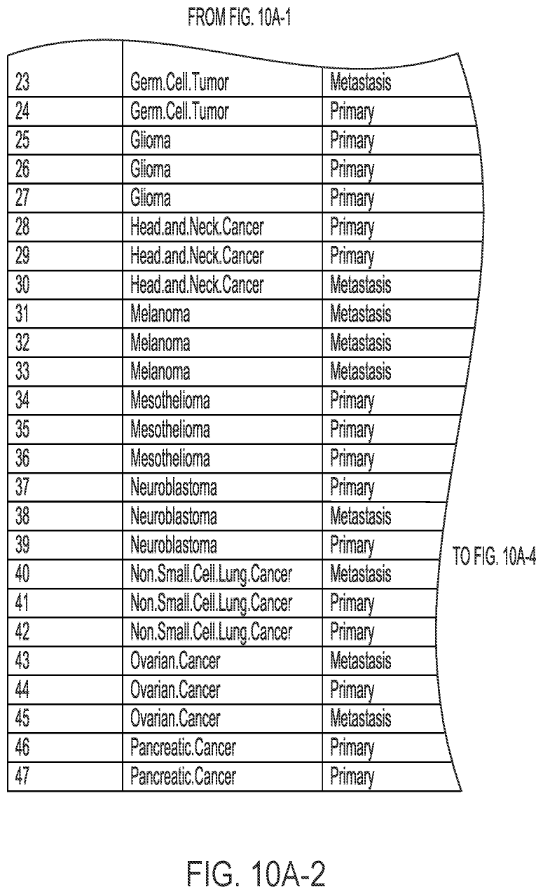

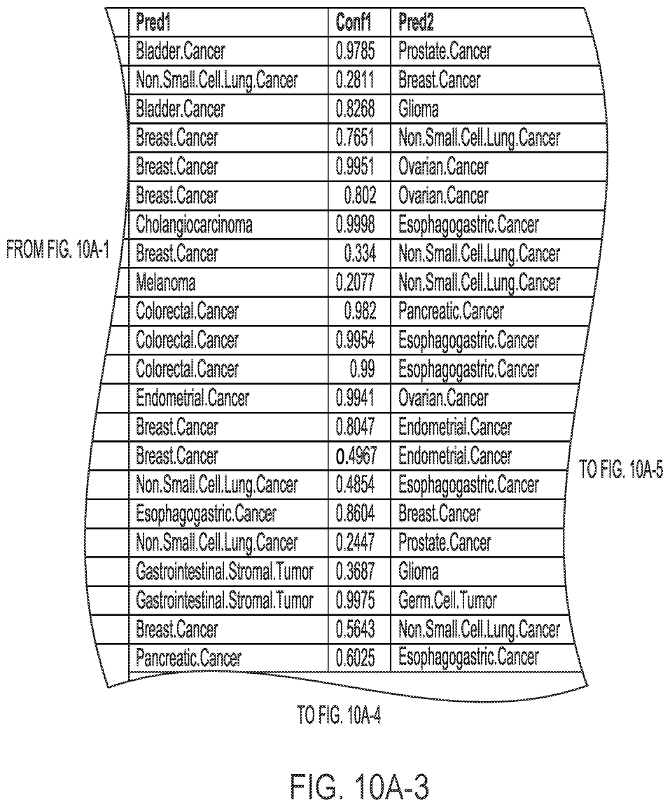

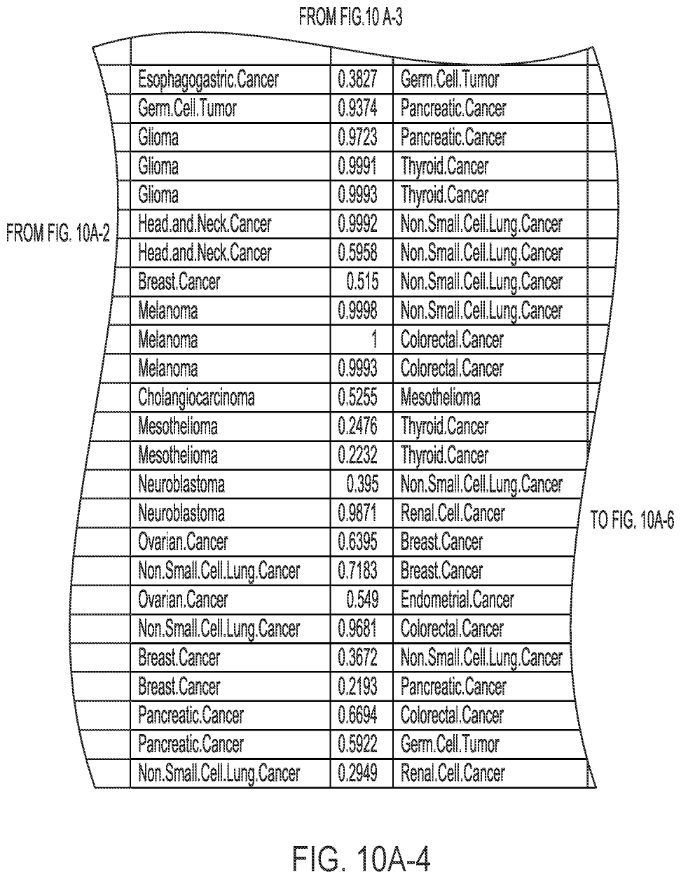

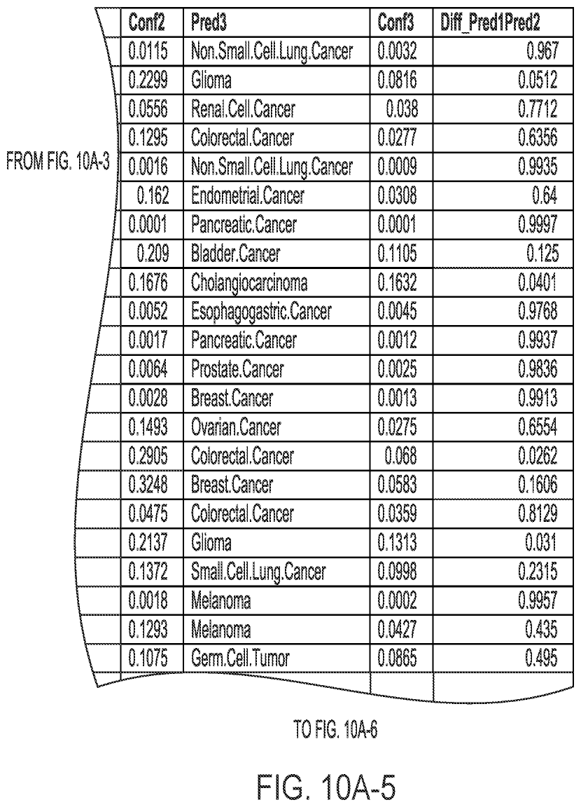

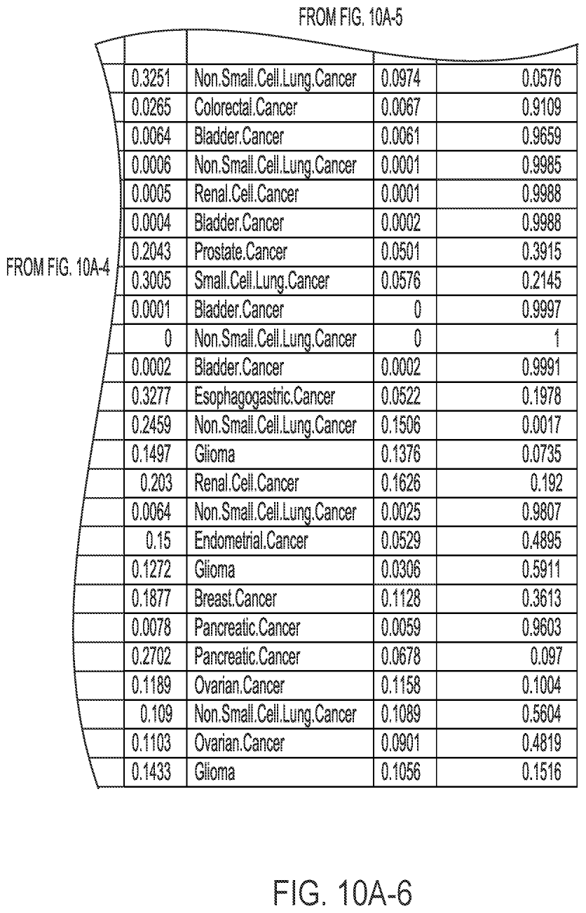

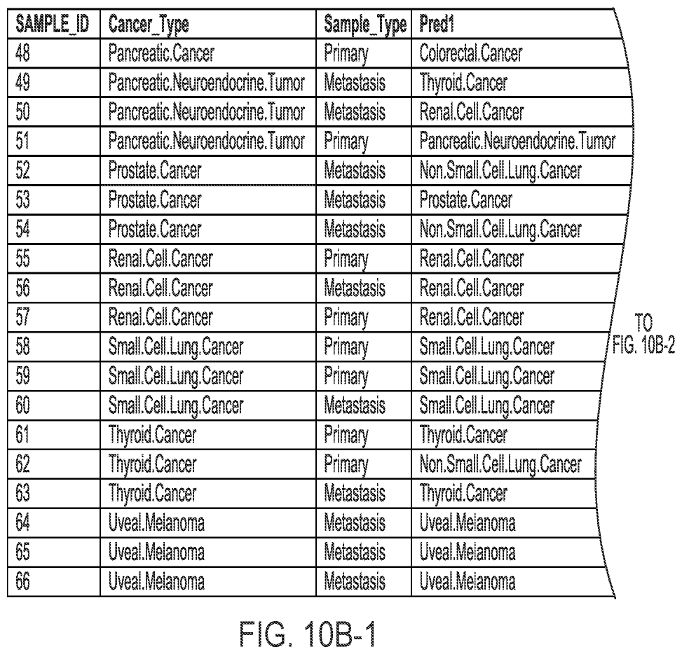

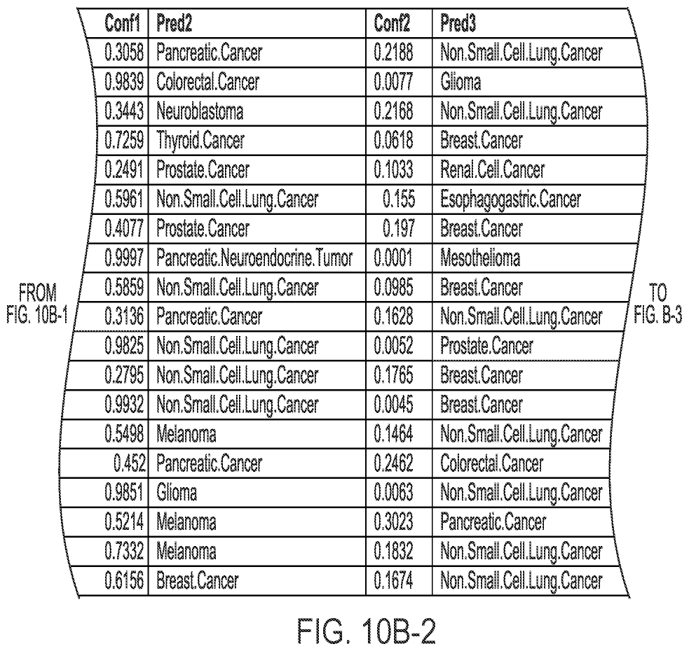

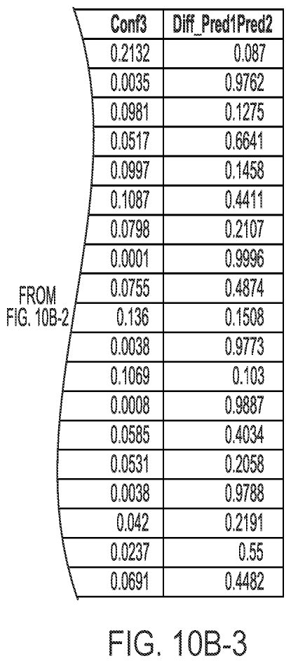

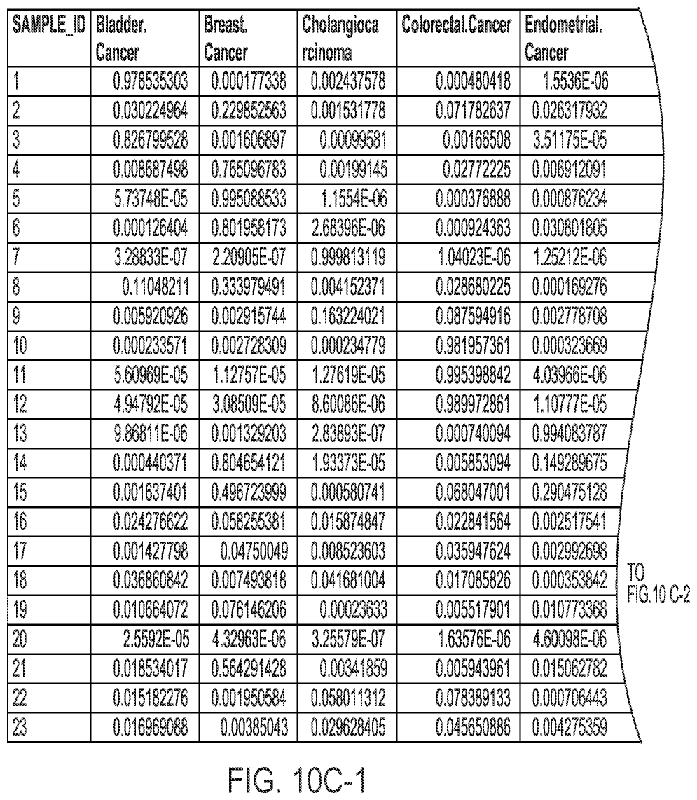

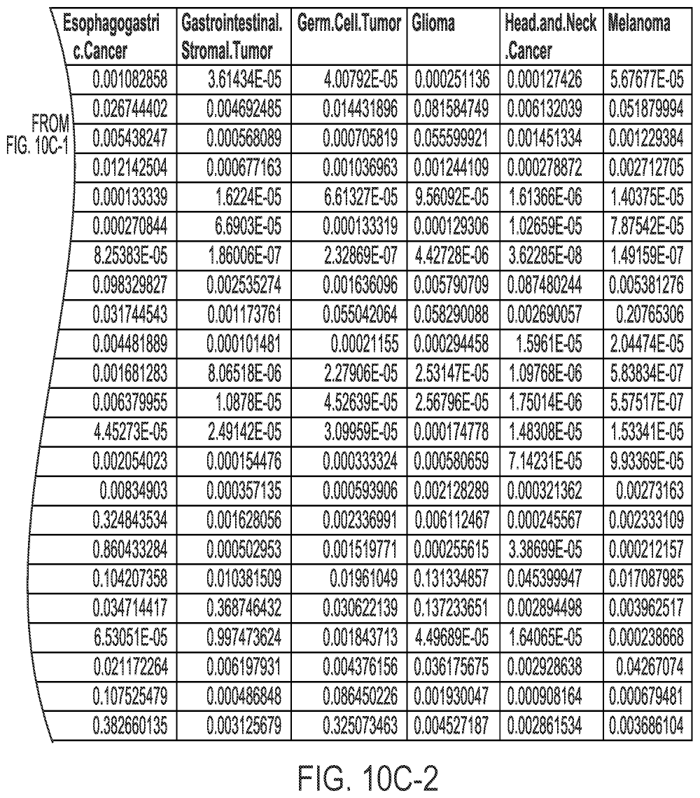

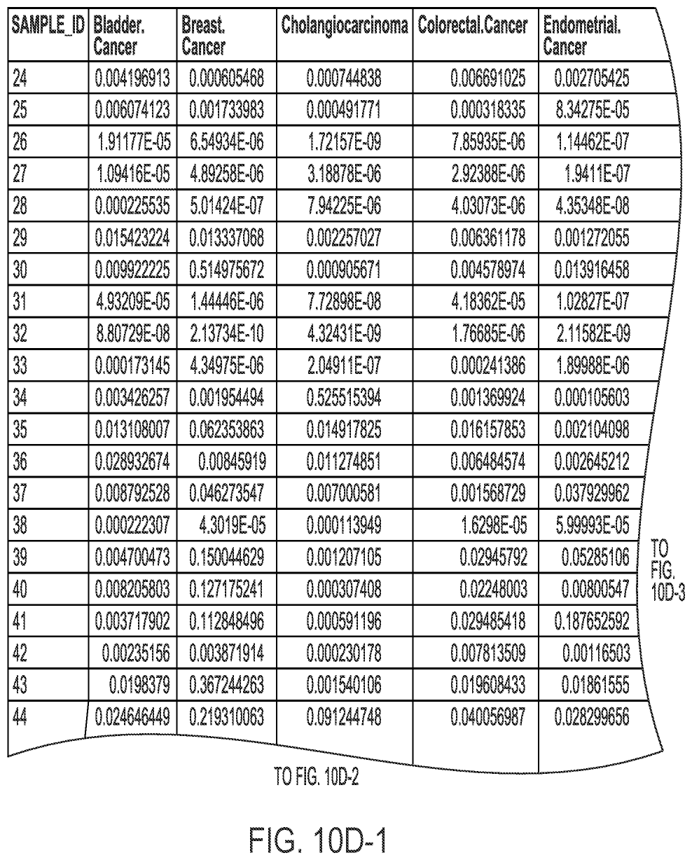

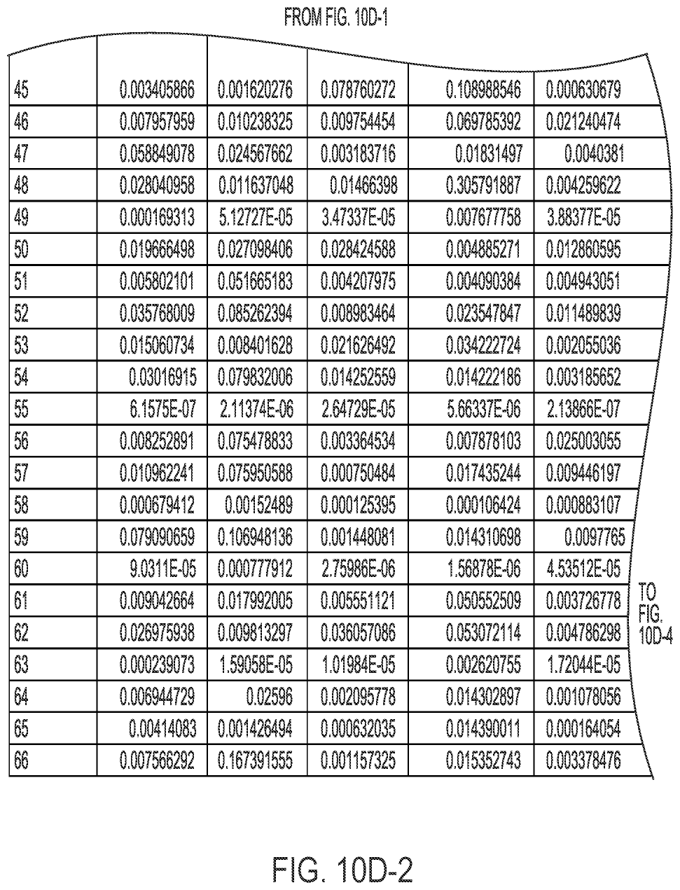

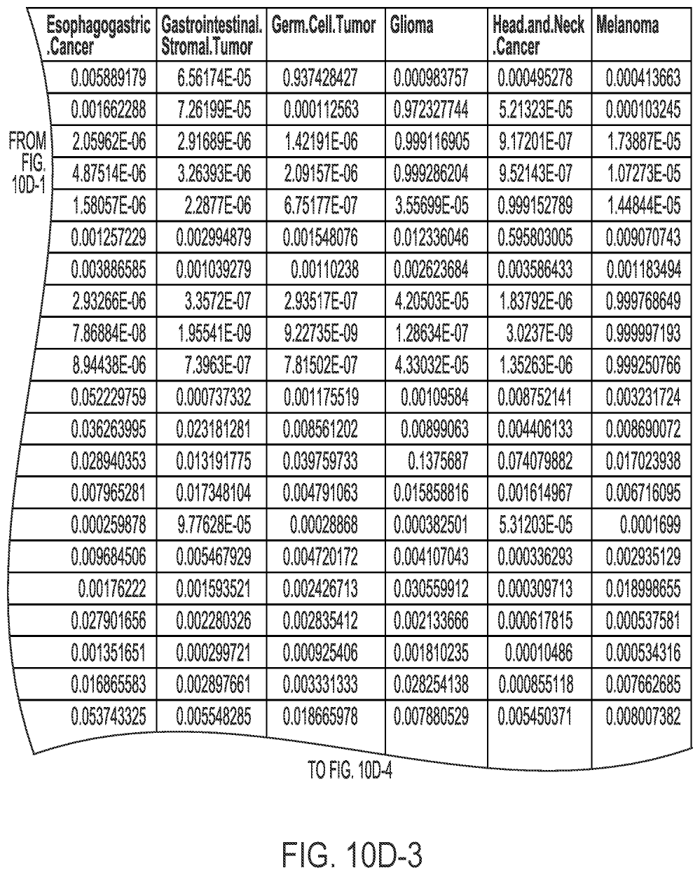

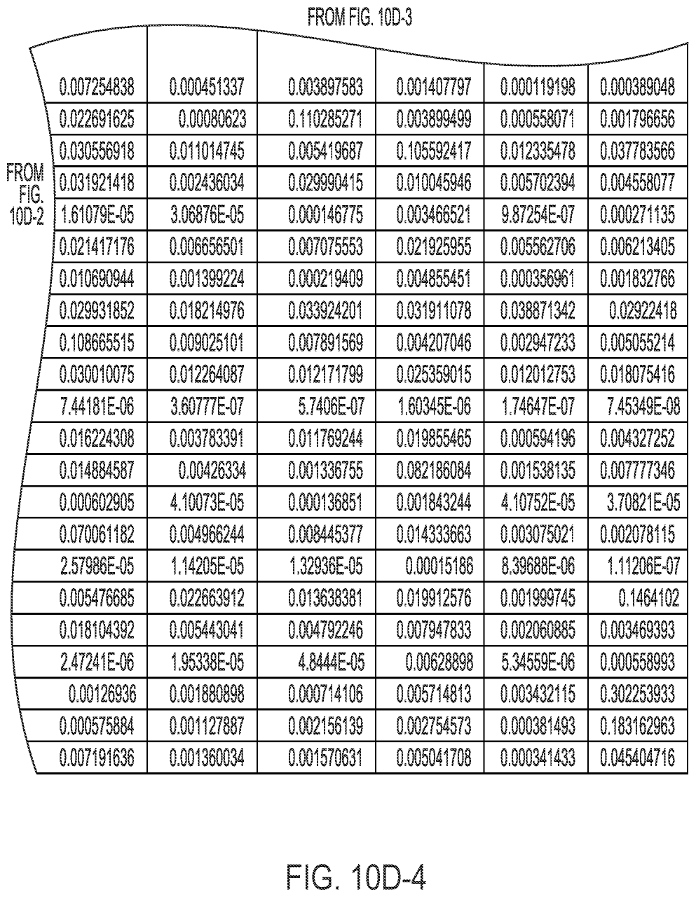

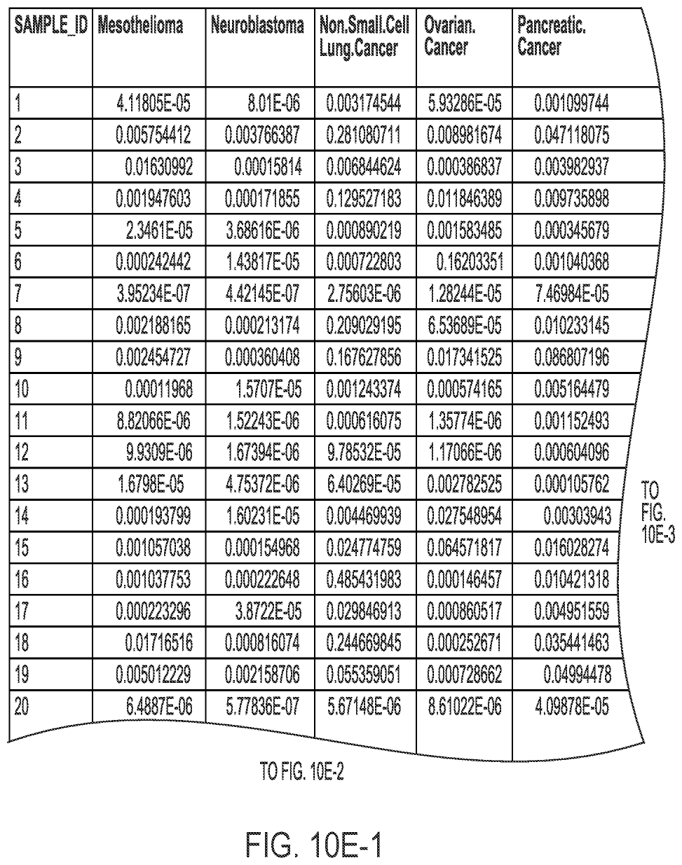

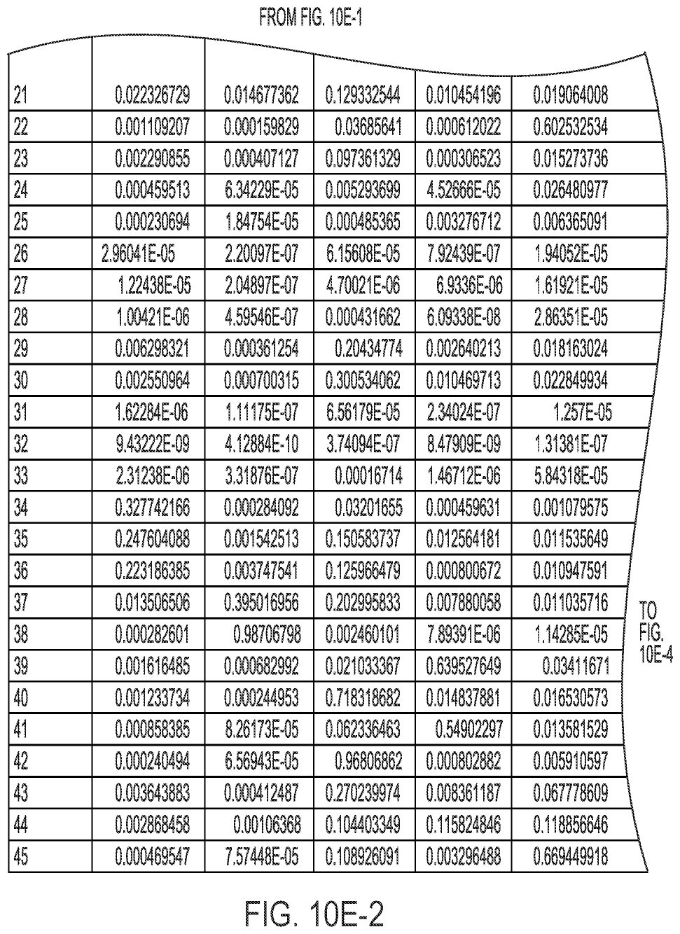

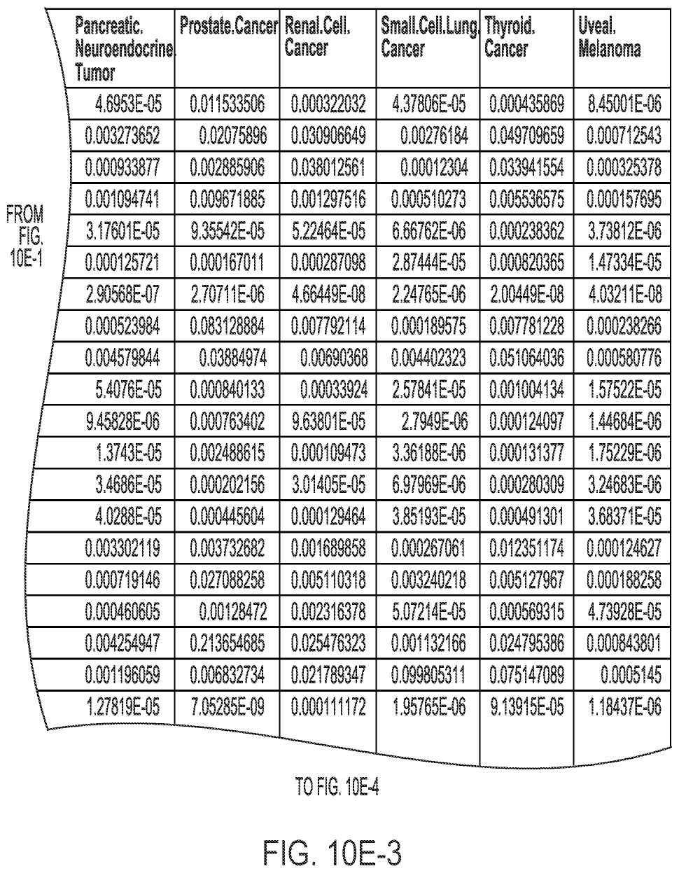

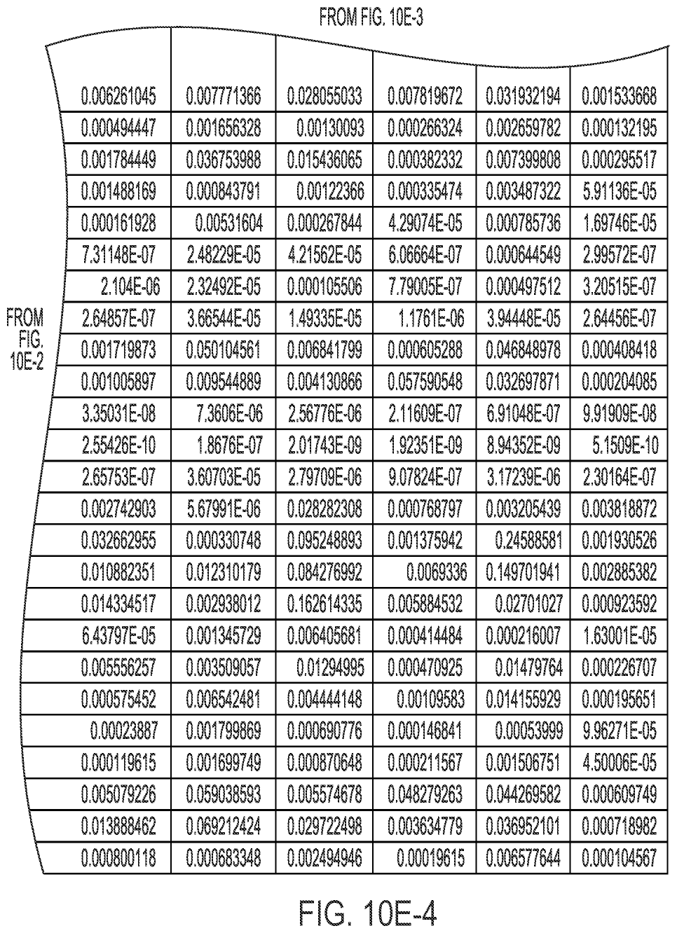

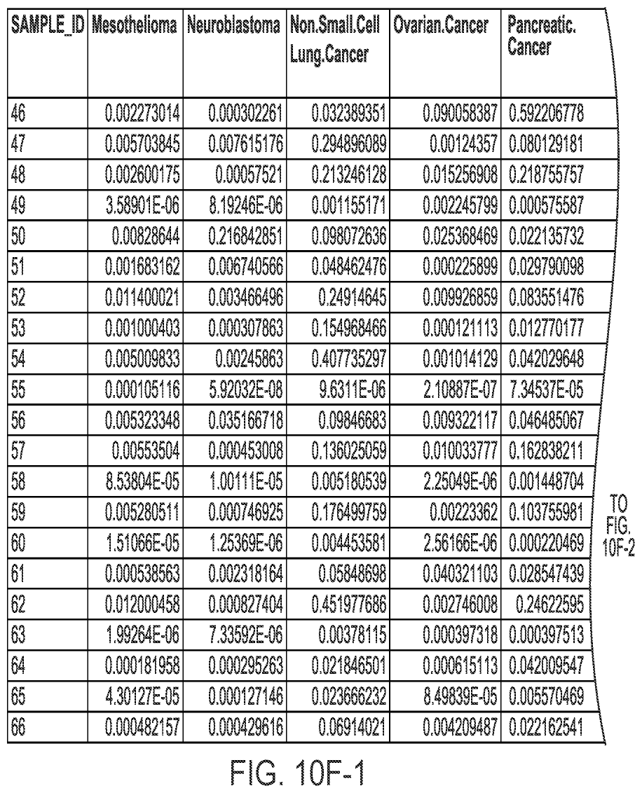

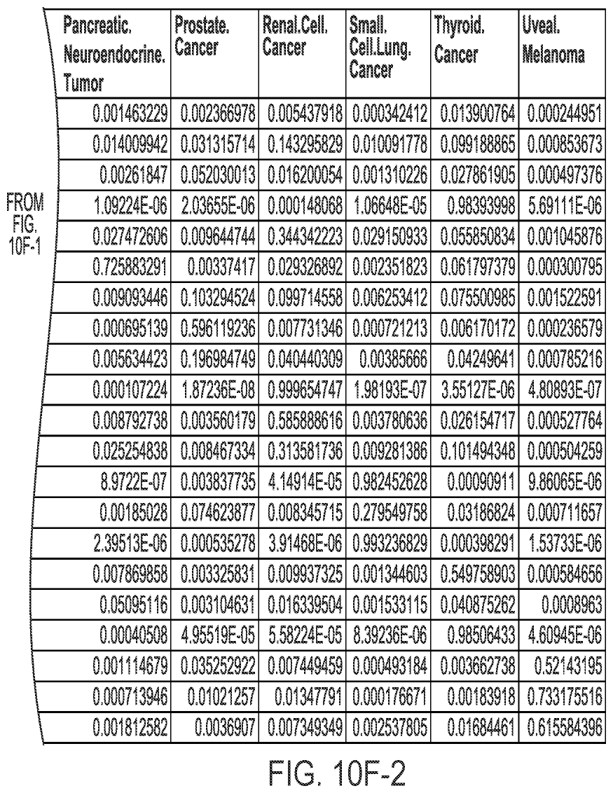

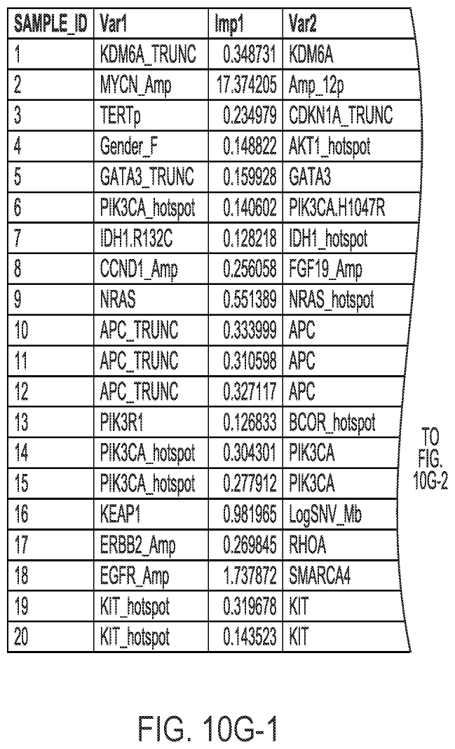

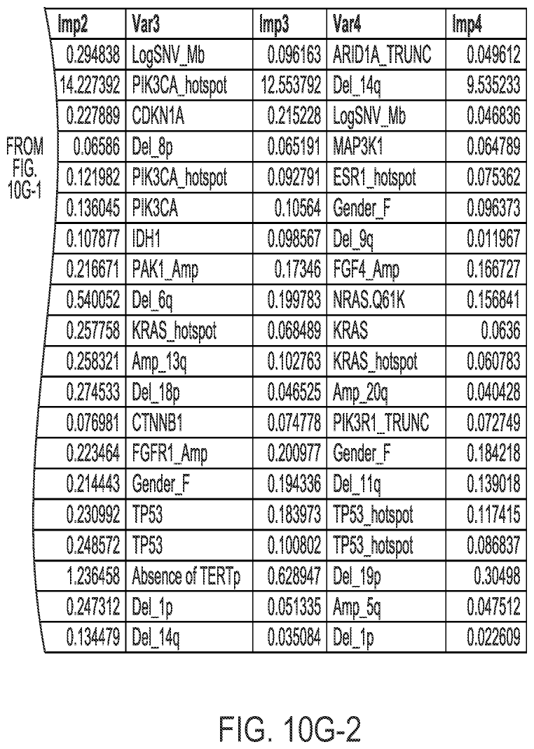

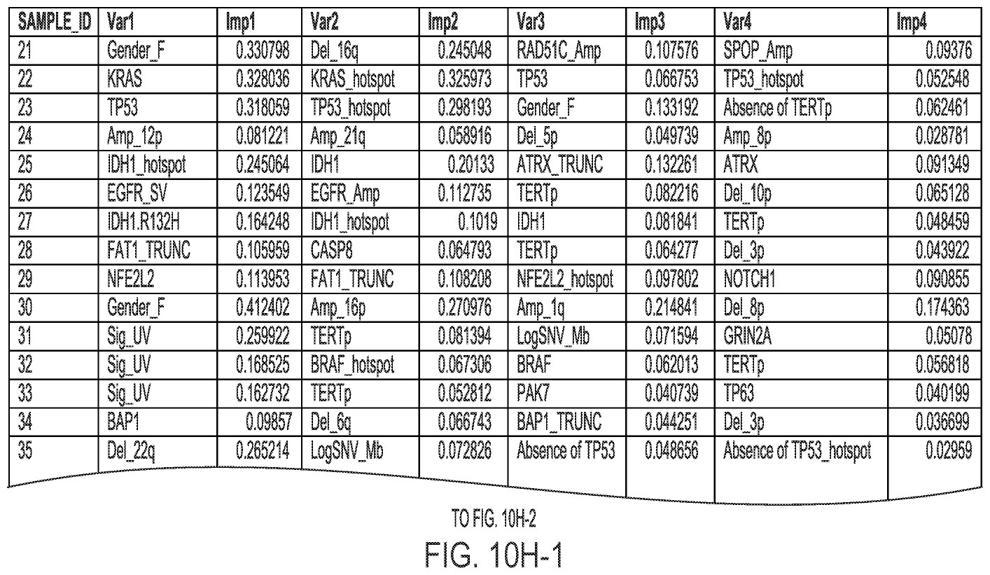

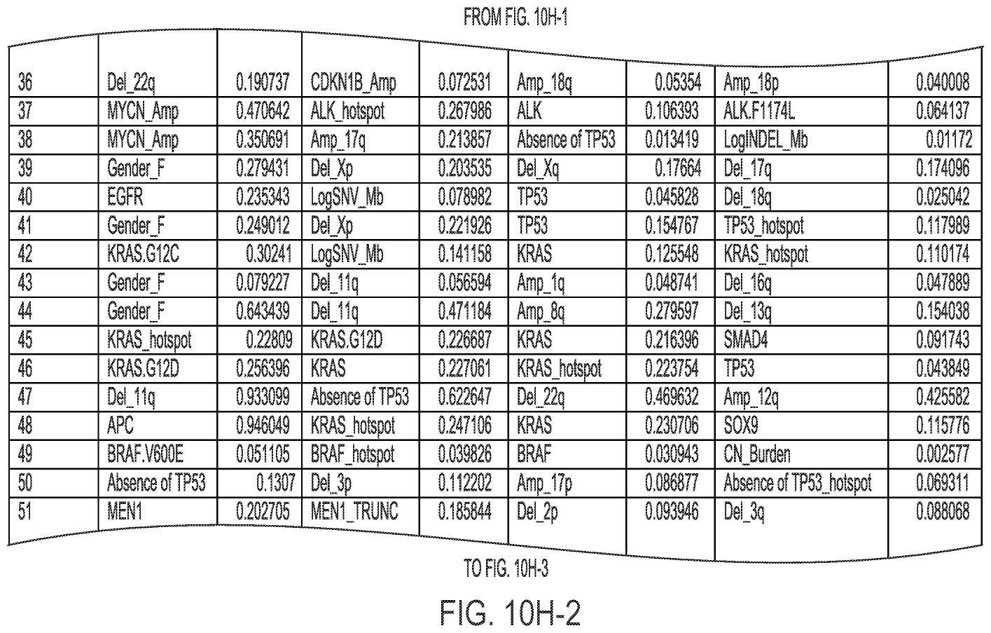

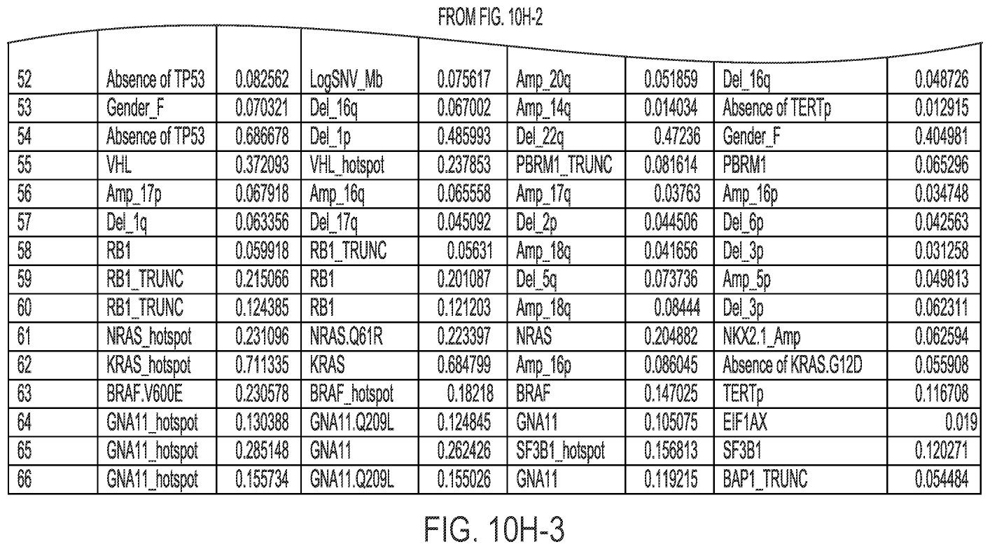

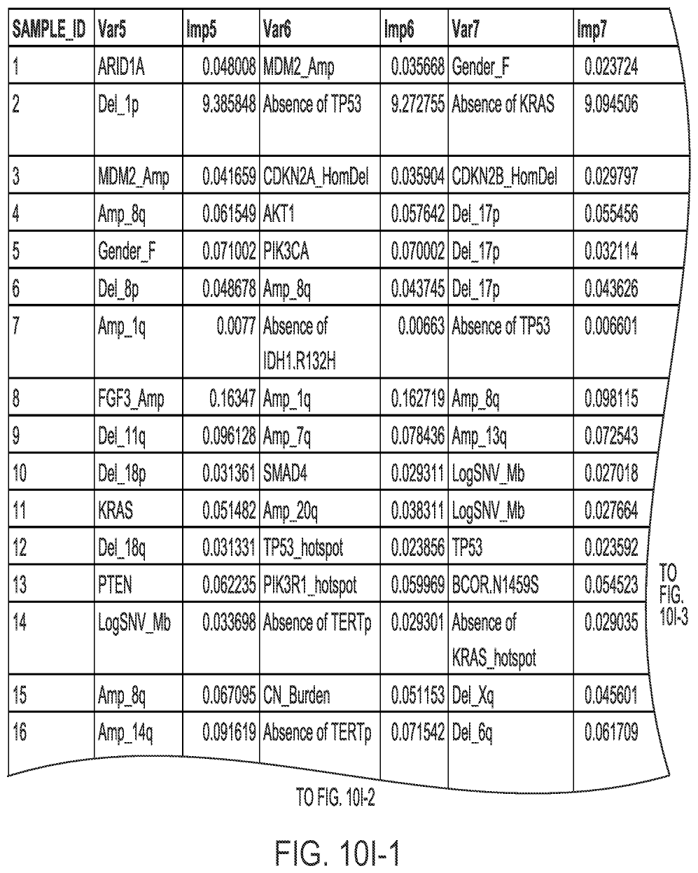

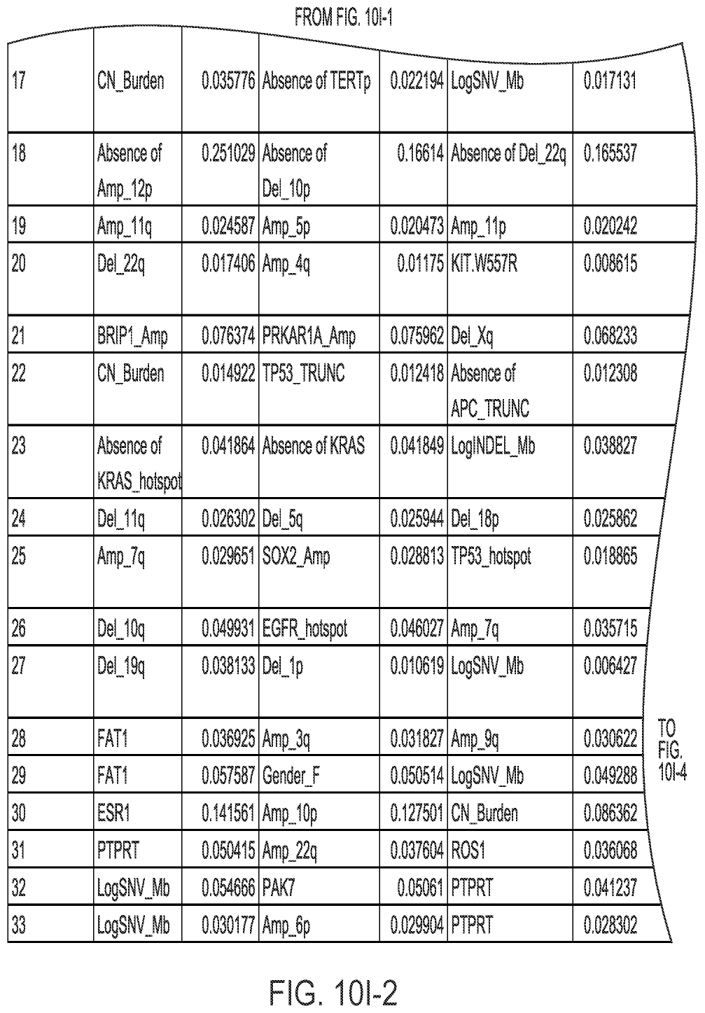

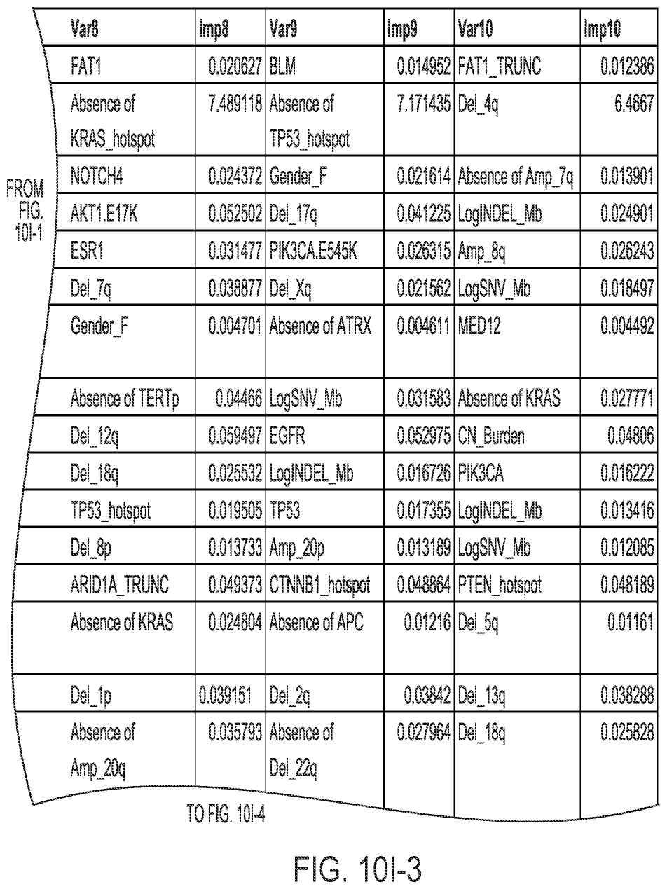

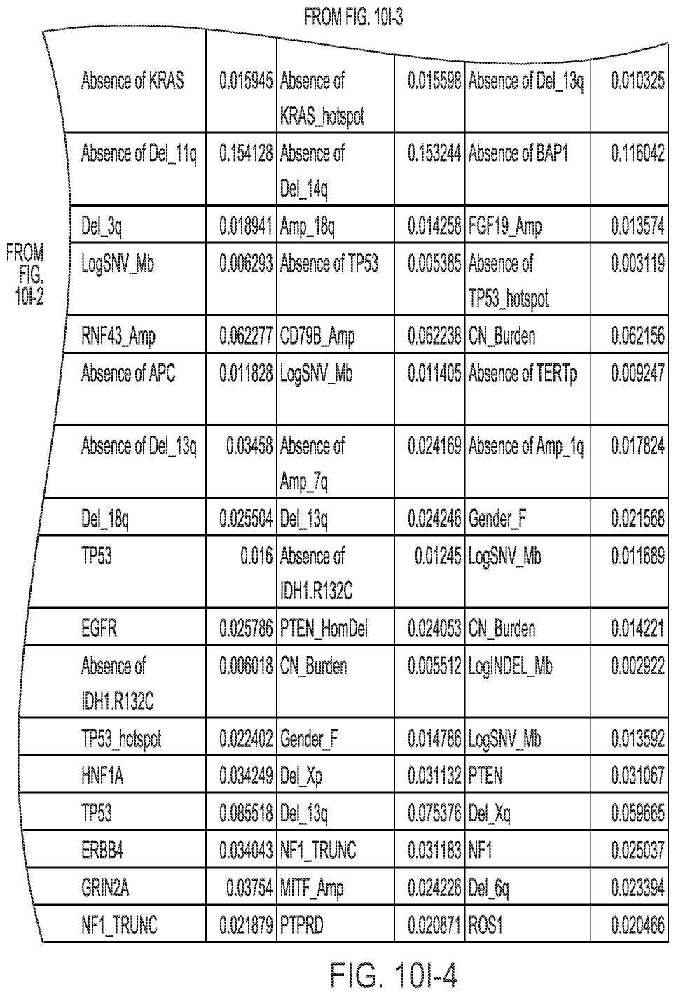

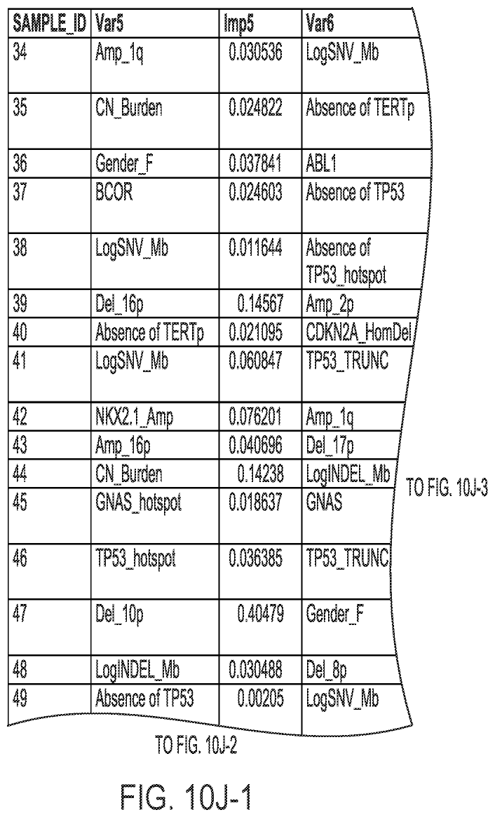

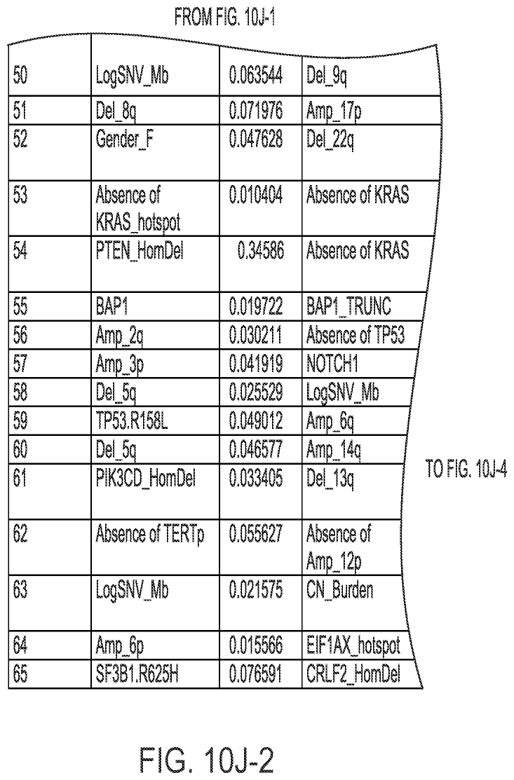

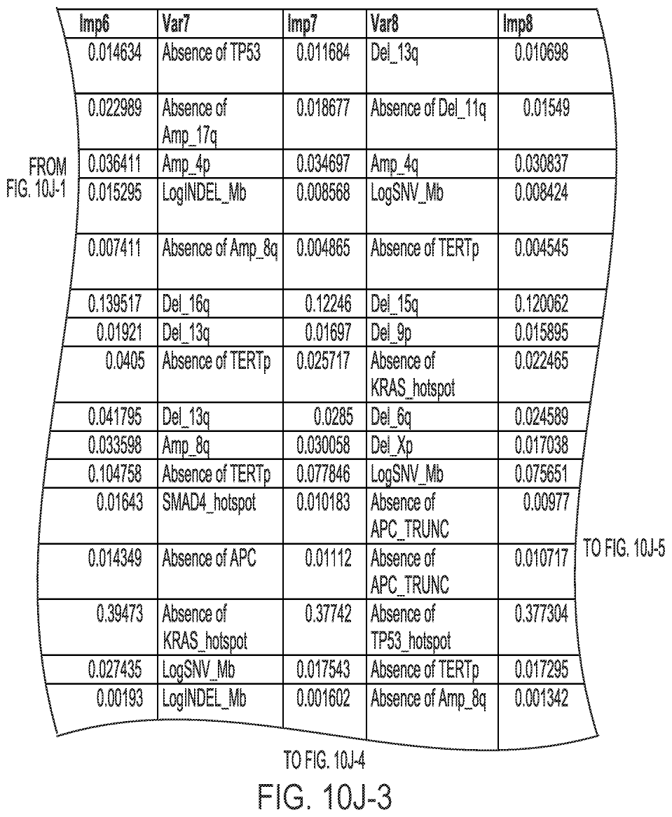

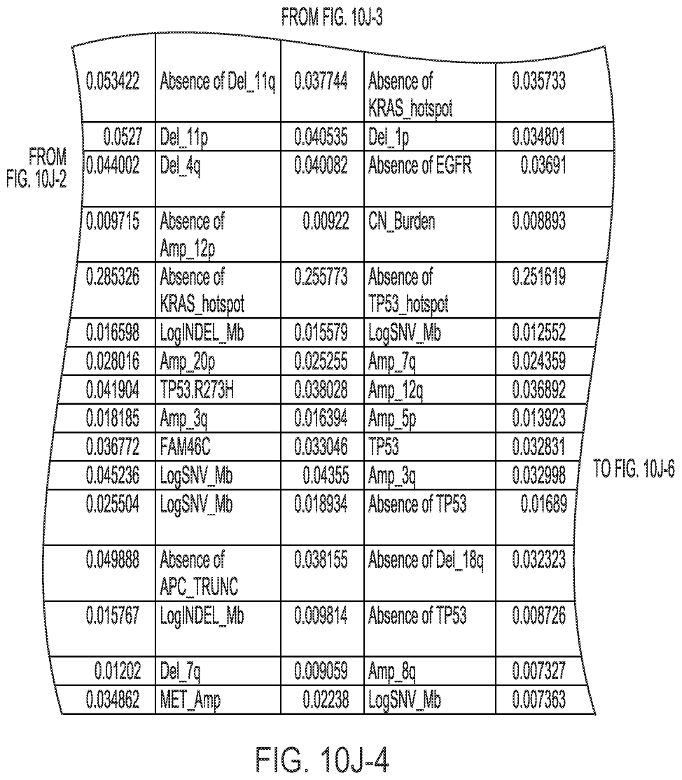

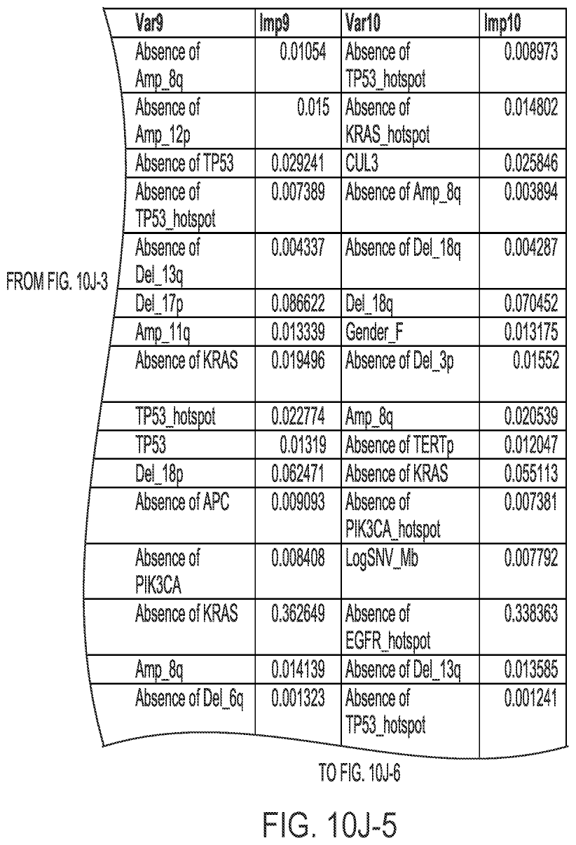

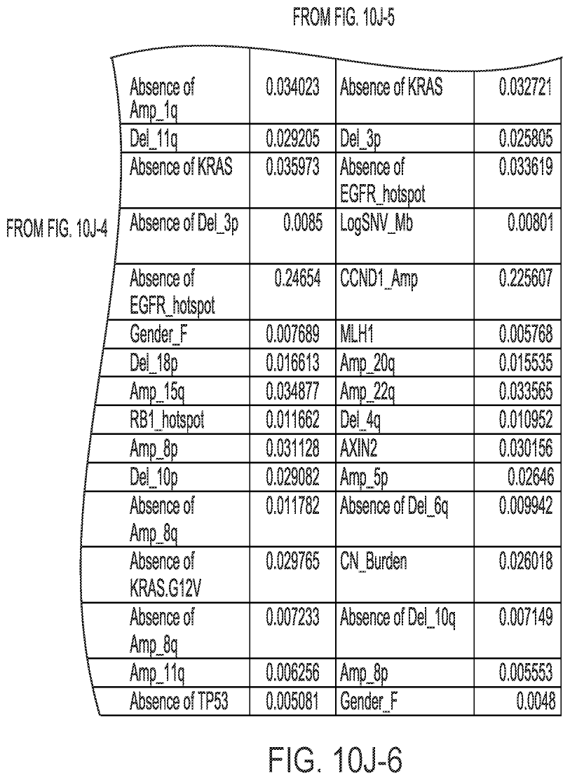

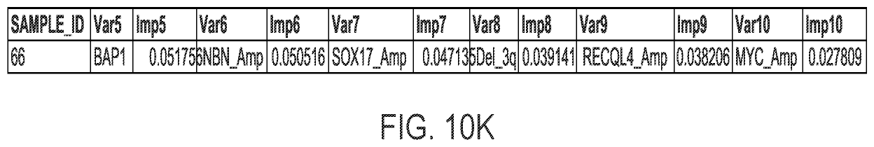

In various embodiments, process 250 may begin (254) by proceeding to model application at 278. In certain embodiments, process 250 may proceed to step 278 following step 266. At 278, genetic material in a tissue sample from a patient may be sequenced (e.g., by sequencer 204 to obtain DNA sequence 218). Genetic sequence data may be analyzed (e.g., by sequence analyzer 213) to identify genes and/or gene alterations. At 282, a patient sample dataset may be generated based on analysis of the sequenced genetic material of the patient. At 286, a trained predictive model (e.g., following step 266) may be applied to the patient sample dataset to generate an output (see, e.g., FIG. 10). For example, the predictive model may generate cancer origin site classifications as output. In various embodiments, the predictive model may output predicted cancer sites (e.g., internal organs and/or systems) and/or cancer types. In various embodiments, the predictive model may additionally generate a likelihood corresponding to each classification (e.g., each organ or each cancer type). The likelihoods may be derived from or may comprise confidence scores output by the predictive model.

The outputs (e.g., output data 220) may, in various embodiments, be displayed (e.g., via display 206) and/or transmitted to other computing devices 230 (e.g., devices of healthcare professionals who may be treating the patient) for further analysis and/or for use in planning treatment or therapeutic protocols. In various embodiments, the output data 220 may be further analyzed (by itself or in combination with other patient data available in, e.g., the patient's electronic medical records) by system 200 to automatically generate one or more treatment or therapeutic recommendations. In certain embodiments, output data 220 may comprise various treatment or therapeutic recommendations. An association between a subject and classifications (e.g., organs, cancer types, and/or confidence scores) may be stored in one or more data structures.

### Performance of Embodiments of Tumor Type Predictive Model

In the training set of patients tested by MSK-IMPACT, in an illustrative embodiment, cancer type was accurately predicted in 73.8% of cases based on five-fold cross-validation (FIG. 1B, Table 3, Appendix). The positive predictive value was highest in tumor types with distinctive molecular profiles such as uveal melanoma (95%), glioma (87%), and colorectal cancer (85%), with predictions driven by diverse sets of genomic features (FIGS. 1A-1C). For other more heterogeneous tumor type categories, prediction accuracy varied among detailed histological subtypes (Table 4). Applying the full classifier 15 to predict the site of origin from MSK-IMPACT clinical sequencing in an independent test set of additional patients, an equivalent accuracy of 74.1% may be observed.

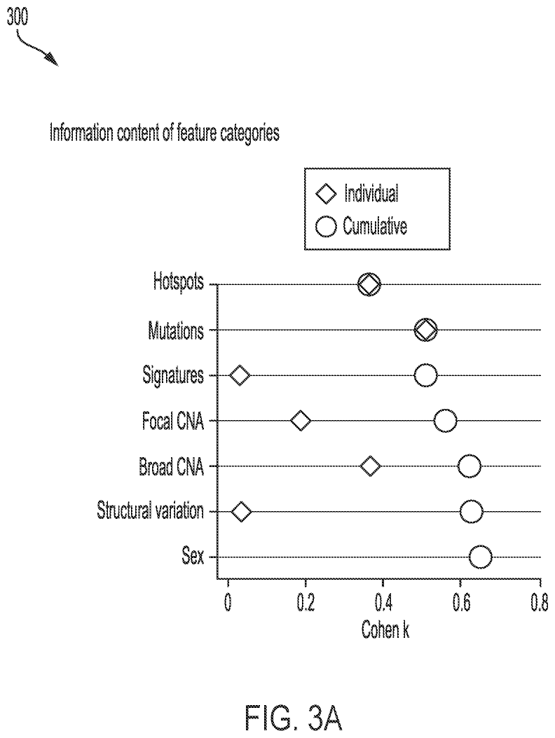

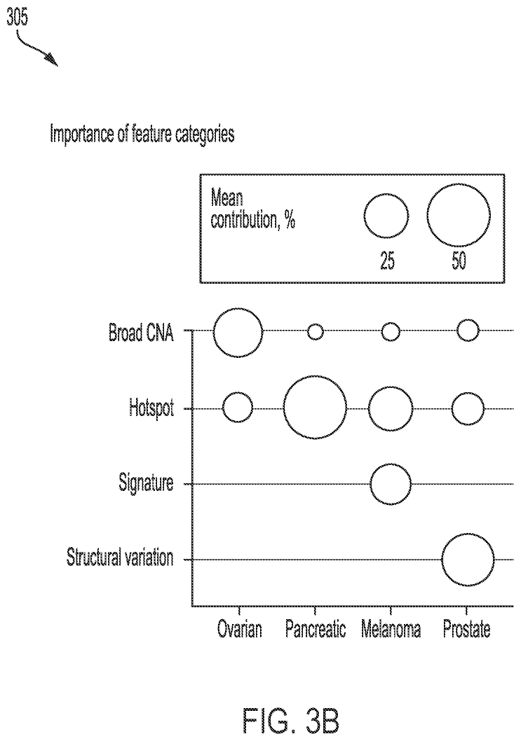

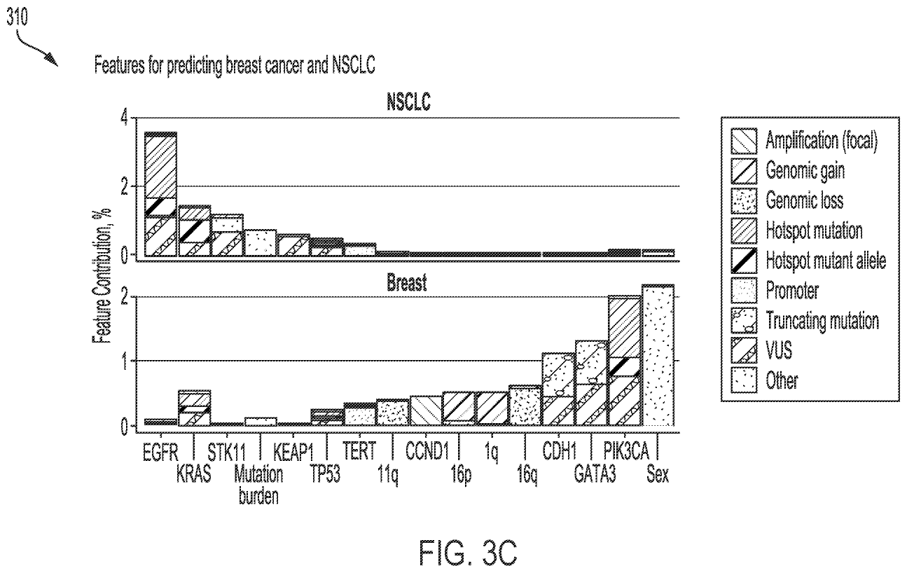

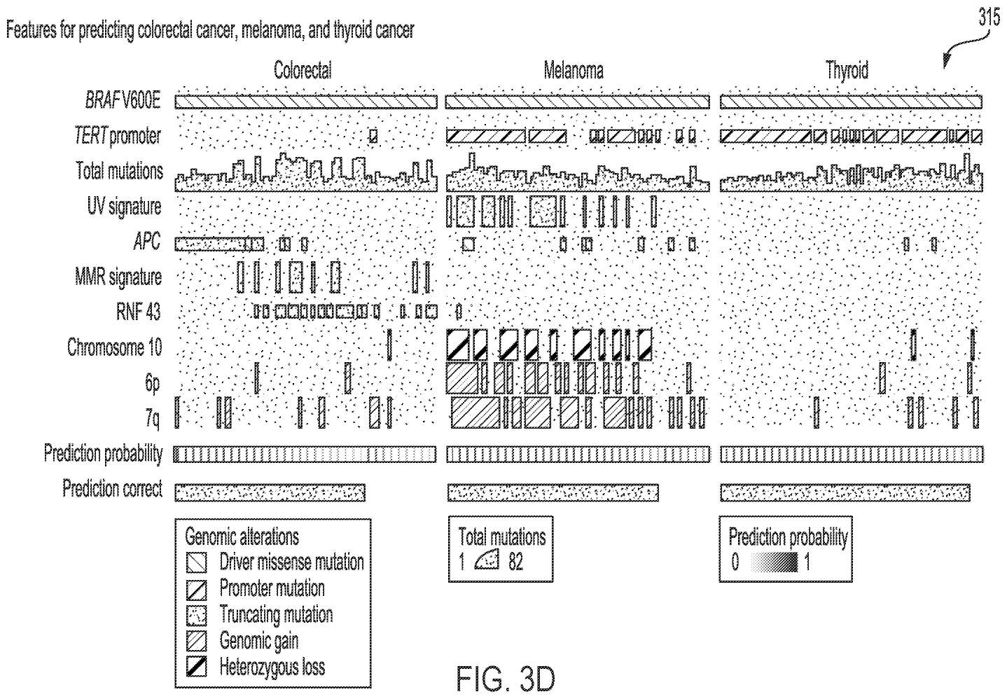

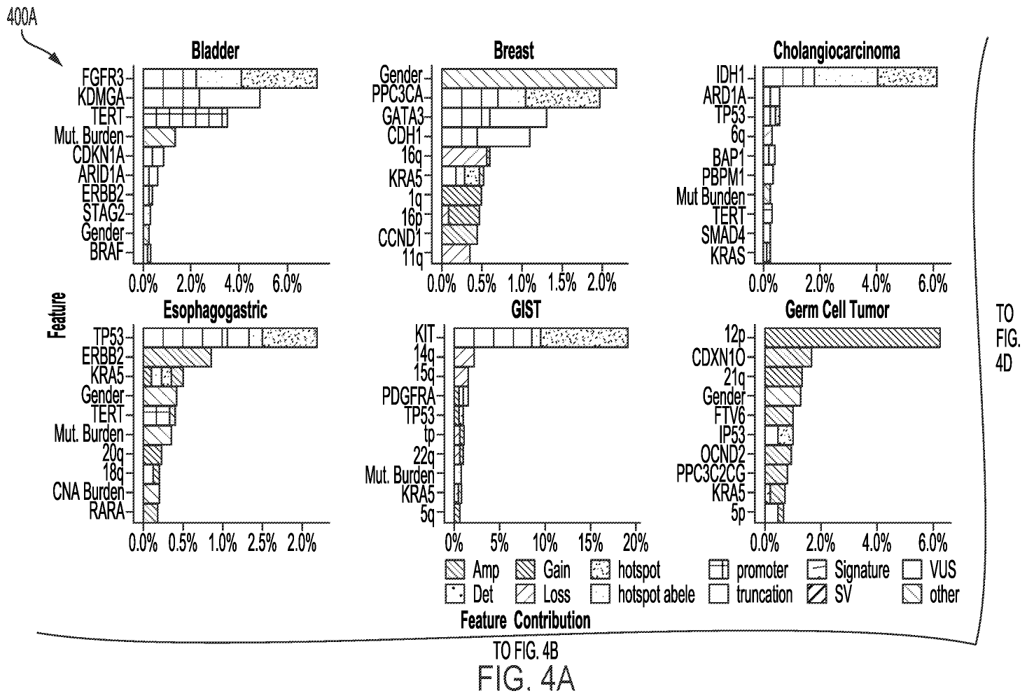

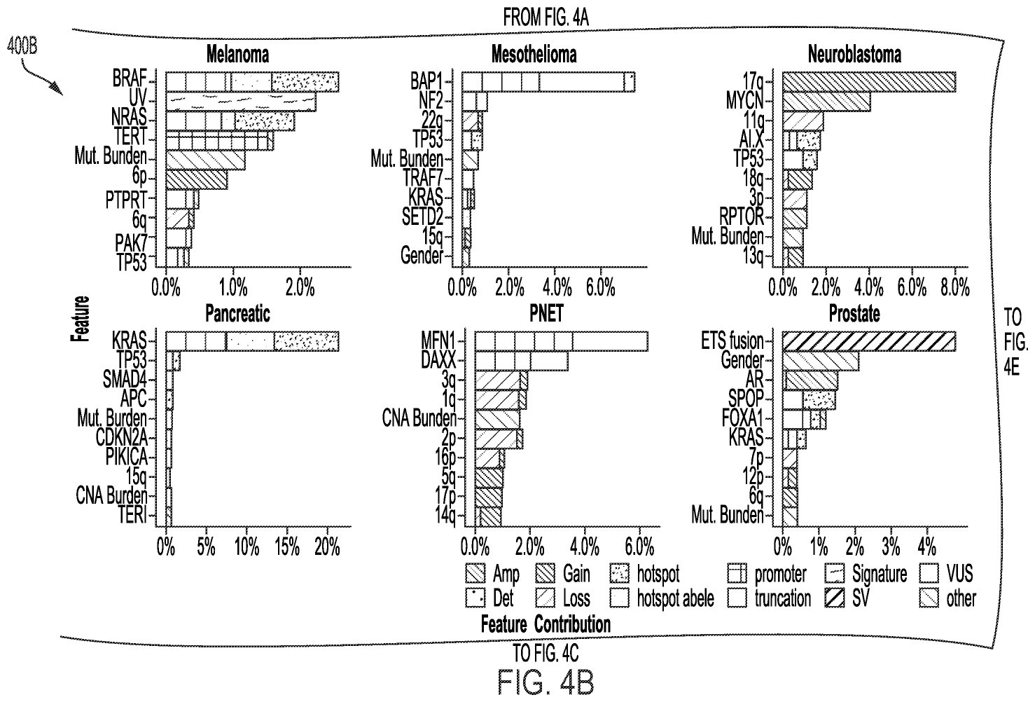

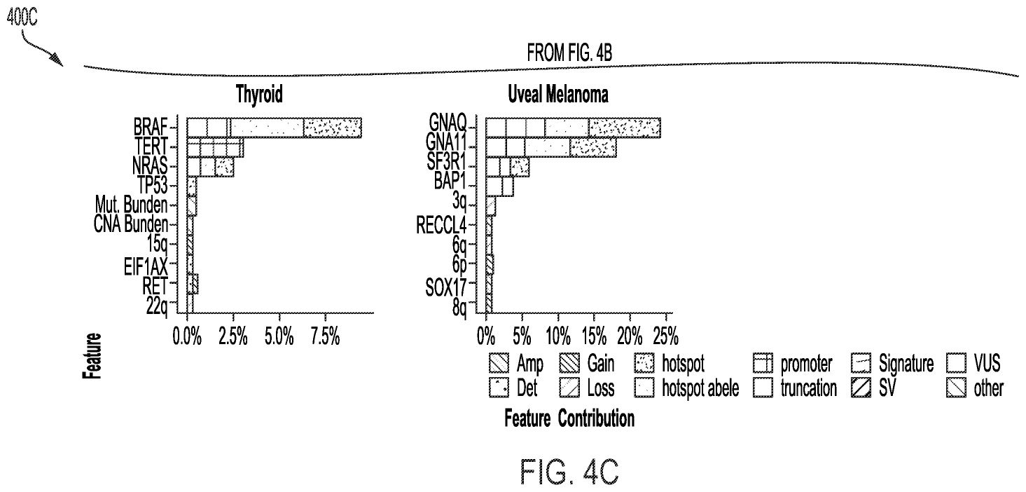

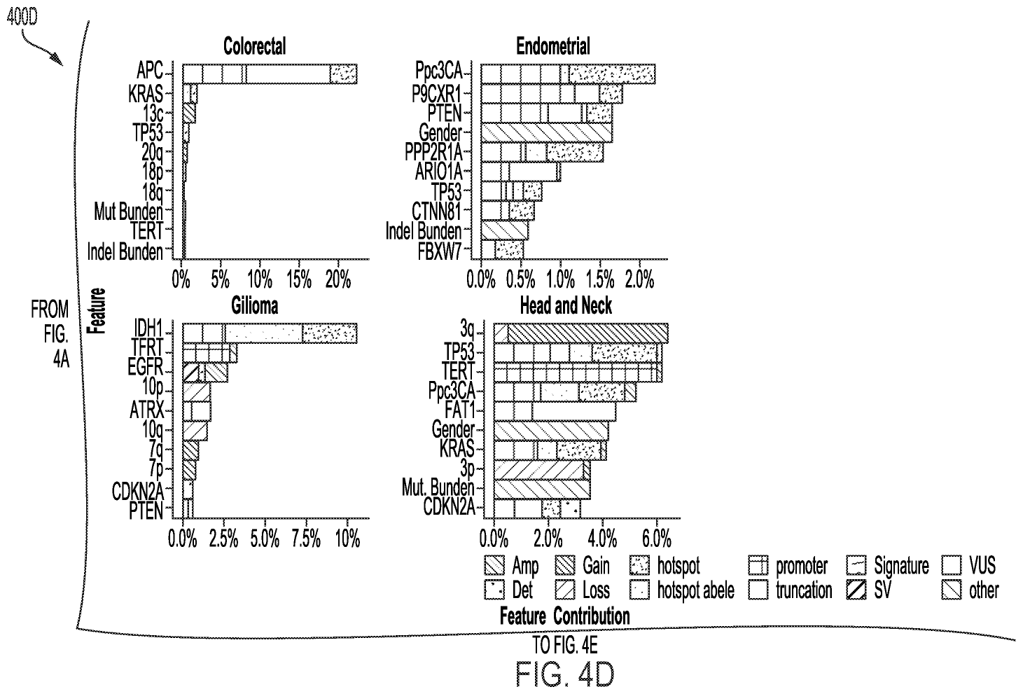

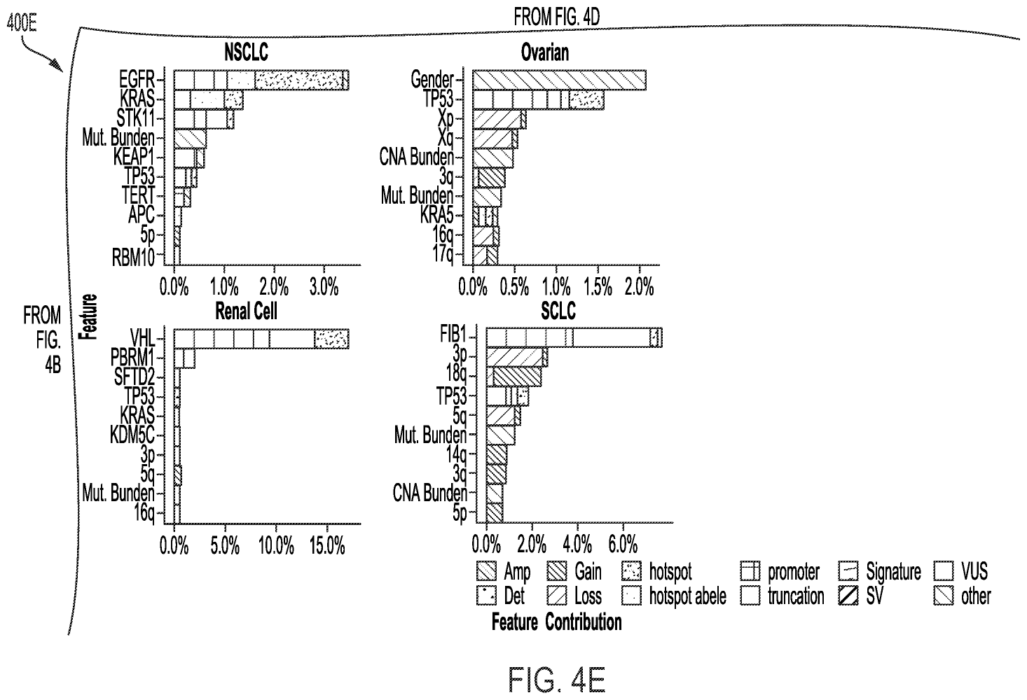

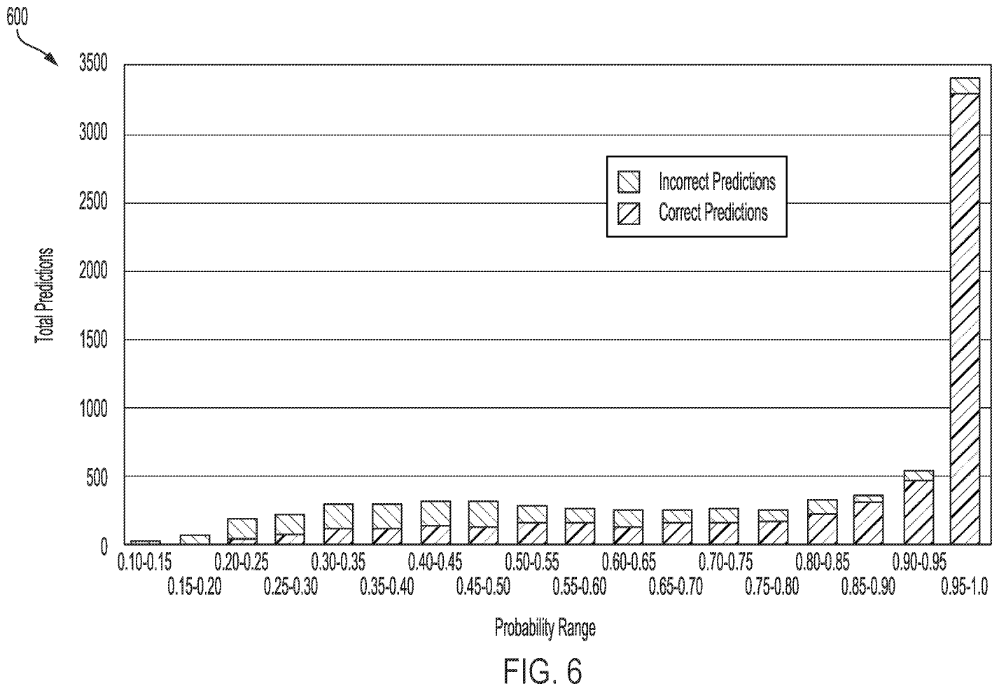

Due to the importance of high-confidence predictions for clinical decision-making in individual patients, the probability associated with each individual tumor type prediction is estimated. Raw classifier scores were calibrated to match empirically observed classification probabilities from cross-validation (log loss 0.98, FIG. 3A). In many cancer types, approximately half or more cases were classified with >95% probability (FIG. 1C). In other challenging cancer types such as esophagogastric, ovarian, and head and neck cancer, only a minority of cases were predicted with confidence>50% owing to increased molecular heterogeneity among tumors and the lack of distinguishing genomic alterations. Nevertheless, 43% of all cases were predicted with probability>95% and an empirical accuracy of 96.6%, indicating an abundance of high-confidence, reliable predictions enabled by the classifier (FIG. 6). Moreover, the majority of all incorrect predictions were made with low confidence (probability<50%) and are therefore unlikely to influence tumor identification or clinical decisions.

### Relative Predictive Value of Molecular Features

Given the diverse categories of genomic features incorporated into the classifier (Table 5), the relative importance of each molecular alteration type to the overall classification performance may be determined. Using the Cohen's kappa metric to represent overall accuracy, it was found that somatic substitutions and indels had the highest predictive value, followed by chromosome arm-level (broad) copy number alterations (CNAs) (FIG. 3A). Broad CNAs were especially informative for predicting tumor types with a low mutational burden and few other distinguishing features, such as prostate cancers lacking TMPRSS2-ERG fusions, neuroblastomas, germ cell tumors, and certain gastrointestinal cancers. Moreover, different feature categories contributed to prediction accuracy to differing degrees for individual cancer types, reinforcing the value of diverse feature categories for broad applicability and prediction accuracy (FIG. 3B).

Likewise, there was great breadth and variability among the specific features utilized to predict different cancer types (FIG. 3C, FIGS. 1A-1C). Among all individual features, truncating APC mutation was the most informative overall due to its high prevalence in, and specificity for, colorectal cancer. TERT promoter mutations occurred at high frequency in multiple tumor types, but in others they were entirely absent, leading to strongly positive and negative associations for different lineages. In other instances, more subtle patterns were evident, such as the position of mutant alleles within genes as for EGFR-mutant lung cancers and gliomas. The absence of common features also contributed to predictions of certain tumor types, such as KRAS mutations and breast cancer (FIG. 3C). In summary, these results reveal the diversity of individual genomic features and feature categories that drive tumor type predictions.

Next, it may be sought to determine whether such feature diversity and feature interaction could discriminate among different tumor types that nevertheless share a common molecular feature that is therefore not discriminatory. In BRAF V600E-mutant melanomas, colorectal, and thyroid cancers, where response rates to RAF inhibitor therapies vary, the classifier correctly predicted the tissue of origin in 162/195 cases (83%). Despite the presence of BRAF V600E in all cases, high confidence predictions were driven by distinct co-occurring mutations and genomic features, such as TERT promoter mutations in melanoma and thyroid cancer, APC mutations and microsatellite instability in colorectal cancer, and UV-associated signatures in melanoma (FIG. 3D). Misclassifications were largely due to either low tumor purity or rare atypical genomic profiles (e.g., melanomas with APC truncating mutations). These results highlight the power of incorporating multiple diverse categories of molecular aberrations to drive challenging cancer type classifications when they share individual alterations in common in various potential embodiments.

### Application to Cell Free DNA

Various embodiments of the disclosed approach may employ training data from tissue biopsies of solid tumors. Using non-invasive molecular profiling of plasma circulating tumor DNA (ctDNA), a suggested classification of patients receiving cancer screening or with inaccessible disease may be inferred in various embodiments of the disclosure. The predictive power of an embodiment of the classifier may be tested in two independent cohorts: 19 patients with genitourinary cancers and MSK-IMPACT sequencing of ctDNA, and a set of 41 patients with metastatic breast or prostate cancer and whole exome sequencing (WES) of ctDNA. Corrected predicted was the tumor type from MSK-IMPACT in 12/19 (63%) patients with prostate, bladder, and testicular cancer from among the 22 cancer types included in the classifier, including 8/8 predictions with probability>85%. Only 1 prediction (out of 10) with probability>75% was inaccurate; a prostate cancer with a single missense mutation in VHL was incorrectly predicted as renal cell carcinoma. Also, the tumor type from WES in 23/27 (85%) patients with breast cancer and in 10/14 (71%) patients with prostate cancer was correctly predicted, demonstrating the general applicability of the classifier to multiple sequencing platforms as well as its suitability for diverse specimen types such as ctDNA.

### Application of Various Embodiments to Challenging Clinical Scenarios

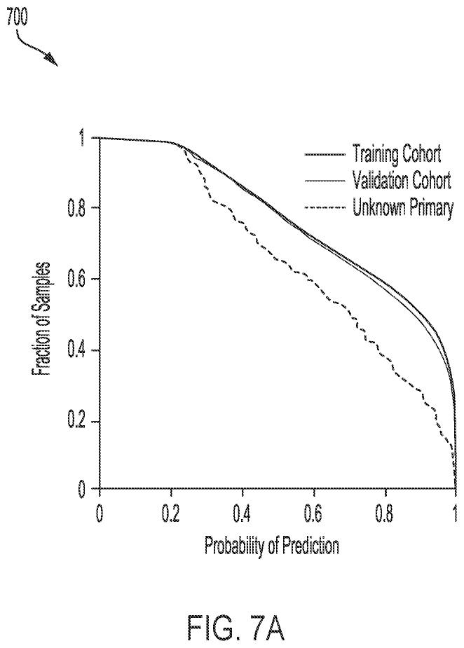

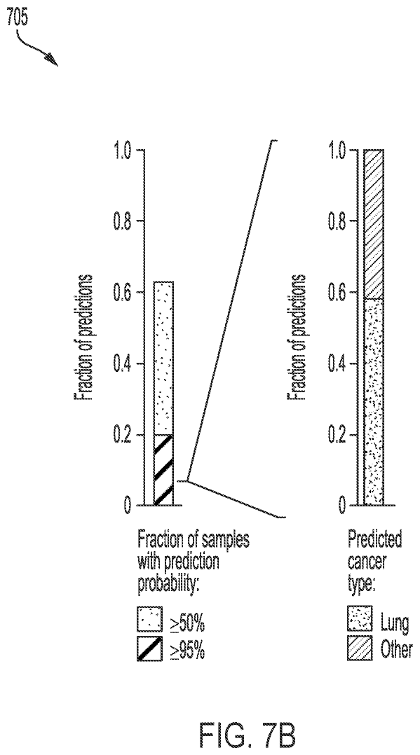

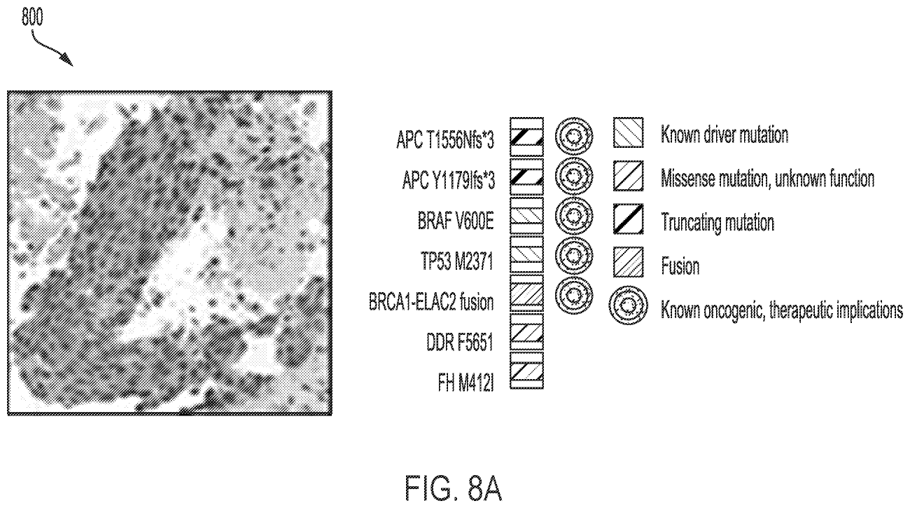

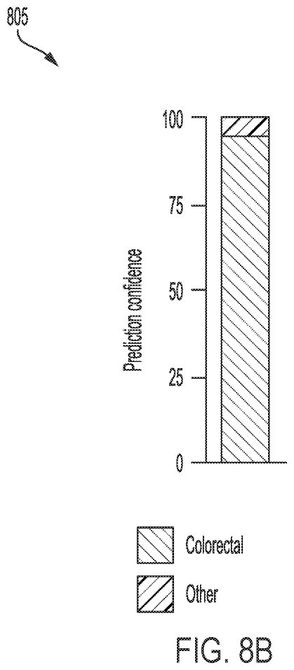

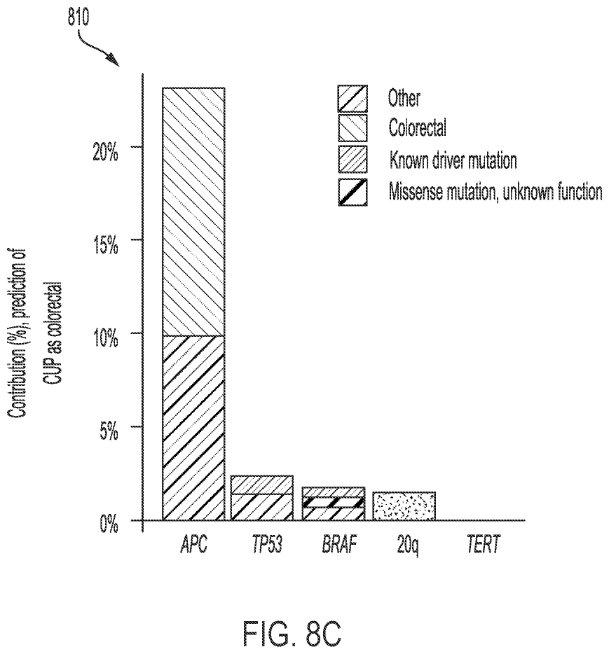

Given the predictive power of embodiments of the disclosed classifier, it was sought to determine the impact of real-time molecularly-driven classifications in multiple challenging clinical scenarios. One unmet clinical need for such accurate classification is the inference of the tissue of origin for cancers of unknown primary site (CUP). Refining tumor classification in this population can facilitate selection of potentially effective routine and investigational therapies. Using an embodiment of a trained predictive model, a likely tissue of origin may be predicted with, for example, a probability>50% in 67% (95/141) of patients (FIGS. 7A and 7B). While histopathological assessment was unable to produce a definitive classification for these patients, molecularly-driven classifications frequently supported clinical suspicions; for instance, of 29 patients with predicted non-small cell lung cancer (>50%), 28/29 had a self-reported history of smoking. In a separate example, emphasizing the need for tissue of origin classification even in an era of molecularly targeted therapy, a colorectal origin may be predicted for one CUP with 96% probability based largely on the presence of BRAF V600E and biallelic inactivating APC mutations (FIGS. 8A-8C). As single agent RAF inhibition has little activity in colon cancer, the inferred classification suggested that combined BRAF, MEK, and EGFR therapy may be required to elicit a response.

In various embodiments, the classifier of the predictive model could help resolve the uncertainty that arises in distinguishing between primary brain tumors and metastatic tumors to the central nervous system (CNS). Including both cohorts, 299 brain metastases of solid tumors originating outside the CNS may be sequenced, including 133 non-small cell lung cancers, 56 breast cancers, 43 melanomas, and 67 other tumors. The correct tumor type in 83% (248/299) of cases was correctly predicted. Importantly, out of 51 incorrect predictions, only 2 were predicted as glioma. These results illustrate the predictive value of the classifier for CNS tumors and its promise for non-invasive ctDNA profiling from cerebrospinal fluid.

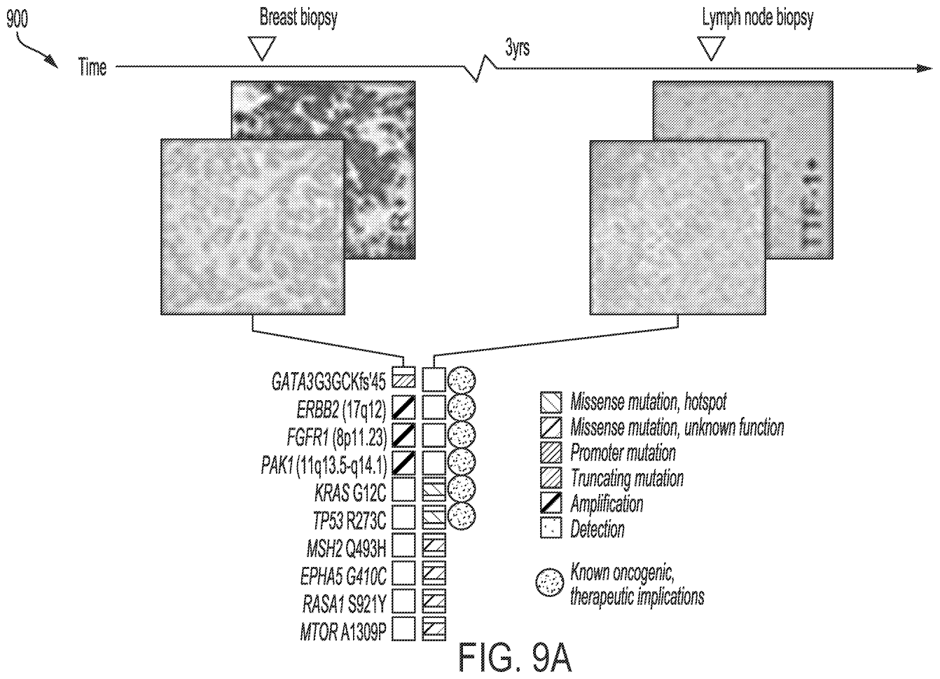

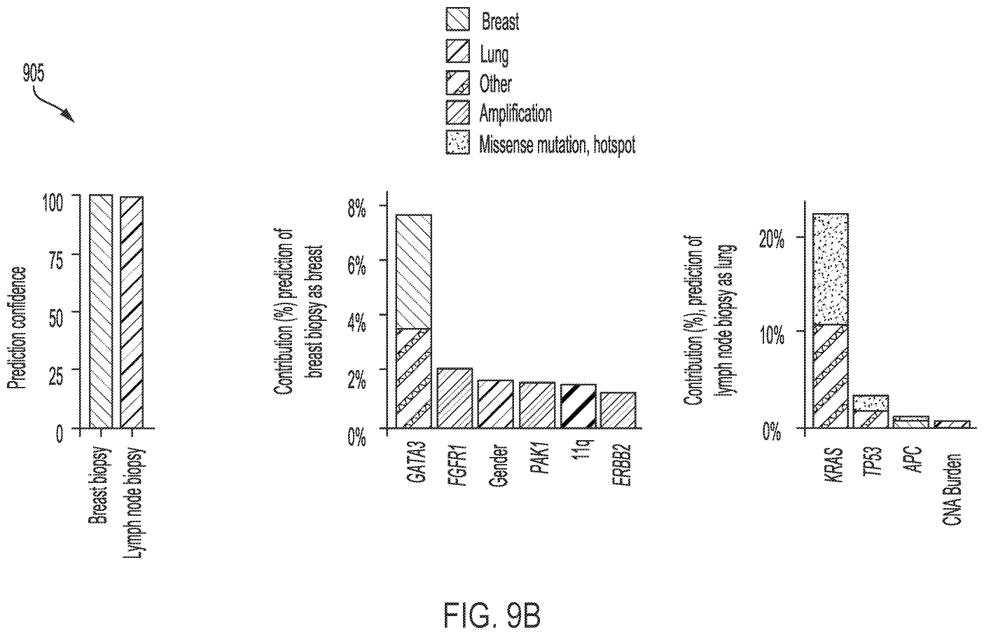

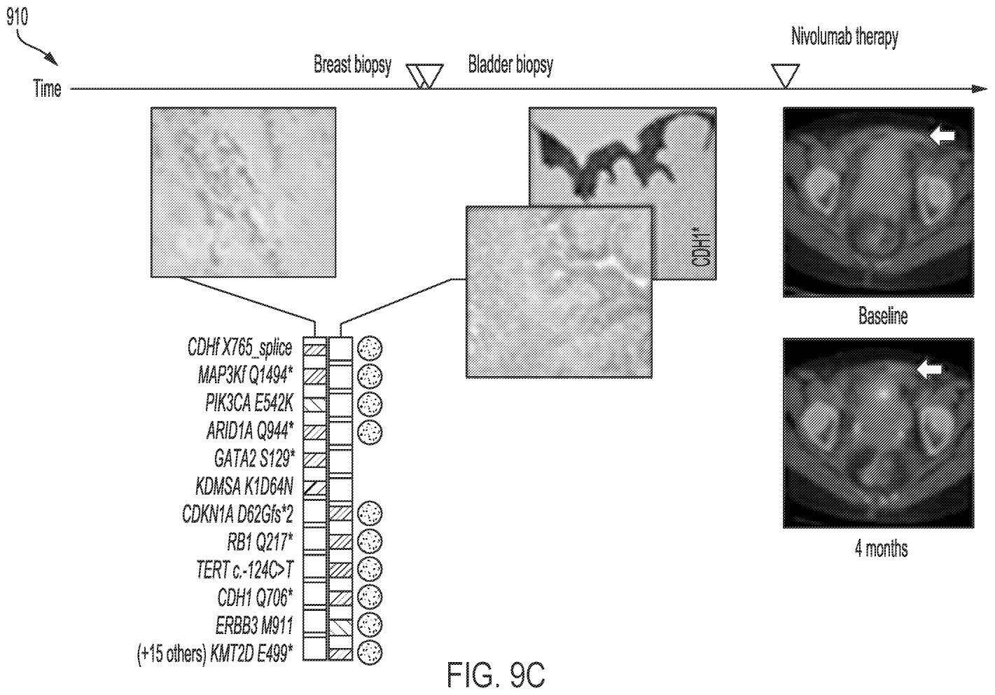

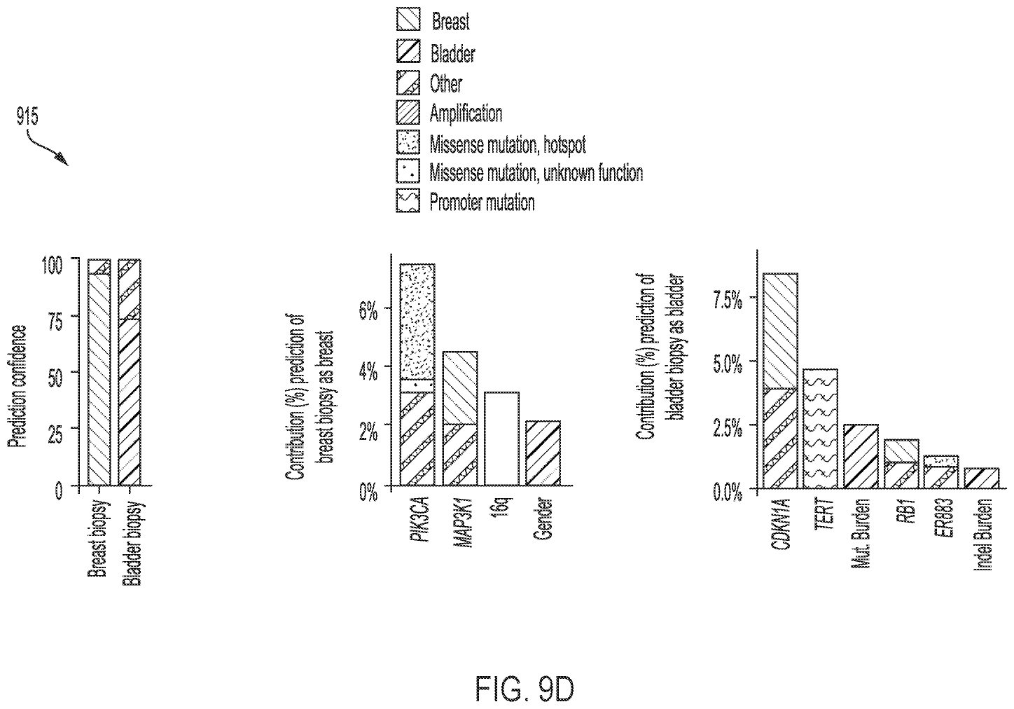

Another common and complex challenge occurs when patients with a history of cancer present with a new tumor that may represent either a distant metastasis of their prior tumor classification or a second primary tumor. Therefore, various embodiments may employ molecularly driven classifications to clarify such complex distinctions between tumor types. In one representative case, a 67-year old female with a history of breast cancer presented with a lymph node lesion three years after her initial classification. Histopathological assessment suggested metastatic poorly differentiated adenocarcinoma with micropapillary and apocrine cytology, and immunohistochemistry showed weak-to-moderate estrogen receptor staining, collectively leading to a classification of estrogen receptor-positive (ER+) breast cancer and a planned regimen of hormonal therapy (FIGS. 9A and 9B). However, concurrent clinical sequencing revealed a high mutational burden including KRAS G12C and other mutations, producing a high-confidence classification of non-small cell lung cancer (99%). These computational findings, acquired in real time, prompted additional lung cancer-specific immunohistochemistry, leading to a revised classification of metastatic lung adenocarcinoma. To reaffirm the patient's initial classification, the original primary breast tumor was subsequently obtained and sequenced and no shared mutations, a somatic GATA3 truncating mutation, and a predicted classification of breast cancer (99%) were identified. The resulting change of classification to metastatic lung cancer prompted a change in the treatment plan from hormonal therapy to chemotherapy for this patient.

Two cancers in a single patient may occasionally share mechanisms of pathogenesis that further complicate the distinction between metastatic progression and independent primary tumors. In a representative case, a 77-year-old female was referred to the center with lesions in the breast and bladder and a classification of metastatic breast lobular carcinoma (FIGS. 9A and 9B). Clinical sequencing of the bladder lesion revealed 22 somatic mutations including in the TERT promoter, CDH1, and RBI, and an APOBEC-associated mutational signature, producing a prediction of bladder cancer (74%). This prediction prompted subsequent histopathological analysis that confirmed a classification of plasmacytoid bladder cancer with corresponding loss of E-Cadherin. Indeed, CDH1 loss-of-function mutations, while not generally predictive of bladder cancer (occurring more often in lobular breast and diffuse gastric cancers), are the defining feature of plasmacytoid bladder tumors. Sequencing may be performed on the breast biopsy, which revealed 10 independent somatic mutations including a different CDH1 mutation (X765_splice), which together were predictive of breast cancer (92%). The realization that the bladder lesion was a synchronous primary tumor rather than a clonally-related metastasis led to consideration of surgical intervention as well as genetic testing for a cancer-predisposing germline mutation in CDH1. The classification of bladder cancer also ultimately facilitated on-label treatment with the immune checkpoint inhibitor nivolumab, to which the patient responded. Taken together, these representative clinical cases illustrate how genome-directed classification provides orthogonal classification resolution that, when integrated with pathology, can lead to different therapeutic modalities including surgery, hormonal therapy, chemotherapy, immunotherapy, and targeted therapy.

In various embodiments, a systematic computational approach may be developed and deployed for molecularly-driven prediction of the site of origin of tumors based on targeted DNA sequencing. While tumor sequencing is rapidly being adopted as a routine test in clinical cancer care, its impact thus far has been largely limited to driving new enrollments onto clinical trials and for the identification of biomarkers of treatment response and resistance. In various embodiments, such sequencing informs cancer classification, potentially as an adjunct to histopathologic assessment. In this approach, multi-faceted molecular alteration types may be incorporated into a probabilistic prediction to accurately identify therapeutically significant cancer type differences under challenging classification circumstances.

Various embodiments may have a wide array of clinical applications. Genome-directed classification, as typified by the representative cases here, can alter patient eligibility for various clinical modalities. As liquid biopsy is increasingly used as a screening tool for cancer recurrence and new malignancies, the approach can inform the site of origin when ctDNA is detected. There are also many ways in which predictions may be utilized clinically, especially in light of the development of probability estimates on individual predictions. In cases in which traditional classification is ambiguous or challenging, computational predictions from genomic data can exclude possibilities even if the predictions are not definitive. In other cases, a high-confidence prediction that disagrees with the defined or suspected classification can prompt pathological and clinical re-evaluation, allowing additional testing that may help support an alternative classification. In contrast to using mRNA-based tissue classification to predict the site of origin for CUP, an advantage of embodiments of the disclosed approach is their ability to enumerate the discrete genomic features driving individual predictions, thereby providing pathologists and oncologists an opportunity to rationally interpret discordant results.

The high accuracy of the classifier, trained on MSK-IMPACT data, for predicting tumor type from ctDNA WES data suggests broad applicability to other panels with shared genomic targets. The disclosed approach may resolve challenging classification scenarios, alter established classifications (via prompting of additional pathological assessment), and affect therapeutic modalities.

Overall, as the understanding improves of how lineage influences response to the newest generation of therapies in cancer, embodiments of the disclosed systematic approach to molecularly-driven classification coupled to clinical histories, histopathologic assessment, and imaging will improve classifications and treatment decisions. The results exemplify the emerging and powerful role of artificial intelligence in medicine for clinical decision support.

### Supplementary Content for Various Potential Embodiments

### Detailed Methods

### Training Set

The dataset was derived from the MSK-IMPACT (Memorial Sloan Kettering-Integrated Mutation Profiling of Actionable Cancer Targets) clinical series and includes samples from cancer patients among more than 60 cancer types. Patients predominantly exhibited advanced metastatic disease, and all patients consented to somatic mutation profiling in a CLIA-compliant laboratory. The cancer type and primary site classifications for each sample in this cohort were determined and recorded in real time as part of the clinical workup of each case. Molecular pathology fellows reviewed the surgical pathology report available at the time of MSK-IMPACT testing and selected the most appropriate OncoTree code representing the detailed tumor type. In total, 22 major cancer types with more than 40 independent tumors were selected for this analysis (Table 1). Samples that were not associated with a classification of one of these 22 selected cancer types were excluded from the training set.

TABLE 1
Distinct tumor types considered for classification

| CANCER_TYPE | CANCER_TYPE_DETAILED |
|---|---|
| Bladder.Cancer | Bladder Urothelial Carcinoma \| Upper Tract Urothelial Carcinoma |
| Breast.Cancer | Adenoid Cystic Breast Cancer \| Breast Carcinoma \| Breast Invasive |
| | Cancer, NOS \| Breast Invasive Carcinoma, NOS \| Breast Invasive |
| | Ductal Carcinoma \| Breast Invasive Lobular Carcinoma \| Breast |
| | Invasive Mixed Mucinous Carcinoma \| Breast Mixed Ductal and |
| | Lobular Carcinoma \| Metaplastic Breast Cancer |
| Cholangiocarcinoma | Cholangiocarcinoma \| Extrahepatic Cholangiocarcinoma \| |
| | Intrahepatic Cholangiocarcinoma \| Perihilar Cholangiocarcinoma |
| Colorectal.Cancer | Colon Adenocarcinoma \| Colorectal Adenocarcinoma \| Medullary |
| | Carcinoma of the Colon \| Mucinous Adenocarcinoma of the Colon |
| | and Rectum \| Mucinous Colorectal Carcinoma \| Rectal |
| | Adenocarcinoma |
| Endometrial.Cancer | Endometrial Carcinoma \| Uterine Carcinosarcoma/Uterine |
| | Malignant Mixed Mullerian Tumor \| Uterine Clear Cell Carcinoma \| |
| | Uterine Dedifferentiated Carcinoma \| Uterine Endometrioid |
| | Carcinoma \| Uterine Mixed Endometrial Carcinoma \| Uterine |
| | Neuroendocrine Carcinoma \| Uterine Serous Carcinoma/Uterine |
| | Papillary Serous Carcinoma \| Uterine Undifferentiated Carcinoma |
| Esophagogastric.Cancer | Adenocarcinoma of the Gastroesophageal Junction \| Esophageal |
| | Adenocarcinoma \| Esophageal Squamous Cell Carcinoma \| |
| | Esophagogastric Adenocarcinoma \| Intestinal Type Stomach |
| | Adenocarcinoma \| Poorly Differentiated Carcinoma of the Stomach \| |
| | Signet Ring Cell Carcinoma of the Stomach \| Stomach |
| | Adenocarcinoma \| Tubular Stomach Adenocarcinoma |
| Gastrointestinal.Stromal.Tumor | Gastrointestinal Stromal Tumor |
| Germ.Cell.Tumor | Embryonal Carcinoma \| Immature Teratoma \| Mature Teratoma \| |
| | Mixed Germ Cell Tumor \| Non-Seminomatous Germ Cell Tumor \| |
| | Seminoma \| Teratoma \| Teratoma with Malignant Transformation \| |
| | Yolk Sac Tumor |
| Glioma | Anaplastic Astrocytoma \| Anaplastic Ganglioglioma \| Anaplastic |
| | Oligoastrocytoma \| Anaplastic Oligodendroglioma \| Astrocytoma \| |
| | Diffuse Intrinsic Pontine Glioma \| Ganglioglioma \| Glioblastoma |
| | Multiforme \| Gliosarcoma \| High-Grade Glioma, NOS \| Low-Grade |
| | Glioma, NOS \| Oligoastrocytoma \| Oligodendroglioma \| Pilocytic |
| | Astrocytoma \| Pleomorphic Xanthoastrocytoma |
| Head.and.Neck.Cancer | Clear Cell Odontogenic Carcinoma \| Epithelial-Myoepithelial |
| | Carcinoma \| Head and Neck Carcinoma, Other \| Head and Neck |
| | Neuroendocrine Carcinoma \| Head and Neck Squamous Cell |
| | Carcinoma \| Head and Neck Squamous Cell Carcinoma of Unknown |
| | Primary \| Hypopharynx Squamous Cell Carcinoma \| Larynx |
| | Squamous Cell Carcinoma \| Nasopharyngeal Carcinoma \| |
| | Odontogenic Carcinoma \| Oral Cavity Squamous Cell Carcinoma \| |
| | Oropharynx Squamous Cell Carcinoma \| Sinonasal Adenocarcinoma |
| | \| Sinonasal Squamous Cell Carcinoma \| Sinonasal Undifferentiated |
| | Carcinoma |
| Melanoma | Acral Melanoma \| Anorectal Mucosal Melanoma \| Cutaneous |
| | Melanoma \| Desmoplastic Melanoma \| Genitourinary Mucosal |
| | Melanoma \| Head and Neck Mucosal Melanoma \| Melanoma of |
| | Unknown Primary \| Mucosal Melanoma of the Esophagus \| Mucosal |
| | Melanoma of the Urethra \| Mucosal Melanoma of the Vulva/Vagina |
| | \| Primary CNS Melanoma |
| Mesothelioma | Peritoneal Mesothelioma \| Pleural Mesothelioma \| Pleural |
| | Mesothelioma, Biphasic Type \| Pleural Mesothelioma, Epithelioid |
| | Type \| Pleural Mesothelioma, Sarcomatoid Type \| Testicular |
| | Mesothelioma |
| Neuroblastoma | Neuroblastoma |
| Non.Small.Cell.Lung.Cancer | Atypical Lung Carcinoid \| Basaloid Large Cell Carcinoma of the |
| | Lung \| Ciliated Muconodular Papillary Tumor of the Lung \| Large |
| | Cell Lung Carcinoma \| Large Cell Neuroendocrine Carcinoma \| |
| | Lung Adenocarcinoma \| Lung Adenosquamous Carcinoma \| Lung |
| | Carcinoid \| Lung Squamous Cell Carcinoma \| Lymphoepithelioma- |
| | like Carcinoma of the Lung \| Non-Small Cell Lung Cancer \| |
| | Pleomorphic Carcinoma of the Lung \| Poorly Differentiated Non- |
| | Small Cell Lung Cancer \| Sarcomatoid Carcinoma of the Lung \| |
| | Spindle Cell Carcinoma of the Lung |
| Ovarian.Cancer | Clear Cell Ovarian Cancer \| Endometrioid Ovarian Cancer \| High- |
| | Grade Neuroendocrine Carcinoma of the Ovary \| High-Grade Serous |
| | Ovarian Cancer \| Low-Grade Serous Ovarian Cancer \| Mixed |
| | Ovarian Carcinoma \| Mucinous Ovarian Cancer \| Ovarian Cancer, |
| | Other \| Ovarian Carcinosarcoma/Malignant Mixed Mesodermal |
| | Tumor \| Ovarian Epithelial Tumor \| Ovarian Seromucinous |
| | Carcinoma \| Serous Borderline Ovarian Tumor \| Serous Borderline |
| | Ovarian Tumor, Micropapillary \| Serous Ovarian Cancer \| Small |
| | Cell Carcinoma of the Ovary |
| Pancreatic.Cancer | Acinar Cell Carcinoma of the Pancreas \| Adenosquamous |
| | Carcinoma of the Pancreas \| Intraductal Papillary Mucinous |
| | Neoplasm \| Mucinous Cystic Neoplasm \| Pancreatic |
| | Adenocarcinoma \| Pancreatoblastoma \| Serous Cystadenoma of the |
| | Pancreas \| Solid Pseudopapillary Neoplasm of the Pancreas \| |
| | Undifferentiated Carcinoma of the Pancreas |
| Pancreatic.Neuroendocrine.Tumor | Pancreatic Neuroendocrine Tumor |
| Prostate.Cancer | Prostate Adenocarcinoma \| Prostate Neuroendocrine Carcinoma \| |
| | Prostate Small Cell Carcinoma |
| Renal.Cell.Cancer | Chromophobe Renal Cell Carcinoma \| Collecting Duct Renal Cell |
| | Carcinoma \| Papillary Renal Cell Carcinoma \| Renal |
| | Angiomyolipoma \| Renal Cell Carcinoma \| Renal Clear Cell |
| | Carcinoma \| Renal Clear Cell Carcinoma with Sarcomatoid Features |
| | \| Renal Medullary Carcinoma \| Renal Mucinous Tubular Spindle |
| | Cell Carcinoma \| Renal Oncocytoma \| Translocation-Associated |
| | Renal Cell Carcinoma \| Unclassified Renal Cell Carcinoma |
| Small.Cell.Lung.Cancer | Lung Neuroendocrine Tumor \| Small Cell Lung Cancer |
| Thyroid.Cancer | Anaplastic Thyroid Cancer \| Follicular Thyroid Cancer \| Hurthle |
| | Cell Thyroid Cancer \| Medullary Thyroid Cancer \| Papillary Thyroid |
| | Cancer \| Poorly Differentiated Thyroid Cancer |
| Uveal.Melanoma | Uveal Melanoma |
| Total | |

The MSK-IMPACT cohort includes many samples derived from biopsy specimens with often low tumor content. Such samples can have reduced sensitivity for detection for genomic alterations, especially changes in DNA copy number. In order to reduce associated bias in the frequency of the genomic alterations defining each cancer type, samples for which all mutations have a somatic mutant allele frequency less than 1000 and with copy number alterations with an absolute log ratio less than 0.2 were excluded from the training set. Samples with no evident genomic alterations were also excluded from the training set and were not used for prediction. Only one sample per patient was included, with preference given to primary over metastatic samples. In total, the training set excluded samples from less frequent cancer types, samples from low purity specimens, and redundant samples from patients with more than one tumor specimen sequenced. The resulting training cohort included samples. Prediction accuracy may be determined for samples in the training set using five-fold cross-validation. An independent set of tumors subsequently profiled using MSK-IMPACT as part of the same prospective clinical sequencing initiative was used to test the accuracy of the classifier. Demographic characteristics of both cohorts are displayed in Table 2.

TABLE 2
Clinical and technical characteristics
of the training and validation cohorts

| | | TRAINING | VALIDATION |
|---|---|---|---|
| | | COHORT | COHORT |
| Age at Sequencing | mean | 60.3 | 62.1 |
| | median | 62 | 64 |
| | SD | 14.5 | 13.7 |
| Tumor Purity | mean | 45.5 | 39.1 |
| | median | 40 | 40 |
| | SD | 21.3 | 20.4 |
| Sequence Coverage | mean | 718 | 676 |
| | SD | 268 | 199 |
| Mutations | mean | 8 | 8.8 |
| | median | 5 | 4 |
| | SD | 18.1 | 22.4 |
| Fraction Genome | mean | 0.21 | 0.19 |
| Altered | median | 0.17 | 0.13 |
| | SD | 0.19 | 0.19 |

TABLE 3
Sensitivity and specificity of predictions for each tumor type

| | Total | Accurate | | |
|---|---|---|---|---|
| Cancer Type | Predictions | Predictions | Sensitivity | Specificity |
| Non.Small.Cell.Lung.Cancer | 1600 | 1099 | 0.782 | 0.687 |
| Breast.Cancer | 1360 | 1035 | 0.876 | 0.761 |
| Colorectal.Cancer | 892 | 785 | 0.847 | 0.880 |
| Prostate.Cancer | 550 | 423 | 0.812 | 0.769 |
| Glioma | 500 | 440 | 0.873 | 0.880 |
| Bladder.Cancer | 342 | 274 | 0.765 | 0.801 |
| Pancreatic.Cancer | 372 | 248 | 0.719 | 0.667 |
| Renal.Cell.Cancer | 293 | 217 | 0.707 | 0.741 |
| Melanoma | 267 | 205 | 0.707 | 0.768 |
| Esophagogastric.Cancer | 246 | 119 | 0.431 | 0.484 |
| Germ.Cell.Tumor | 243 | 191 | 0.799 | 0.786 |
| Thyroid.Cancer | 189 | 113 | 0.523 | 0.598 |
| Ovarian.Cancer | 160 | 73 | 0.348 | 0.456 |
| Endometrial.Cancer | 146 | 99 | 0.495 | 0.678 |
| Cholangiocarcinoma | 117 | 63 | 0.364 | 0.538 |
| Head.and.Neck.Cancer | 91 | 55 | 0.320 | 0.604 |
| Gastrointestinal.Stromal.Tumor | 118 | 88 | 0.727 | 0.746 |
| Mesothelioma | 85 | 51 | 0.537 | 0.600 |
| Small.Cell.Lung.Cancer | 62 | 48 | 0.552 | 0.774 |
| Pancreatic.Neuroendocrine.Tumor | 64 | 41 | 0.621 | 0.641 |
| Neuroblastoma | 50 | 42 | 0.737 | 0.840 |
| Uveal.Melanoma | 44 | 39 | 0.951 | 0.886 |

TABLE 4
Prediction accuracy for detailed histological subtypes

| | | Accurate | |
|---|---|---|---|
| Cancer Type | Cancer Type Detailed | Predictions | Sensitivity |
| Bladder.Cancer | Bladder Urothelial Carcinoma | 223 | 0.78 |
| Bladder.Cancer | Upper Tract Urothelial Carcinoma | 51 | 0.70 |
| Breast.Cancer | Breast Invasive Ductal Carcinoma | 767 | 0.87 |
| Breast.Cancer | Breast Invasive Lobular | 167 | 0.95 |
| | Carcinoma | | |
| Breast.Cancer | Breast Mixed Ductal and Lobular | 46 | 0.88 |
| | Carcinoma | | |
| Breast.Cancer | Breast Invasive Carcinoma, NOS | 23 | 0.70 |
| Breast.Cancer | Breast Invasive Cancer, NOS | 17 | 0.94 |
| Breast.Cancer | Other | 15 | 0.83 |
| Cholangiocarcinoma | Intrahepatic Cholangiocarcinoma | 46 | 0.46 |
| Cholangiocarcinoma | Cholangiocarcinoma, NOS | 14 | 0.28 |
| Cholangiocarcinoma | Extrahepatic Cholangiocarcinoma | 3 | 0.14 |
| Cholangiocarcinoma | Other | 0 | 0.00 |
| Colorectal.Cancer | Colon Adenocarcinoma | 555 | 0.85 |
| Colorectal.Cancer | Rectal Adenocarcinoma | 192 | 0.89 |
| Colorectal.Cancer | Mucinous Adenocarcinoma of the | 24 | 0.69 |
| | Colon and Rectum | | |
| Colorectal.Cancer | Colorectal Adenocarcinoma | 12 | 0.75 |
| Colorectal.Cancer | Other | 2 | 0.67 |
| Endometrial.Cancer | Uterine Endometrioid Carcinoma | 58 | 0.67 |
| Endometrial.Cancer | Uterine Serous Carcinoma/Uterine | 20 | 0.45 |
| | Papillary Serous Carcinoma | | |
| Endometrial.Cancer | Uterine Carcinosarcoma/Uterine | 9 | 0.26 |
| | Malignant Mixed Mullerian | | |
| | Tumor | | |
| Endometrial.Cancer | Uterine Mixed Endometrial | 6 | 0.35 |
| | Carcinoma | | |
| Endometrial.Cancer | Uterine Clear Cell Carcinoma | 3 | 0.21 |
| Endometrial.Cancer | Other | 3 | 0.60 |
| Esophagogastric.Cancer | Stomach Adenocarcinoma | 42 | 0.34 |
| Esophagogastric.Cancer | Esophageal Adenocarcinoma | 55 | 0.54 |
| Esophagogastric.Cancer | Adenocarcinoma of the | 20 | 0.54 |
| | Gastroesophageal Junction | | |
| Esophagogastric.Cancer | Esophageal Squamous Cell | 1 | 0.11 |
| | Carcinoma | | |
| Esophagogastric.Cancer | Other | 1 | 0.17 |
| Gastrointestinal.Stromal.Tumor | Gastrointestinal Stromal Tumor | 88 | 0.73 |
| Germ.Cell.Tumor | Mixed Germ Cell Tumor | 95 | 0.87 |
| Germ.Cell.Tumor | Seminoma | 54 | 0.81 |
| Germ.Cell.Tumor | Yolk Sac Tumor | 8 | 0.38 |
| Germ.Cell.Tumor | Non-Seminomatous Germ Cell | 14 | 0.78 |
| | Tumor | | |
| Germ.Cell.Tumor | Embryonal Carcinoma | 15 | 0.94 |
| Germ.Cell.Tumor | Other | 5 | 0.63 |
| Glioma | Glioblastoma Multiforme | 237 | 0.89 |
| Glioma | Anaplastic Astrocytoma | 65 | 0.86 |
| Glioma | Anaplastic Oligodendroglioma | 39 | 0.98 |
| Glioma | Oligodendroglioma | 34 | 0.94 |
| Glioma | Astrocytoma | 27 | 0.84 |
| Glioma | Anaplastic Oligoastrocytoma | 13 | 0.93 |
| Glioma | High-Grade Glioma, NOS | 7 | 0.50 |
| Glioma | Other | 18 | 0.69 |
| Head.and.Neck.Cancer | Head and Neck Squamous Cell | 13 | 0.31 |
| | Carcinoma | | |
| Head.and.Neck.Cancer | Oral Cavity Squamous Cell | 21 | 0.55 |
| | Carcinoma | | |
| Head.and.Neck.Cancer | Oropharynx Squamous Cell | 12 | 0.32 |
| | Carcinoma | | |
| Head.and.Neck.Cancer | Larynx Squamous Cell Carcinoma | 1 | 0.08 |
| Head.and.Neck.Cancer | Nasopharyngeal Carcinoma | 3 | 0.25 |
| Head.and.Neck.Cancer | Head and Neck Squamous Cell | 5 | 0.17 |
| | Carcinoma of Unknown Primary | | |
| Melanoma | Cutaneous Melanoma | 139 | 0.79 |
| Melanoma | Melanoma of Unknown Primary | 36 | 0.90 |
| Melanoma | Acral Melanoma | 8 | 0.38 |
| Melanoma | Anorectal Mucosal Melanoma | 12 | 0.60 |
| Melanoma | Mucosal Melanoma of the | 4 | 0.27 |
| | Vulva/Vagina | | |
| Melanoma | Head and Neck Mucosal | 4 | 0.36 |
| | Melanoma | | |
| Melanoma | Other | 2 | 0.29 |
| Mesothelioma | Pleural Mesothelioma, Epithelioid | 20 | 0.53 |
| | Type | | |
| Mesothelioma | Pleural Mesothelioma | 22 | 0.67 |
| Mesothelioma | Peritoneal Mesothelioma | 6 | 0.35 |
| Mesothelioma | Other | 3 | 0.43 |
| Neuroblastoma | Neuroblastoma | 42 | 0.74 |
| Non.Small.Cell.Lung.Cancer | Lung Adenocarcinoma | 923 | 0.81 |
| Non.Small.Cell.Lung.Cancer | Lung Squamous Cell Carcinoma | 100 | 0.68 |
| Non.Small.Cell.Lung.Cancer | Large Cell Neuroendocrine | 25 | 0.71 |
| | Carcinoma | | |
| Non.Small.Cell.Lung.Cancer | Poorly Differentiated Non-Small | 15 | 0.68 |
| | Cell Lung Cancer | | |
| Non.Small.Cell.Lung.Cancer | Non-Small Cell Lung Cancer | 11 | 0.79 |
| Non.Small.Cell.Lung.Cancer | Atypical Lung Carcinoid | 3 | 0.23 |
| Non.Small.Cell.Lung.Cancer | Sarcomatoid Carcinoma of the | 7 | 0.54 |
| | Lung | | |
| Non.Small.Cell.Lung.Cancer | Lung Adenosquamous Carcinoma | 7 | 0.78 |
| Non.Small.Cell.Lung.Cancer | Lung Carcinoid | 1 | 0.13 |
| Non.Small.Cell.Lung.Cancer | Other | 7 | 1.00 |
| Ovarian.Cancer | High-Grade Serous Ovarian | 59 | 0.47 |
| | Cancer | | |
| Ovarian.Cancer | Clear Cell Ovarian Cancer | 2 | 0.09 |
| Ovarian.Cancer | Low-Grade Serous Ovarian | 2 | 0.10 |
| | Cancer | | |
| Ovarian.Cancer | Ovarian | 7 | 0.64 |
| | Carcinosarcoma/Malignant Mixed | | |
| | Mesodermal Tumor | | |
| Ovarian.Cancer | Mucinous Ovarian Cancer | 0 | 0.00 |
| Ovarian.Cancer | Endometrioid Ovarian Cancer | 0 | 0.00 |
| Ovarian.Cancer | Other | 3 | 0.20 |
| Pancreatic.Cancer | Pancreatic Adenocarcinoma | 238 | 0.77 |
| Pancreatic.Cancer | Acinar Cell Carcinoma of the | 0 | 0.00 |
| | Pancreas | | |
| Pancreatic.Cancer | Intraductal Papillary Mucinous | 3 | 0.38 |
| | Neoplasm | | |
| Pancreatic.Cancer | Adenosquamous Carcinoma of the | 6 | 0.86 |
| | Pancreas | | |
| Pancreatic.Cancer | Other | 1 | 0.11 |
| Pancreatic.Neuroendocrine.Tumor | Pancreatic Neuroendocrine Tumor | 41 | 0.62 |
| Prostate.Cancer | Prostate Adenocarcinoma | 415 | 0.82 |
| Prostate.Cancer | Prostate Neuroendocrine | 3 | 0.38 |
| | Carcinoma | | |
| Prostate.Cancer | Other | 5 | 1.00 |
| Renal.Cell.Cancer | Renal Clear Cell Carcinoma | 167 | 0.93 |
| Renal.Cell.Cancer | Unclassified Renal Cell | 21 | 0.46 |
| | Carcinoma | | |
| Renal.Cell.Cancer | Papillary Renal Cell Carcinoma | 13 | 0.46 |
| Renal.Cell.Cancer | Chromophobe Renal Cell | 13 | 0.54 |
| | Carcinoma | | |
| Renal.Cell.Cancer | Translocation-Associated Renal | 1 | 0.11 |
| | Cell Carcinoma | | |
| Renal.Cell.Cancer | Other | 2 | 0.10 |
| Small.Cell.Lung.Cancer | Small Cell Lung Cancer | 48 | 0.59 |
| Small.Cell.Lung.Cancer | Lung Neuroendocrine Tumor | 0 | 0.00 |
| Thyroid.Cancer | Papillary Thyroid Cancer | 59 | 0.74 |
| Thyroid.Cancer | Poorly Differentiated Thyroid | 28 | 0.48 |
| | Cancer | | |
| Thyroid.Cancer | Anaplastic Thyroid Cancer | 14 | 0.44 |
| Thyroid.Cancer | Hurthle Cell Thyroid Cancer | 7 | 0.30 |
| Thyroid.Cancer | Medullary Thyroid Cancer | 0 | 0.00 |
| Thyroid.Cancer | Follicular Thyroid Cancer | 5 | 1.00 |
| Thyroid.Cancer | Other | 0 | 0.00 |
| Uveal.Melanoma | Uveal Melanoma | 39 | 0.95 |

### Derivation of Features

The molecular feature set was based on 341 oncogenes and tumor suppressor genes common to all MSK-IMPACT panel versions. This panel covers all exons of each gene including some relevant intronic regions to capture known structural variants, the TERT promoter and additional “tiling” SNPs to improve copy number calling. The features were derived from the following genomic alteration classes.

Somatic mutations. Mutations were annotated with Ensembl VEP. For each gene in the panel, the training set contained a binary feature corresponding to the presence or absence of a non-synonymous missense mutation and a binary feature corresponding to the presence or absence of a truncating mutation in the gene. The mutation status of known hotspot mutations and the status of the 30 distinct mutational signatures were also included as binary features. Mutational signatures were derived for each sample with at least ten synonymous or nonsynonymous somatic mutations and those signatures representing more than 40% of mutations were considered as present. The total number of nonsynonymous mutations per sample was included as a numeric feature.

Copy number alterations. The presence or absence of genomic gains and losses of each chromosome arm were identified from MSK-IMPACT data. Genomic coordinates for the chromosome arms in the GRCh37/hg19 human genome assembly were considered gained or lost if a majority of the arm (>50%) is affected by segment of absolute value of log-ratio of ±0.2. The presence or absence of focal amplifications and deep deletions (presumed homozygous deletions) for each of the 341 genes in the panel were also included as features. In addition, included may be a numeric feature representing the overall DNA copy number alteration burden, defined as the percentage of the autosomal genome that was affected by copy number alterations (gains or losses) inferred from the segmented log-ratio data.

Structural variants. The MSK-IMPACT panel includes several intronic regions designed to detect structural variants in genes that are commonly rearranged in cancer. Features were included for the presence or absence of selected structural variants detected by MSK-IMPACT (Table 5).

TABLE 5
Individual molecular features selected by the classifier

| Feature | Category | Feature | Category |
|---|---|---|---|
| AKT2_Amp | Amp | Del_7q | Loss |
| ALK_Amp | Amp | Del_8p | Loss |
| AMER1_Amp | Amp | Del_8q | Loss |
| AR_Amp | Amp | Del_9p | Loss |
| ASXL1_Amp | Amp | Del_9q | Loss |
| AURKA_Amp | Amp | Del_Xp | Loss |
| AXIN2_Amp | Amp | Del_Xq | Loss |
| BBC3_Amp | Amp | CN_Burden | Other |
| BCL2L1_Amp | Amp | Gender_F | Other |
| BCL6_Amp | Amp | LogINDEL_Mb | Other |
| BRCA1_Amp | Amp | LogSNV_Mb | Other |
| BRIP1_Amp | Amp | TERTp | Promoter |
| CARD11_Amp | Amp | Sig_APOBEC | Signature |
| CCND1_Amp | Amp | Sig_MMR | Signature |
| CCND2_Amp | Amp | Sig_UV | Signature |
| CCND3_Amp | Amp | EGFR_SV | SV |
| CCNE1_Amp | Amp | TMPRSS2_ERG_fusion | SV |
| CD274_Amp | Amp | TMRPSS2_ETV1_fusion | SV |
| CD79B_Amp | Amp | APC_TRUNC | Truncation |
| CDK12_Amp | Amp | ALK_TRUNC | Truncation |
| CDK4_Amp | Amp | AMER1_TRUNC | Truncation |
| CDK6_Amp | Amp | AR_TRUNC | Truncation |
| CDK8_Amp | Amp | ARID1A_TRUNC | Truncation |
| CDKN1B_Amp | Amp | ARID1B_TRUNC | Truncation |
| CRKL_Amp | Amp | ARID2_TRUNC | Truncation |
| DAXX_Amp | Amp | ASXL1_TRUNC | Truncation |
| DCUN1D1_Amp | Amp | ASXL2_TRUNC | Truncation |
| DDR2_Amp | Amp | ATM_TRUNC | Truncation |
| DIS3_Amp | Amp | ATRX_TRUNC | Truncation |
| DNMT3B_Amp | Amp | AXL_TRUNC | Truncation |
| E2F3_Amp | Amp | BAP1_TRUNC | Truncation |
| EGFR_Amp | Amp | BBC3_TRUNC | Truncation |
| ERBB2_Amp | Amp | BCOR_TRUNC | Truncation |
| ERBB3_Amp | Amp | BRCA2_TRUNC | Truncation |
| ERCC5_Amp | Amp | CARD11_TRUNC | Truncation |
| ERG_Amp | Amp | CASP8_TRUNC | Truncation |
| ETV1_Amp | Amp | CDH1_TRUNC | Truncation |
| ETV6_Amp | Amp | CDK12_TRUNC | Truncation |
| FAM46C_Amp | Amp | CDKN1A_TRUNC | Truncation |
| FGF19_Amp | Amp | CDKN2A_TRUNC | Truncation |
| FGF3_Amp | Amp | CIC_TRUNC | Truncation |
| FGF4_Amp | Amp | CREBBP_TRUNC | Truncation |
| FGFR1_Amp | Amp | CTCF_TRUNC | Truncation |
| FH_Amp | Amp | DAXX_TRUNC | Truncation |
| FLT1_Amp | Amp | EIF1AX_TRUNC | Truncation |
| FLT3_Amp | Amp | EP300_TRUNC | Truncation |
| FOXA1_Amp | Amp | EPHA3_TRUNC | Truncation |
| GNAS_Amp | Amp | FAT1_TRUNC | Truncation |
| H3F3C_Amp | Amp | FBXW7_TRUNC | Truncation |
| HIST1H1C_Amp | Amp | FLT1_TRUNC | Truncation |
| HIST1H2BD_Amp | Amp | FOXA1_TRUNC | Truncation |
| HIST1H3B_Amp | Amp | FUBP1_TRUNC | Truncation |
| IKBKE_Amp | Amp | GATA3_TRUNC | Truncation |
| IL10_Amp | Amp | GRIN2A_TRUNC | Truncation |
| IL7R_Amp | Amp | JAK1_TRUNC | Truncation |
| IRF4_Amp | Amp | KDM5A_TRUNC | Truncation |
| IRS1_Amp | Amp | KDM5C_TRUNC | Truncation |
| IRS2_Amp | Amp | KDM6A_TRUNC | Truncation |
| JAK2_Amp | Amp | KEAP1_TRUNC | Truncation |
| KDM5A_Amp | Amp | KIT_TRUNC | Truncation |
| KDM6A_Amp | Amp | LATS1_TRUNC | Truncation |
| KDR_Amp | Amp | MAP2K4_TRUNC | Truncation |
| KIT_Amp | Amp | MAP3K1_TRUNC | Truncation |
| KRAS_Amp | Amp | MCL1_TRUNC | Truncation |
| MCL1_Amp | Amp | MED_12_TRUNC | Truncation |
| MDC1_Amp | Amp | MEN1_TRUNC | Truncation |
| MDM2_Amp | Amp | MET_TRUNC | Truncation |
| MDM4_Amp | Amp | NCOR1_TRUNC | Truncation |
| MET_Amp | Amp | NF1_TRUNC | Truncation |
| MITF_Amp | Amp | NF2_TRUNC | Truncation |
| MPL_Amp | Amp | NOTCH1_TRUNC | Truncation |
| MYC_Amp | Amp | NSD1_TRUNC | Truncation |
| MYCL_Amp | Amp | PBRM1_TRUNC | Truncation |
| MYCN_Amp | Amp | PIK3R1_TRUNC | Truncation |
| NBN_Amp | Amp | PTCH1_TRUNC | Truncation |
| NKX2.1_Amp | Amp | PTEN_TRUNC | Truncation |
| NOTCH2_Amp | Amp | PTPRT_TRUNC | Truncation |
| NTRK1_Amp | Amp | RASA1_TRUNC | Truncation |
| PAK1_Amp | Amp | RB1_TRUNC | Truncation |
| PDGFRA_Amp | Amp | RBM10_TRUNC | Truncation |
| PIK3C2G_Amp | Amp | RECQL4_TRUNC | Truncation |
| PIK3CA_Amp | Amp | RNF43_TRUNC | Truncation |
| PIK3R2_Amp | Amp | SETD2_TRUNC | Truncation |
| PMS2_Amp | Amp | SF3B1_TRUNC | Truncation |
| PRKAR1A_Amp | Amp | SMAD4_TRUNC | Truncation |
| PTPRD_Amp | Amp | SMARCA4_TRUNC | Truncation |
| RAC1_Amp | Amp | SMARCB1_TRUNC | Truncation |
| RAD51C_Amp | Amp | SOX9_TRUNC | Truncation |
| RAD52_Amp | Amp | SPEN_TRUNC | Truncation |
| RAFI_Amp | Amp | STAG2_TRUNC | Truncation |
| RARA_Amp | Amp | STK11_TRUNC | Truncation |
| RECQL4_Amp | Amp | TBX3_TRUNC | Truncation |
| RET_Amp | Amp | TET2_TRUNC | Truncation |
| RICTOR_Amp | Amp | TGFBR2_TRUNC | Truncation |
| RIT1_Amp | Amp | TP53_TRUNC | Truncation |
| RNF43_Amp | Amp | TSC1_TRUNC | Truncation |
| RPS6KB2_Amp | Amp | TSC2_TRUNC | Truncation |
| RPTOR_Amp | Amp | VHL_TRUNC | Truncation |
| RUNX1_Amp | Amp | AMER1 | VUS |
| SDHA_Amp | Amp | ABL1 | VUS |
| SDHC_Amp | Amp | AKT1 | VUS |
| SOX17_Amp | Amp | AKT3 | VUS |
| SOX2_Amp | Amp | ALK | VUS |
| SOX9_Amp | Amp | ALOX12B | VUS |
| SPOP_Amp | Amp | APC | VUS |
| SRC_Amp | Amp | AR | VUS |
| TBX3_Amp | Amp | ARAF | VUS |
| TERT_Amp | Amp | ARID1A | VUS |
| TET2_Amp | Amp | ARID1B | VUS |
| TMPRSS2_Amp | Amp | ARID2 | VUS |
| TP63_Amp | Amp | ARID5B | VUS |
| YAP1_Amp | Amp | ASXL1 | VUS |
| Amp_10p | Gain | ASXL2 | VUS |
| Amp_10q | Gain | ATM | VUS |
| Amp_11p | Gain | ATR | VUS |
| Amp_11q | Gain | ATRX | VUS |
| Amp_12p | Gain | AURKA | VUS |
| Amp_12q | Gain | AXIN1 | VUS |
| Amp_13q | Gain | AXIN2 | VUS |
| Amp_14q | Gain | AXL | VUS |
| Amp_15q | Gain | BAP1 | VUS |
| Amp_16p | Gain | BARD1 | VUS |
| Amp_16q | Gain | BBC3 | VUS |
| Amp_17p | Gain | BCOR | VUS |
| Amp_17q | Gain | BLM | VUS |
| Amp_18p | Gain | BMPR1A | VUS |
| Amp_18q | Gain | BRAF | VUS |
| Amp_19p | Gain | BRCA1 | VUS |
| Amp_19q | Gain | BRCA2 | VUS |
| Amp_1p | Gain | BRD4 | VUS |
| Amp_1q | Gain | BTK | VUS |
| Amp_20p | Gain | CARD11 | VUS |
| Amp_20q | Gain | CASP8 | VUS |
| Amp_21q | Gain | CBFB | VUS |
| Amp_22q | Gain | CBL | VUS |
| Amp_2p | Gain | CCND1 | VUS |
| Amp_2q | Gain | CD79B | VUS |
| Amp_3p | Gain | CDH1 | VUS |
| Amp_3q | Gain | CDK12 | VUS |
| Amp_4p | Gain | CDK8 | VUS |
| Amp_4q | Gain | CDKN1A | VUS |
| Amp_5p | Gain | CDKN1B | VUS |
| Amp_5q | Gain | CDKN2A | VUS |
| Amp_6p | Gain | CHEK2 | VUS |
| Amp_6q | Gain | CIC | VUS |
| Amp_7p | Gain | CREBBP | VUS |
| Amp_7q | Gain | CSF1R | VUS |
| Amp_8p | Gain | CTCF | VUS |
| Amp_8q | Gain | CTNNB1 | VUS |
| Amp_9p | Gain | CUL3 | VUS |
| Amp_9q | Gain | DAXX | VUS |
| Amp_Xp | Gain | DDR2 | VUS |
| Amp_Xq | Gain | DICER1 | VUS |
| ARID1A_HomDel | Homdel | DIS3 | VUS |
| ARID5B_HomDel | Homdel | DNMT1 | VUS |
| B2M_HomDel | Homdel | DNMT3A | VUS |
| BAP1_HomDel | Homdel | DNMT3B | VUS |
| BCOR_HomDel | Homdel | DOT1L | VUS |
| BRCA2_HomDel | Homdel | EGFR | VUS |
| CARD11_HomDel | Homdel | EIF1AX | VUS |
| CDKN1B_HomDel | Homdel | EP300 | VUS |
| CDKN2A_HomDel | Homdel | EPHA3 | VUS |
| CDKN2B_HomDel | Homdel | EPHA5 | VUS |
| CRLF2_HomDel | Homdel | EPHB1 | VUS |
| FAT1_HomDel | Homdel | ERBB2 | VUS |
| FLT4_HomDel | Homdel | ERBB3 | VUS |
| FOXL2_HomDel | Homdel | ERBB4 | VUS |
| GATA3_HomDel | Homdel | ERCC2 | VUS |
| JUN_HomDel | Homdel | ERCC4 | VUS |
| NF1_HomDel | Homdel | ERCC5 | VUS |
| PAK1_HomDel | Homdel | ERG | VUS |
| PIK3CD_HomDel | Homdel | ESR1 | VUS |
| PTEN_HomDel | Homdel | ETV1 | VUS |
| PTPRD_HomDel | Homdel | ETV6 | VUS |
| RAD51_HomDel | Homdel | EZH2 | VUS |
| RASA1_HomDel | Homdel | FAM46C | VUS |
| RB1_HomDel | Homdel | FANCA | VUS |
| RET_HomDel | Homdel | FAT1 | VUS |
| SMAD4_HomDel | Homdel | FBXW7 | VUS |
| SUZ12_HomDel | Homdel | FGF4 | VUS |
| TGFBR2_HomDel | Homdel | FGFR1 | VUS |
| TNFRSF14_HomDel | Homdel | FGFR2 | VUS |
| AKT1_hotspot | Hotspot | FGFR3 | VUS |
| ALK_hotspot | Hotspot | FGFR4 | VUS |
| APC_hotspot | Hotspot | FH | VUS |
| AR_hotspot | Hotspot | FLCN | VUS |
| ARID1A_hotspot | Hotspot | FLT1 | VUS |
| BAP1_hotspot | Hotspot | FLT3 | VUS |
| BCOR_hotspot | Hotspot | FLT4 | VUS |
| BRAF_hotspot | Hotspot | FOXA1 | VUS |
| CARD11_hotspot | Hotspot | FOXL2 | VUS |
| CDKN2A_hotspot | Hotspot | FOXP1 | VUS |
| CIC_hotspot | Hotspot | FUBP1 | VUS |
| CTNNB1_hotspot | Hotspot | GATA1 | VUS |
| EGFR_hotspot | Hotspot | GATA2 | VUS |
| EIF1AX_hotspot | Hotspot | GATA3 | VUS |
| EP300_hotspot | Hotspot | GNA11 | VUS |
| ERBB2_hotspot | Hotspot | GNAQ | VUS |
| ERBB3_hotspot | Hotspot | GNAS | VUS |
| ERCC2_hotspot | Hotspot | GRIN2A | VUS |
| ESR1_hotspot | Hotspot | GSK3B | VUS |
| FBXW7_hotspot | Hotspot | HGF | VUS |
| FGFR2_hotspot | Hotspot | HNF1A | VUS |
| FGFR3_hotspot | Hotspot | HRAS | VUS |
| FOXA1_hotspot | Hotspot | IDH1 | VUS |
| GNA11_hotspot | Hotspot | IDH2 | VUS |
| GNAQ_hotspot | Hotspot | IFNGR1 | VUS |
| GNAS_hotspot | Hotspot | IGF1R | VUS |
| HRAS_hotspot | Hotspot | IKBKE | VUS |
| IDH1_hotspot | Hotspot | IKZF1 | VUS |
| IDH2_hotspot | Hotspot | IL7R | VUS |
| KDM6A_hotspot | Hotspot | INPP4A | VUS |
| KIT_hotspot | Hotspot | INPP4B | VUS |
| KRAS_hotspot | Hotspot | INSR | VUS |
| MAP2K1_hotspot | Hotspot | IRF4 | VUS |
| MTOR_hotspot | Hotspot | IRS1 | VUS |
| NFE2L2_hotspot | Hotspot | IRS2 | VUS |
| NOTCH1_hotspot | Hotspot | JAK1 | VUS |
| NRAS_hotspot | Hotspot | JAK2 | VUS |
| PDGFRA_hotspot | Hotspot | JAK3 | VUS |
| PIK3CA_hotspot | Hotspot | KDM5A | VUS |
| PIK3R1_hotspot | Hotspot | KDM5C | VUS |
| PPP2R1A_hotspot | Hotspot | KDM6A | VUS |
| PTEN_hotspot | Hotspot | KDR | VUS |
| PTPN11_hotspot | Hotspot | KEAP1 | VUS |
| RAC1_hotspot | Hotspot | KIT | VUS |
| RB1_hotspot | Hotspot | KLF4 | VUS |
| RET_hotspot | Hotspot | KRAS | VUS |
| RHOA_hotspot | Hotspot | LATS1 | VUS |
| SF3B1_hotspot | Hotspot | LATS2 | VUS |
| SMAD4_hotspot | Hotspot | MAP2K1 | VUS |
| SMARCA4_hotspot | Hotspot | MAP2K4 | VUS |
| SPOP_hotspot | Hotspot | MAP3K1 | VUS |
| STK11_hotspot | Hotspot | MAP3K13 | VUS |
| TP53_hotspot | Hotspot | MAPK1 | VUS |
| TRAF7_hotspot | Hotspot | MAX | VUS |
| VHL_hotspot | Hotspot | MDC1 | VUS |
| AKT1.E17K | Hotspot Allele | MED12 | VUS |
| ALK.F1174L | Hotspot Allele | MEF2B | VUS |
| ALK.F1245V | Hotspot Allele | MEN1 | VUS |
| ALK.R1275Q | Hotspot Allele | MET | VUS |
| APC.R1450. | Hotspot Allele | MITF | VUS |
| APC.R216. | Hotspot Allele | MLH1 | VUS |
| APC.R876. | Hotspot Allele | MPL | VUS |
| BAP1.K25_D34delinsN | Hotspot Allele | MRE11A | VUS |
| BCOR.N1459S | Hotspot Allele | MSH2 | VUS |
| BRAF.V600E | Hotspot Allele | MSH6 | VUS |
| BRAF.V600K | Hotspot Allele | MTOR | VUS |
| CARD11.R337. | Hotspot Allele | MYCN | VUS |
| CDKN2A.H83Y | Hotspot Allele | NBN | VUS |
| CDKN2A.R80. | Hotspot Allele | NCOR1 | VUS |
| CTNNB1.D32Y | Hotspot Allele | NF1 | VUS |
| CTNNB1.S37F | Hotspot Allele | NF2 | VUS |
| CTNNB1.S45F | Hotspot Allele | NFE2L2 | VUS |
| EGFR.E746_A750del | Hotspot Allele | NOTCH1 | VUS |
| EGFR.L858R | Hotspot Allele | NOTCH2 | VUS |
| EGFR.T790M | Hotspot Allele | NOTCH3 | VUS |
| EIF1AX.X113_splice | Hotspot Allele | NOTCH4 | VUS |
| EIF1AX.X6_splice | Hotspot Allele | NRAS | VUS |
| EP300.H1451Q | Hotspot Allele | NSD1 | VUS |
| ERBB2.S310F | Hotspot Allele | NTRK1 | VUS |
| ESR1.D538G | Hotspot Allele | NTRK2 | VUS |
| FBXW7.R479Q | Hotspot Allele | NTRK3 | VUS |
| FGFR3.R248C | Hotspot Allele | PAK1 | VUS |
| FGFR3.S249C | Hotspot Allele | PAK7 | VUS |
| FGFR3 Y373C | Hotspot Allele | PALB2 | VUS |
| GNA11.Q209L | Hotspot Allele | PARK2 | VUS |
| GNAQ.Q209L | Hotspot Allele | PARP1 | VUS |
| GNAQ.Q209P | Hotspot Allele | PAX5 | VUS |
| GNAQ.R183Q | Hotspot Allele | PBRM1 | VUS |
| IDH1.R132C | Hotspot Allele | PDGFRA | VUS |
| IDH1.R132H | Hotspot Allele | PDGFRB | VUS |
| IDH1.R132L | Hotspot Allele | PHOX2B | VUS |
| KIT.A502_Y503dup | Hotspot Allele | PIK3C2G | VUS |
| KIT.L576P | Hotspot Allele | PIK3C3 | VUS |
| KIT.V559D | Hotspot Allele | PIK3CA | VUS |
| KIT.V654A | Hotspot Allele | PIK3CB | VUS |
| KIT.W557_K558del | Hotspot Allele | PIK3CD | VUS |
| KRAS.G12A | Hotspot Allele | PIK3CG | VUS |
| KRAS.G12C | Hotspot Allele | PIK3R1 | VUS |
| KRAS.G12D | Hotspot Allele | PIK3R2 | VUS |
| KRAS.G12R | Hotspot Allele | PLK2 | VUS |
| KRAS.G12V | Hotspot Allele | PMS1 | VUS |
| KRAS.G13D | Hotspot Allele | PMS2 | VUS |
| KRAS.Q61H | Hotspot Allele | POLE | VUS |
| MYCN.P44L | Hotspot Allele | PPP2R1A | VUS |
| NRAS.Q61K | Hotspot Allele | PRDM1 | VUS |
| NRAS.Q61R | Hotspot Allele | PTCH1 | VUS |
| PDGFRA.D842V | Hotspot Allele | PTEN | VUS |
| PIK3CA.E542K | Hotspot Allele | PTPN11 | VUS |
| PIK3CA.E545K | Hotspot Allele | PTPRD | VUS |
| PIK3CA.H1047R | Hotspot Allele | PTPRS | VUS |
| PIK3CA.M1043I | Hotspot Allele | PTPRT | VUS |
| PPP2R1A.P179R | Hotspot Allele | RAC1 | VUS |
| PPP2R1A.S256F | Hotspot Allele | RAD50 | VUS |
| PTEN.R130G | Hotspot Allele | RAD52 | VUS |
| SF3BER625C | Hotspot Allele | RAF1 | VUS |
| SF3BER625H | Hotspot Allele | RARA | VUS |
| SPOP.F133L | Hotspot Allele | RASA1 | VUS |
| TP53.G245S | Hotspot Allele | RB1 | VUS |
| TP53.H179Y | Hotspot Allele | RBM10 | VUS |
| TP53.R158L | Hotspot Allele | RECQL4 | VUS |
| TP53.R175H | Hotspot Allele | REL | VUS |
| TP53.R213. | Hotspot Allele | RET | VUS |
| TP53.R248Q | Hotspot Allele | RHOA | VUS |
| TP53.R248W | Hotspot Allele | RICTOR | VUS |
| TP53.R273C | Hotspot Allele | RNF43 | VUS |
| TP53.R273H | Hotspot Allele | ROS1 | VUS |
| TP53.R282W | Hotspot Allele | RPS6KA4 | VUS |
| TP53.R342. | Hotspot Allele | RPS6KB2 | VUS |
| TP53.V157F | Hotspot Allele | RPTOR | VUS |
| TP53.X225_splice | Hotspot Allele | RUNX1 | VUS |
| TP53.Y220C | Hotspot Allele | RYBP | VUS |
| TP53.Y234C | Hotspot Allele | SDHA | VUS |
| TRAF7.N520S | Hotspot Allele | SETD2 | VUS |
| U2AF1.S34F | Hotspot Allele | SF3B1 | VUS |
| VHL.X114_splice | Hotspot Allele | SMAD2 | VUS |
| Del_10p | Loss | SMAD3 | VUS |
| Del_10q | Loss | SMAD4 | VUS |
| Del_11p | Loss | SMARCA4 | VUS |
| Del_11q | Loss | SMARCB1 | VUS |
| Del_12p | Loss | SMARCD1 | VUS |
| Del_12q | Loss | SMO | VUS |
| Del_13q | Loss | SOX_17 | VUS |
| Del_14q | Loss | SOX2 | VUS |
| Del_15q | Loss | SOX9 | VUS |
| Del_16p | Loss | SPEN | VUS |
| Del_16q | Loss | SPOP | VUS |
| Del_17p | Loss | STAG2 | VUS |
| Del_17q | Loss | STK11 | VUS |
| Del_18p | Loss | SUFU | VUS |
| Del_18q | Loss | SYK | VUS |
| Del_19p | Loss | TBX3 | VUS |
| Del_19q | Loss | TERT | VUS |
| Del_1p | Loss | TET1 | VUS |
| Del_1q | Loss | TET2 | VUS |
| Del_20p | Loss | TGFBR1 | VUS |
| Del_20q | Loss | TGFBR2 | VUS |
| Del_21q | Loss | TMPRSS2 | VUS |
| Del_22q | Loss | TNFAIP3 | VUS |
| Del_2p | Loss | TOP1 | VUS |
| Del_2q | Loss | TP53 | VUS |
| Del_3p | Loss | TP63 | VUS |
| Del_3q | Loss | TRAF7 | VUS |
| Del_4p | Loss | TSC1 | VUS |
| Del_4q | Loss | TSC2 | VUS |
| Del_5p | Loss | TSHR | VUS |
| Del_5q | Loss | U2AF1 | VUS |
| Del_6p | Loss | VHL | VUS |
| Del_6q | Loss | XPO1 | VUS |
| Del_7p | Loss | | |

Clinical information. The sex of the patient is included as a binary feature. While the age at screening has been linked to the incidence of some cancer types, it was excluded from the feature set due to the ambiguity that arises for patients with multiple independent cancer classification or those earlier ages of classification associated with germline pathogenic alterations.

### Classification

A multi-class classifier was built using the random forest algorithm. The random forest ensemble learning method may be suited for this complex classification problem due to its ability to better accommodate large numbers of potentially informative features, arbitrary combinations of features, and the imbalanced class representation of the cohort (i.e., wide range in the prevalence of individual cancer types) as compared to alternative approaches. Moreover, random forest classifiers quantify the relative importance of each variable, enabling the classifier to provide valuable context for clinical interpretations. The imbalanced representation was resolved by equal stratified sampling of tumor types during learning. Specifically, the portion of data used to build each tree included an equal number of samples drawn from each cancer type equal to 80% of the size of the smallest class. This sampling exacerbates the tendency of ensemble classification algorithms, including random forests, to return ambivalent confidence scores even in cases of high certainty. For the primary performance metric, Cohen's kappa, which takes into account the degree of agreement expected by chance between the output and the reference labels, may be used.

### Calibration

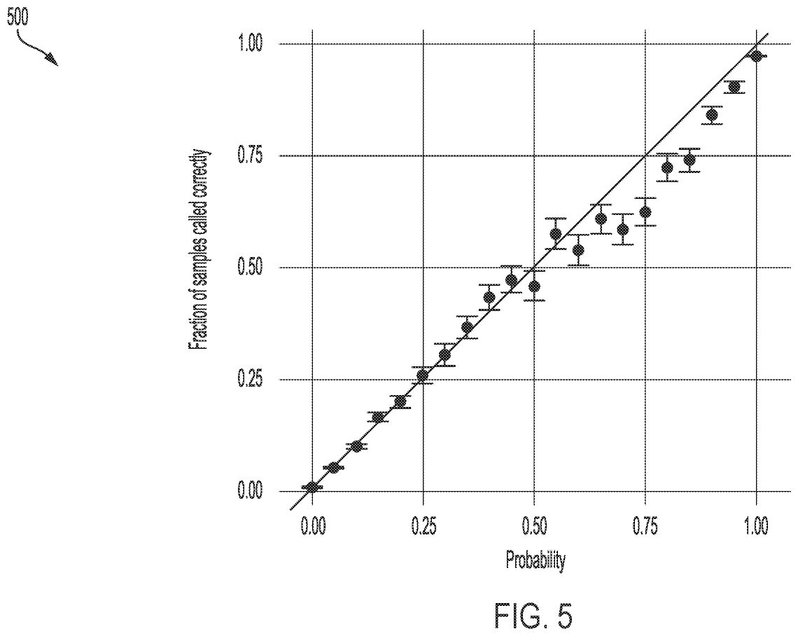

The raw classifier scores may be adjusted to match the classification probability using Platt scaling, a multinomial regression. Classification scores from ensemble machine learning methods such as random forest trees often do not approach the extremes of 0 or 1, resulting in a sigmoid shaped distribution relative to the probability. This mismatch between classifier score and probability tends to be exacerbated by stratified sampling of classes. The results of the random forest classifier were calibrated to approximate the empirical accuracy of predictions, using multinomial logistic regression with an elastic-net penalty using the glmnet package in R. Naive calibration tends to lead to a large loss of sensitivity for less common and less distinctive tumor types, especially those that share features with a common tumor type. This effect may be mitigated with slight down-sampling of more common tumor types to maximize the mean balanced accuracy across cancer types. Twenty repeats of five-fold cross-validation were used to determine the robustness of classifier predictions. The agreement between calibrated probability and prediction accuracy is shown in FIG. 5.

### Circulating DNA

The classifier was applied to predict cancer type for two separate groups of patients with circulating tumor DNA (cfDNA) sequencing data. First, 19 patients with prostate, bladder, and testicular cancer were selected from a larger cohort with MSK-IMPACT sequencing of cfDNA based on the detection of mutations with a median variant allele fraction greater than 0.10. None of these patients were included in the classifier training set. Second, cancer types using ctDNA whole exome sequencing results was predicted.

An example data structure of a potential training dataset to train a classifier according to certain embodiments may include, for example, fields such as CANCER_TYPE, CANCER_TYPE_DETAILED, SAMPLE_TYPE, PRIMARY_SITE, METASTATIC_SITE, Cancer_Type, Classification_Category, Gender_F, LogSNV_Mb, and LogINDEL_Mb. Example values corresponding to the fields may comprise, for example: AKT1, AKT2, AKT3, ALK, ALOX12B, AMER1, APC, AR, ARAF, and ARID1A.

An example data structure of a potential patient sample dataset that may be input to a model to obtain a prediction may, according to certain embodiments, be represented by the following (in JavaScript Object Notation (JSON) format):

### B. Computing and Network Environment Text

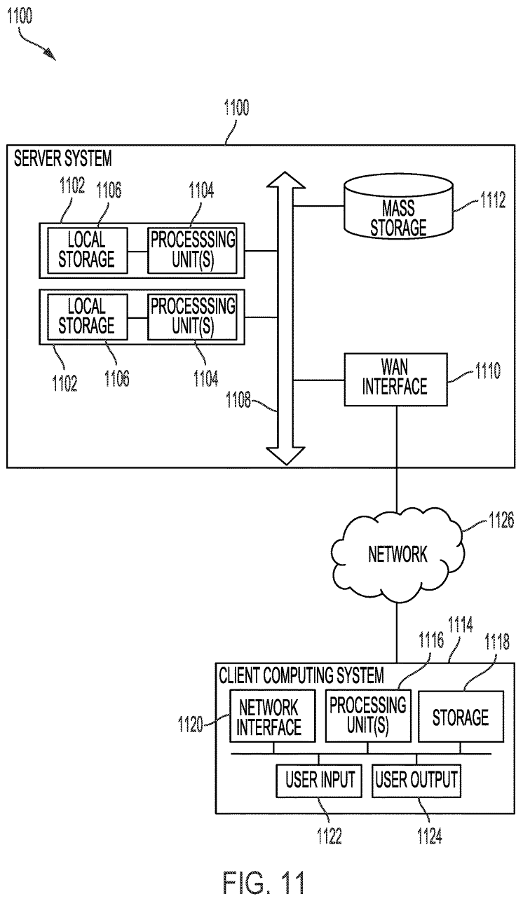

Various operations described herein can be implemented on computer systems, which can be of generally design. FIG. 11 shows a simplified block diagram of a representative server system 1100, client computer system 1114, and network 1126 usable to implement certain embodiments of the present disclosure. In various embodiments, server system 1100 or similar systems can implement services or servers described herein or portions thereof. Client computer system 1114 or similar systems can implement clients described herein.

Server system 1100 can have a modular design that incorporates a number of modules 1102 (e.g., blades in a blade server embodiment); while two modules 1102 are shown, any number can be provided. Each module 1102 can include processing unit(s) 1104 and local storage 1106.

Processing unit(s) 1104 can include a single processor, which can have one or more cores, or multiple processors. In some embodiments, processing unit(s) 1104 can include a general-purpose primary processor as well as one or more special-purpose co-processors such as graphics processors, digital signal processors, or the like. In some embodiments, some or all processing units 1104 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1104 can execute instructions stored in local storage 1106. Any type of processors in any combination can be included in processing unit(s) 1104.

Local storage 1106 can include volatile storage media (e.g., DRAM, SRAM, SDRAM, or the like) and/or non-volatile storage media (e.g., magnetic or optical disk, flash memory, or the like). Storage media incorporated in local storage 1106 can be fixed, removable or upgradeable as desired. Local storage 1106 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1104 need at runtime. The ROM can store static data and instructions that are needed by processing unit(s) 1104. The permanent storage device can be a non-volatile read-and-write memory device that can store instructions and data even when module 1102 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1106 can store one or more software programs to be executed by processing unit(s) 1104, such as an operating system and/or programs implementing various server functions such as functions of the system 100 (e.g., the classification system 102 and the sequencer 104) in FIG. 1D, or any other system described herein.

“Software” refers generally to sequences of instructions that, when executed by processing unit(s) 1104 cause server system 1100 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1104. Software can be implemented as a single program or a collection of separate programs or program modules that interact as desired. From local storage 1106 (or non-local storage described below), processing unit(s) 1104 can retrieve program instructions to execute and data to process in order to execute various operations described above.

In some server systems 1100, multiple modules 1102 can be interconnected via a bus or other interconnect 1108, forming a local area network that supports communication between modules 1102 and other components of server system 1100. Interconnect 1108 can be implemented using various technologies including server racks, hubs, routers, etc.

A wide area network (WAN) interface 1110 can provide data communication capability between the local area network (interconnect 1108) and the network 1126, such as the Internet. Technologies can be used, including wired (e.g., Ethernet, IEEE 802.3 standards) and/or wireless technologies (e.g., Wi-Fi, IEEE 802.11 standards).

In some embodiments, local storage 1106 is intended to provide working memory for processing unit(s) 1104, providing fast access to programs and/or data to be processed while reducing traffic on interconnect 1108. Storage for larger quantities of data can be provided on the local area network by one or more mass storage subsystems 1112 that can be connected to interconnect 1108. Mass storage subsystem 1112 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1112. In some embodiments, additional data storage resources may be accessible via WAN interface 1110 (potentially with increased latency).

Server system 1100 can operate in response to requests received via WAN interface 1110. For example, one of modules 1102 can implement a supervisory function and assign discrete tasks to other modules 1102 in response to received requests. Work allocation techniques can be used. As requests are processed, results can be returned to the requester via WAN interface 1110. Such operation can generally be automated. Further, in some embodiments, WAN interface 1110 can connect multiple server systems 1100 to each other, providing scalable systems capable of managing high volumes of activity. Techniques for managing server systems and server farms (collections of server systems that cooperate) can be used, including dynamic resource allocation and reallocation.

Server system 1100 can interact with various user-owned or user-operated devices via a wide-area network such as the Internet. An example of a user-operated device is shown in FIG. 11 as client computing system 1114. Client computing system 1114 can be implemented, for example, as a consumer device such as a smartphone, other mobile phone, tablet computer, wearable computing device (e.g., smart watch, eyeglasses), desktop computer, laptop computer, and so on.

For example, client computing system 1114 can communicate via WAN interface 1110. Client computing system 1114 can include computer components such as processing unit(s) 1116, storage device 1118, network interface 1120, user input device 1122, and user output device 1124. Client computing system 1114 can be a computing device implemented in a variety of form factors, such as a desktop computer, laptop computer, tablet computer, smartphone, other mobile computing device, wearable computing device, or the like.

Processor 1116 and storage device 1118 can be similar to processing unit(s) 1104 and local storage 1106 described above. Suitable devices can be selected based on the demands to be placed on client computing system 1114; for example, client computing system 1114 can be implemented as a “thin” client with limited processing capability or as a high-powered computing device. Client computing system 1114 can be provisioned with program code executable by processing unit(s) 1116 to enable various interactions with server system 1100 of a message management service such as accessing messages, performing actions on messages, and other interactions described above. Some client computing systems 1114 can also interact with a messaging service independently of the message management service.

Network interface 1120 can provide a connection to the network 1126, such as a wide area network (e.g., the Internet) to which WAN interface 1110 of server system 1100 is also connected. In various embodiments, network interface 1120 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 1122 can include any device (or devices) via which a user can provide signals to client computing system 1114; client computing system 1114 can interpret the signals as indicative of particular user requests or information. In various embodiments, user input device 1122 can include any or all of a keyboard, touch pad, touch screen, mouse or other pointing device, scroll wheel, click wheel, dial, button, switch, keypad, microphone, and so on.

User output device 1124 can include any device via which client computing system 1114 can provide information to a user. For example, user output device 1124 can include a display to display images generated by or delivered to client computing system 1114. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1124 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1104 and 1116 can provide various functionality for server system 1100 and client computing system 1114, including any of the functionality described herein as being performed by a server or client, or other functionality associated with message management services.

It will be appreciated that server system 1100 and client computing system 1114 are illustrative and that variations and modifications are possible. Computer systems used in connection with embodiments of the present disclosure can have other capabilities not specifically described here. Further, while server system 1100 and client computing system 1114 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

Various potential embodiments of the disclosure include:

Embodiment A: A method for classifying tumor origin sites, the method comprising: sequencing genetic material in a tissue sample from a subject to generate a subject sample dataset comprising one or more subject genes and one or more subject gene alteration categories; applying a predictive model to the subject sample dataset to generate one or more cancer origin site classifications, the predictive model having been trained using a training dataset generated from sequence reads corresponding to genetic material from a cohort of study subjects with known cancers, the training dataset comprising one or more genes, one or more gene alteration categories corresponding to the one or more genes, and one or more labels characterizing tumor origin sites for the known cancers of the study subjects in the cohort; and storing, in one or more data structures, an association between the subject and the one or more cancer origin site classifications

Embodiment B: The method of Embodiment A, wherein the predictive model is a random forest classification model.

Embodiment C: The method of either Embodiment A or B, wherein a feature set for the predictive model comprises one or more categories selected from a group consisting of mutations, indels, focal amplifications and deletions, broad copy number gains and losses, structural rearrangements, mutation signatures, mutation rate, and sex.

Embodiment D: The method of any of Embodiments A-C, wherein classifier scores for the predictive model were calibrated using multinomial logistic regression to match empirically observed classification probabilities.

Embodiment E: The method of any of Embodiments A-D, further comprising training the predictive model.

Embodiment F: The method of any of Embodiments A-E, wherein the predictive model is trained using supervised learning.

Embodiment G: The method of any of Embodiments A-F, wherein the predictive model is trained using unsupervised learning.

Embodiment H: The method of any of Embodiments A-G, further comprising generating the training dataset.

Embodiment I: The method of any of Embodiments A-H, wherein generating the training dataset comprises acquiring, from a sequencing device, the sequence reads corresponding to the genetic material from the study subjects in the cohort, and using the sequence reads to generate the training dataset.

Embodiment J: The method of any of Embodiments A-I, wherein the cohort excludes study subjects with rare cancers not in the top 30 most common cancer types.

Embodiment K: The method of any of Embodiments A-J, wherein the training dataset comprises gene alteration categories comprising one or more selected from a group consisting of gene amplification (AMP), chromosome gain, homozygous deletion, hotspot, allele, chromosome loss, promoter, signature, structural variant (SV), truncation, and variant of unknown significance (VUS).

Embodiment L: The method of any of Embodiments A-K, wherein the one or more labels indicate whether a set of genes in the training dataset is from a cancer subject in the cohort of study subjects.

Embodiment M: The method of any of Embodiments A-L, wherein the predictive model is configured to accept data on genes and gene alterations as inputs and to provide one or more cancer origin site classifications as output.

Embodiment N: The method of any of Embodiments A-M, wherein the one or more cancer origin site classifications identify at least one of an internal organ of the subject or a cancer type.

Embodiment O: The method of any of Embodiments A-N, wherein the predictive model is further configured to generate a confidence score for each cancer origin site classification.

Embodiment P: The method of any of Embodiments A-O, wherein each confidence score corresponds with a likelihood of a cancer origin site for a tumor.

Embodiment Q: A system for classifying tumor origin sites, the system comprising a computing device having one or more processors configured to: acquire, from a sequencing device, sequence reads corresponding to genetic material in a tissue sample from a subject; generate, using the sequence reads, a subject sample dataset comprising one or more subject genes and one or more subject gene alteration categories; and apply a predictive model to the subject sample dataset to generate one or more cancer origin site classifications, the predictive model having been trained using a training dataset generated using sequence reads corresponding to genetic material from a cohort of study subjects with known cancers, the training dataset comprising one or more genes, one or more gene alteration categories corresponding to the one or more genes, and one or more labels characterizing tumor origin sites for the known cancers of the study subjects in the cohort.

Embodiment R: The system of Embodiment Q, wherein the one or more processors are further configured to store, in one or more data structures, an association between the subject and the one or more cancer origin site classifications.

Embodiment S: The system of either Embodiment Q or R, wherein the predictive model is a random forest classification model.

Embodiment T: The system of any of Embodiments Q-S, wherein the one or more processors are further configured to train the predictive model such that it is configured to accept data on genes and gene alterations as inputs and to provide one or more cancer origin site classifications as output.

Embodiment U: The system of any of Embodiments Q-T, wherein the one or more processors are configured to generate the training dataset using the sequence reads corresponding to the genetic material from the study subjects in the cohort.

Embodiment V: The system of any of Embodiments Q-U, wherein the predictive model trained such that it is configured to accept data on genes and gene alterations as inputs and to provide one or more cancer origin site classifications as output.

Embodiment W: The system of any of Embodiments Q-V, wherein the predictive model is further configured to generate a confidence score for each cancer origin site classification.

Embodiment X: The system of any of Embodiments Q-W, wherein each confidence score corresponds with a likelihood of a cancer origin site for a tumor.

Embodiment Y: A system for determining sites of origin for cancer based on sequencing of genes, the system comprising one or more processors configured to: obtain a training dataset comprising a plurality of sample-derived genetic sequences corresponding to a plurality of cancer subjects, each sample defining a set of genes and a category, the category of each sample defining at least one alteration to the set of genes and/or at least one genomic alteration in the sample; train, using the plurality of sample genetic sequences, a classification model configured to generate likelihoods for corresponding cancer origin sites; acquire, via a sequencer, a genetic sequence corresponding to a subject, the genetic sequence including a set of genes and a category, the category of the genetic sequence defining a nature of alteration to the set of genes in the genetic sequence; and apply the classification model to the genetic sequence to determine a set of likelihoods for a corresponding set of origin sites of cancers, each likelihood indicating a probability measure that the genetic sequence correlates with a presence of cancer at a corresponding origin site.

Embodiment Z: The system of Embodiment Y, wherein the classification model is trained as a random forest classification model.

Embodiment AA: The system of either Embodiment Y or Z, wherein the one more processors are configured to generate the training dataset using sequence reads from the sequencer.

While the disclosure has been described with respect to specific embodiments, one skilled in the art will recognize that numerous modifications are possible. Embodiments of the disclosure can be realized using a variety of computer systems and communication technologies including but not limited to specific examples described herein.

Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method for classifying tumor origin sites, the method comprising:
sequencing genetic material in a tissue sample from a subject to generate a subject sample dataset comprising one or more subject genes and one or more subject gene alteration categories;
applying a predictive model to the subject sample dataset to generate one or more cancer origin site classifications, the predictive model having been trained using a training dataset generated from sequence reads corresponding to genetic material from a cohort of study subjects with known cancers, the training dataset comprising one or more genes, one or more gene alteration categories corresponding to the one or more genes, and one or more labels characterizing tumor origin sites for the known cancers of the study subjects in the cohort; and
storing, in one or more data structures, an association between the subject and the one or more cancer origin site classifications.

2. The method of claim 1, wherein the predictive model is a random forest classification model.

3. The method of claim 2, wherein a feature set for the predictive model comprises one or more categories selected from a group consisting of mutations, indels, focal amplifications and deletions, broad copy number gains and losses, structural rearrangements, mutation signatures, mutation rate, and sex.

4. The method of claim 3, wherein classifier scores for the predictive model were calibrated using multinomial logistic regression to match empirically observed classification probabilities.

5. The method of claim 1, further comprising training the predictive model using supervised or unsupervised learning.

6. The method of claim 1, further comprising generating the training dataset.

7. The method of claim 6, wherein generating the training dataset further comprises acquiring, from a sequencing device, the sequence reads corresponding to the genetic material from the cohort of study subjects, and using the sequence reads to generate the training dataset.

8. The method of claim 1, wherein the cohort excludes study subjects with rare cancers not in the top 30 most common cancer types.

9. The method of claim 1, wherein the training dataset comprises gene alteration categories comprising one or more selected from a group consisting of gene amplification (AMP), chromosome gain, homozygous deletion, hotspot, allele, chromosome loss, promoter, signature, structural variant (SV), truncation, and variant of unknown significance (VUS).

10. The method of claim 1, wherein the one or more labels indicate whether a set of genes in the training dataset is from a cancer subject in the cohort of study subjects.

11. The method of claim 1, wherein the predictive model is configured to accept data on genes and gene alterations as inputs and to provide one or more cancer origin site classifications as output.

12. The method of claim 11, wherein the one or more cancer origin site classifications identify at least one of an internal organ of the subject or a cancer type.

13. The method of claim 11, wherein the predictive model is further configured to generate a confidence score for each cancer origin site classification.

14. The method of claim 13, wherein each confidence score corresponds with a likelihood of a cancer origin site for a tumor.

15. A system for classifying tumor origin sites, the system comprising a computing device having one or more processors configured to:
acquire, from a sequencing device, sequence reads corresponding to genetic material in a tissue sample from a subject;
generate, using the sequence reads, a subject sample dataset comprising one or more subject genes and one or more subject gene alteration categories;
apply a predictive model to the subject sample dataset to generate one or more cancer origin site classifications, the predictive model having been trained using a training dataset generated using sequence reads corresponding to genetic material from a cohort of study subjects with known cancers, the training dataset comprising one or more genes, one or more gene alteration categories corresponding to the one or more genes, and one or more labels characterizing tumor origin sites for the known cancers of the study subjects in the cohort; and
store, in one or more data structures, an association between the subject and the one or more cancer origin site classifications.

16. The system of claim 15, wherein the predictive model is a random forest classification model.

17. The system of claim 15, wherein the one or more processors are further configured to train the predictive model such that it is configured to accept data on genes and gene alterations as inputs and to provide one or more cancer origin site classifications as output.

18. The system of claim 15, wherein the one or more processors are further configured to generate the training dataset using the sequence reads corresponding to the genetic material from the study subjects in the cohort.

19. The system of claim 15, wherein the predictive model is further configured to generate a confidence score for each cancer origin site classification, wherein each confidence score corresponds to a likelihood of a cancer origin site for a tumor.

20. A system for determining sites of origin for cancer based on sequencing of genes, the system comprising one or more processors configured to:
obtain a training dataset comprising a plurality of sample-derived genetic sequences corresponding to a plurality of cancer subjects, each sample defining a set of genes and a category, the category of each sample defining at least one alteration to the set of genes and/or at least one genomic alteration in the sample;
train, using the plurality of sample genetic sequences, a classification model configured to generate likelihoods for corresponding cancer origin sites;
acquire, via a sequencer, a genetic sequence corresponding to a subject, the genetic sequence including a set of genes and a category, the category of the genetic sequence defining a nature of alteration to the set of genes in the genetic sequence; and
apply the classification model to the genetic sequence to determine a set of likelihoods for a corresponding set of origin sites of cancers, each likelihood indicating a probability measure that the genetic sequence correlates with a presence of cancer at a corresponding origin site.
